(19) United States
(12) Patent Application Publication
(10) Pub. No.: US 2024/0174684 A1
(43) Pub. Date: May 30, 2024

(54) INHIBITORS OF cGAS ACTIVITY AS THERAPEUTIC AGENTS

(71) Applicant: BELLBROOK LABS, LLC, Madison, WI (US)

(72) Inventors: Robert G. Lowery, Madison, WI (US); Meera Kumar, Madison, WI (US); Matthew Boxer, Frederick, MD (US); David Maloney, Frederick, MD (US)

(21) Appl. No.: 18/275,190

(22) PCT Filed: Feb. 11, 2022
(86) PCT No.: PCT/US2022/016073

§ 371 Date: Jul. 31, 2023

Related U.S. Application Data
(60) Provisional application No. 63/148,201, filed on Feb. 11, 2021.

(51) Int. Cl. C07D 491/048; A61K 31/519; A61K 31/5377; A61P 37/06

(52) U.S. Cl. CPC C07D 491/048; A61K 31/519; A61K 31/5377; A61P 37/06

(57) ABSTRACT

This disclosure relates to compounds, pharmaceutical compositions comprising them, and methods of using the compounds and compositions for treating or preventing inappropriate activation of a type I interferon (IFN) response in a subject in need thereof.

### CROSS-REFERENCE TO RELATED APPLICATIONS

This application claims the benefit of priority of U.S. Provisional Patent Application No. 63/148,201, filed Feb. 11, 2021, which is incorporated herein by reference in its entirety.

### BACKGROUND OF THE INVENTION

### Field of the Invention

This disclosure relates to compounds, pharmaceutical compositions comprising them, and methods of using the compounds and compositions for treating or preventing inappropriate activation of a type I interferon (IFN) response in a subject in need thereof.

### Description of Related Art

Lupus is the second most prevalent autoimmune disease and affects at least 300,000 people in the U.S. and millions worIckide; it causes severe pain and suffering, which are exacerbated by exposure to sunlight, inability to work and premature death for millions of people worldwide, and there are no curative therapies. Most investigational lupus drugs target the downstream effects of type I IFNs. They include mAbs that block IFNα or IFNAR1, blocking IFNAR1 signal transduction; e.g., JAK inhibitors, targeting cell types activated by type I IFNs; e.g., B- and T-cells.

Cyclic GMP-AMP synthase (cGAS) (UniProtKB-Q8N884) is a recently discovered enzyme that acts as a DNA sensor to elicit an immune response to pathogens via activation of the stimulator of interferon genes (STING) receptor. Shortly after its discovery in 2013, aberrant activation of cGAS by self-DNA was shown to underlie debilitating and sometimes fatal autoimmune diseases, such as systemic lupus erythematosus (SLE), scleroderma, and Aicardi-Goutieres Syndrome (AGS). Knockout studies in animal models have indicated that inhibiting cGAS is a promising approach for therapeutic intervention. Additionally, recent studies have shown that the cGAS-STING pathway plays a key role in the innate immune response to tumors, and stimulation of the pathway is a promising strategy being tested clinically for cancer immunotherapy. However, with the exception of a compound related to the antimalarial hydroxychloroquine, which inhibits cGAS by binding to DNA, there are no reports of a molecule that has been tested in an animal model for lupus or any other autoimmune disease.

No drugs have been approved specifically for AGS or any other monogenic type I interferonopathies. Current treatment options are limited to intravenous or oral immunosuppressors and intravenous immunoglobulins during the acute phases, with often only partial control of the flares. Similarly, SLE is treated with over-the counter anti-inflammatories, corticosteroids, and immunosupressives, such as cyclophosphamide and methotrexate, with serious side effects including cancer. The only targeted therapy approved for SLE is BENLYSTA (belimumab), a monoclonal antibody (mAb) against B-cell activating factor (BAFF). BENLYSTA reduces the risk of severe flares and allows lower doses of immunosuppressive in most patients but is not curative.

Accordingly, there remains a need for compounds that can effectively inhibit cGAS activity and treat diseases resulting from aberrant activation of cGAS.

### SUMMARY OF THE INVENTION

It is against the above background that the present invention provides certain advantages over the prior art.

Although this invention as disclosed herein is not limited to specific advantages or functionalities (such for example, novel inhibitors of cGAS activity), the invention provides a compound of formula (I):

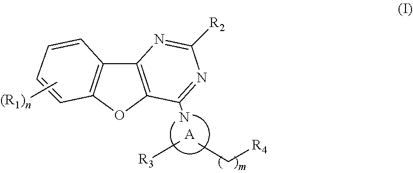

or a pharmaceutically acceptable salt, N-oxide, and/or solvate or hydrate thereof, wherein:
m is an integer of 1, 2, or 3;
n is an integer of 0, 1, 2, 3, or 4:
ring A represents a 4 to 8 membered heterocyclyl ring;
each R¹ is independently selected from halogen, —NO₂, —CN, C₁-C₆ alkyl, C₁-C₆ haloalkyl, —OH, C₁-C₆ alkoxy, and C₁-C₆ haloalkoxy;
R² is hydrogen, C alkyl, or C haloalkyl;
R³ is —CO₂R₅, —COR₅, —C(O)NR⁵ R₆, —CONH—OH, —S(O)₀₋₂—R₅, —SO₂OR₅, or —SO₂NR⁵R₆; and
R⁴ is —C(O)NR⁶ R₇, —CO₂R₇, —SO₂OR₇, or —SO₂NR⁶R₇,
wherein
R⁵ is hydrogen or C₁-C₆ alkyl;
R⁶ is hydrogen, C₁-C₆ alkyl, or C₁-C₆ haloalkyl;
R⁷ is selected from the group consisting of aryl(C₀-C₄ alkyl) optionally substituted with one or more R₉, heteroaryl(C₀-C₄ alkyl) optionally substituted with one or more R₉, heterocyclyl(C₀-C₄ alkyl) optionally substituted with one or more R₈, and cycloalkyl(C₀-C₄ alkyl) optionally substituted with one or more R₈;
each R₈ is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R₆, —C(O)OR₆, and —C(O)NR; R₆, or two R₆ form an oxo;
each R₉ is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy. C₁-C₆ haloalkoxy, aryl(C₀-C₁ alkyl) optionally substituted with one or more R₁₀, heteroaryl(C₀-C₁ alkyl) optionally substituted with one or more R₁₀, heterocyclyl(C₀-C₁ alkyl) optionally substituted with one or more R₁₀, and cycloalkyl(C₀-C₁ alkyl) optionally substituted with one or more R₁₀, and
each R₁₀ is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, and -methyl—OH.

In certain embodiments, the compound of formula (I) is not: (2S,4R)-1-(2-methylbenzofuro[3,2-d]pyrimidin-4-yl)-4-(2-oxo-2-(pyridin-4-ylamino)ethyl)pyrrolidine-2-carboxylic acid. (2S,4R)-4-(2-((1H-pyrazol-4-yl)amino)-2-oxoethyl)-1-(2-methylbenzofuro[3,2-d]pyrimidin-4-yl)pyrrolidine-2-carboxylic acid, (2S,4R)-1-(2-methylbenzofuro[3,2-d]pyrimidin-4-yl)-4-(2-oxo-2-(pyridin-2-ylamino)ethyl)pyrrolidine-2-carboxylic acid, (2S,4R)-4-(2-(cyclopentyl-amino)-2-oxoethyl)-1-(2-methylbenzofuro[3,2-d]pyrimidin-4-yl)pyrrolidine-2-carboxylic acid; or (2S,4R)-1-(2-methylbenzofuro[3,2-d]pyrimidin-4-yl)-4-(2-oxo-2-(phenylamino)ethyl)pyrrolidine-2-carboxylic acid.

Another aspect of the disclosure provides pharmaceutical compositions comprising one or more of compounds of the disclosure (e.g., compounds as described above with respect to formula (I)) and an appropriate carrier, solvent, adjuvant, or diluent.

The disclosure also provides a method for treating or preventing inappropriate activation of a type I interferon (IFN) response in a subject in need thereof, comprising administering to the subject an effective amount of one or more of the compounds of formula (I), as discussed above.

In embodiments of the methods disclosed herein, the inappropriate activation of a type I IFN response comprises an autoimmune disorder (e.g., Aicardi-Goutieres Syndrome (AGS), retinal vasculopathy with cerebral leukodystropy (RVCL), lupus erythematosus (SLE), scleroderma, or Sjögren's syndrome (SS)). Other aspects of the disclosure will be apparent to the person of ordinary skill in the art in view of the disclosure herein.

Another aspect of the disclosure provides a method of treating an autoimmune disorder, the method comprising administering to a subject in need of such treatment an effective amount of one or more compounds of the disclosure (e.g., compounds as described above with respect to formula (1)) or pharmaceutical compositions of the disclosure.

In certain embodiments of this aspect, the autoimmune disorder is AGS, RVCL, SLE, scleroderma, SS, age-related macular degeneration (AMD), pancreatitis, ischemia (e.g., ischemic injury), inflammatory bowel disease (IBD), nonalcoholic steatohepatitis (NASH), or Parkinson's disease.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure and, together with the description, serve to explain the principles and operation of the disclosure.

FIG. 1 is a schematic showing that activation of cGAS by cytoplasmic DNA initiates activation of the innate immune response via induction of Type I interferons (IFN-1).

FIG. 2 is a bar graph of the effect of Compounds 4 and 5 at 20 μM on IFNß mRNA levels in human THP-1 Dual cells, normalized to β-actin. BX is the BTK1 inhibitor BX-795 (N-[3-[[5-iodo-4-[[3-[(2-thienylcarbonyl)amino]propyl]amino]-2-pyrimidinyl]amino]phenyl]-1-pyrrolidinecarboxamide) at 1 μM.

FIG. 3 shows dose dependent stabilization of cGAS by Compound 5 in cells measured using cellular thermal shift assay (CETSA). Panel A is an image of a western blot of the stabilization measure; panel B is a bar graph of the stabilization measure. Heat treatment was conducted at 51.5 ° C.

FIG. 4 shows plasma (a) and plasma log (b) concentration-time profile of compound 5 (BBL-100455) over time for C57BL/6 mice following intravenous (IV) and oral (PO) administration (n=3).

### DETAILED DESCRIPTION OF THE INVENTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed materials and methods provide improvements in treatment of diseases or disorders associated with aberrant activation of cGAS. Specifically, the inventors found that the compounds of the disclosure inhibit cGAS activity, and thus can treat or prevent inappropriate activation of a type I IFN response. The compounds of the disclosure are defined generically as with respect to formula (I), and to various subgenera as defined herein below.

Accordingly, one aspect of the disclosure provides compounds of formula (I) as described above:

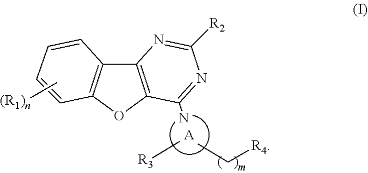

In certain embodiments, the compound of formula (I) is not: (2S,4R)-1-(2-methylbenzofuro[3,2-d]pyrimidin-4-yl)-4-(2-oxo-2-(pyridin-4-ylamino)ethyl)-pyrrolidine-2-carboxylic acid. (2S,4R)-4-(2-((1H-pyrazol-4-yl)amino)-2-oxoethyl)-1-(2-methyl-benzofuro[3,2-d]pyrimidin-4-yl)pyrrolidine-2-carboxylic acid, (28.4R)-1-(2-methylbenzofuro[3.2-d]pyrimidin-4-yl)-4-(2-oxo-2-(pyridin-2-ylamino)ethyl)pyrrolidine-2-carboxylic acid, (2S,4R)-4-(2-(cyclopentylamino)-2-oxoethyl)-1-(2-methylbenzofuro[3,2-d]pyrimidin-4-yl)pyrrolidine-2-carboxylic acid; or (2S,4R)-1-(2-methylbenzofuro[3,2-d]pyrimidin-4-yl)-4-(2-oxo-2-(phenylamino)-ethyl)pyrrolidine-2-carboxylic acid.

One embodiment of the disclosure provides compounds of formula (I) as described herein, wherein n is 0, 1, 2, or 3. In certain embodiments, compounds of formula (I) are wherein n is 0. 1. or 2. In certain embodiments, compounds of formula (I) are wherein n is 0 or 1. In certain embodiments of the compounds of formula (I) as described herein n is 2, 3, or 4, and each R; is the same. In certain embodiments of the compounds of formula (I) as described herein n is 2, 3, or 4, and each R₁ is different. One embodiment of the disclosure provides compounds of formula (I) as described herein, wherein n is 0.

One embodiment of the disclosure provides compounds of formula (1) as described herein, wherein R₁ is halogen, —NO₂, —CN, C₁-C₄ alkyl, C₁-C₄ haloalkyl, —OH, C₁-C₄ alkoxy, or C₁-C₄ haloalkoxy. In certain embodiments, each R₁ is independently selected from halogen, C₁-C₆ alkyl. C₁-C₆ haloalkyl, —OH, and C₁-C₆ alkoxy. In certain embodiments of the compounds of formula (I) each R₁ is independently selected from C₁-C₃ alkyl, —OH, and C₁-C₃ alkoxy.

Another embodiment of the disclosure provides compounds of formula (I) as described herein, wherein R₂ is a hydrogen or a C₁-C₆ alkyl. In certain embodiments, R₂ is hydrogen or C₁-C₄ alkyl. In certain embodiments. R₂ is hydrogen. In certain embodiments, R₂ is C₁-C₄ alkyl, such as methyl.

In particular embodiments of the compounds of formula (I) as described herein, n is 0 and R₂ is hydrogen.

In particular embodiments of the compounds of formula (I) as described, n is 0 and R₂ is methyl.

One embodiment of the disclosure provides compounds of formula (1) as described herein where ring A is a 5 or 6 membered heterocycloalkyl. In certain embodiments, ring A is pyrrolidinyl, azetidinyl, or piperidinyl.

In certain embodiments of the compounds of formula (I) as described herein, n is 0, R₂ is hydrogen, and ring A is pyrrolidinyl, azetidinyl, or piperidinyl. In certain embodiments of the compounds of formula (I) as described herein, n is 0. R₂ is C₁-C₄ alkyl, such as methyl, and ring A is pyrrolidinyl, azetidinyl, or piperidinyl.

In certain embodiments of the compounds of formula (I) as described herein, ring A is pyrrolidinyl. For example, in certain embodiments, ring A is of structure:

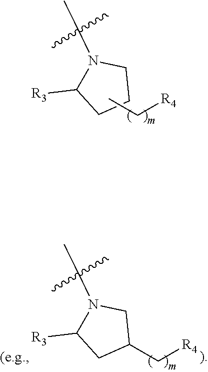

In certain other embodiments, ring A is an S-enantiomer of

structure:

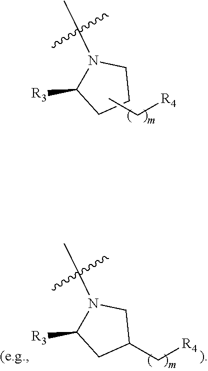

In certain other embodiments, ring A is a 2S, 4R-enatomer of structure

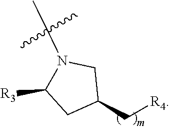

Another embodiment of the disclosure provides compounds of formula (I) as described herein where m is 1, 2, or 3. In certain embodiments, m is 1 or 2. In certain embodiments, m is 1.

In particular embodiments, compounds of formula (I) are wherein n is 0, R₂ is hydrogen, ring A is of structure: and

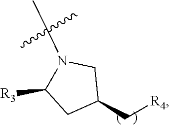

m is 1 or 2 (e.g., m is 1).

In particular embodiments, compounds of formula (I) are wherein n is 0, R₂ is C₁-C₄

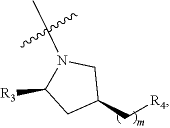

alkyl, such as methyl, ring A is of structure: and m is 1 or 2 (e.g., m is 1).

One embodiment of the disclosure provides compounds of formula (I) as described herein where R₃ is —CO₂R₅, —COR₅, —C(O)NR₅R₆, —CONH—OH, —SO₂R₅, —SO₂OR₅, or —SO₂NR₅R₆. In certain embodiments, R₃ is —CO₂R₅, —COR₅, —SO₂R₅, —SO₂OR₅, or —SO₂NR₅R₆. In certain embodiments, R₃ is —CO₂R₅, —SO₂R₅, —SO₂OR₅, or —SO₂NR₅R₆. In certain other embodiments, R₃ is —CO₂R₅, —COR₅, —C(O)NR₅R₆, or —CONH—OH. In certain other embodiments, R₃ is —CO₂R₆, —C(O)NR₅R₆, or —CONH—OH. In certain other embodiments. R₃ is —CO₂R₃ or —C(O)NR₅R₆. In certain other embodiments, R₃ is —CO₂R₅. In some embodiments, each R₃ is independently hydrogen or methyl, and each R₆ is independently hydrogen or methyl. In certain embodiments of the compounds of formula (I) as described herein R₃ is —CO₂H.

In certain embodiments of the compounds of formula (I) as described herein each R₃ is independently hydrogen or methyl, and each R₆ is independently hydrogen or methyl.

In example embodiments, R₃ is —C(O)H, —C(O)CH₃, —C(O)C₂H₅, —C(O)OCH₃, —C(O)OC₂H₆, —C(O)OH, —C(O)NH₂, —C(O)NHCH₃, —C(O)NCH₃CH₃, —S(O)CH₃, —S(O)C₂H₆, —S(O)2CH₃, —S(O)CHE, —S(O)OH, —S(O)OH, —S(O)OCH₃, or —S(O)OCH6. In certain embodiments, compounds of formula (I) as described here are where R₃ is —C(O)OCH₃, —C(O)OCH₆, —C(O)OH, —C(O)NH₂, —C(O)NHCH₃, —C(O)NCH&CH₃, —S(O)CH₃, or —S(O)CH₆.

In certain embodiments, compounds of formula (1) as described herein are where R₃ is —C(O)OH.

Another embodiment of the disclosure provides compounds of formula (1) as described herein where R₄ is selected from —C(O)NR₆R₇, —CO₂R₇, and —SO₂NR₆R₇. For example, in certain embodiments, R₃ is —C(O)NR₆R₇ or —SO₂NR₆R₇. In certain embodiments, R₄ is —C(O)NR₆R₇. In the compounds of formula (I) as described herein, R₆ is hydrogen or C₁-C₄ alkyl. For example, Re is hydrogen. In another example, R₃ is methyl.

In particular embodiments in the compounds of formula (I) ring A is of structure:

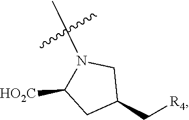

and R₄ is —C(O)NR₆R₇. For example, the compounds of formula (I) as described here are of formula:

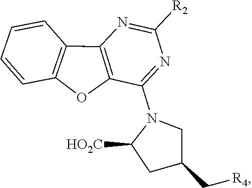

wherein R₂ is hydrogen or C₁-C₄ alkyl, such as methyl.

In one embodiment of the compounds of formula (I) as described herein, Re is hydrogen or C₁-C₄ alkyl. In certain embodiments, RE is hydrogen. In certain embodiments, Re is methyl.

Another embodiment of the disclosure provides compounds of formula (I) as described herein where R₇ is selected from the group consisting of aryl(C₀-C₁ alkyl) optionally substituted with one or more R₉,heteroaryl(C₀-C₁ alkyl) optionally substituted with one or more R₈, heterocyclyl(C₀-C₁ alkyl) optionally substituted with one or more R₈, and cycloalkyl(C₀-C₁ alkyl) optionally substituted with one or more R₈. In certain embodiments, R₇ is selected from the group consisting of aryl optionally substituted with one or more R₉, heteroaryl optionally substituted with one or more Rg, heterocyclyl optionally substituted with one or more R₆, and cycloalkyl optionally substituted with one or more R₈. In certain embodiments. R₇ is selected from the group consisting of phenyl optionally substituted with one or more R₉, 5 to 12 membered heteroaryl optionally substituted with one or more R₉, 5 to 12 membered heterocyclyl optionally substituted with one or more R₅, and C₃-C₈ cycloalkyl optionally substituted with one or more R₈. In certain other embodiments, R₂ is selected from the group consisting of phenyl optionally substituted with one or more R₉, 5 to 12 membered heteroaryl optionally substituted with one or more Ra, and C₃-C₈ cycloalkyl optionally substituted with one or more R₈. In certain other embodiments, R; is C₃-C₈ cycloalkyl optionally substituted with one or more R₃ (e.g., optionally substituted cyclopentane). In certain other embodiments, R₇ is phenyl optionally substituted with one or more R₃ or a 5 to 12 membered heteroaryl optionally substituted with one or more R₉. In certain other embodiments, R₇ is phenyl optionally substituted with one or more R₉. In certain other embodiments, R₇ is phenyl substituted with one or more R₉. In certain other embodiments, R₇ is 5 to 12 membered heteroaryl (e.g., pyridinyl, indolyl, indazolyl, benzothiazolyl, benzimidazolyl, quinolinyl, or isoquinolinyl) optionally substituted with one or more R₉. In certain embodiments. R₇ is bicyclic heteroaryl (e.g., indolyl, indazolyl, benzothiazolyl, benzimidazolyl, quinolinyl, or isoquinolinyl) optionally substituted with one or more R₉.

In one embodiment of the compounds of formula (1) as described herein, R₇ is phenyl, pyridinyl, indolyl, indazolyl, benzothiazolyl, benzimidazolyl, quinolinyl, or isoquinolinyl, each optionally substituted with one or more R₉, or cyclopentanyl optionally substituted with one or more R₈.

In one embodiment of the compounds of formula (1) as described herein, R₇ is phenyl substituted with one or more R₉; pyridinyl substituted with one or more R₉; indolyl, indazolyl, benzothiazolyl, benzimidazolyl, quinolinyl, or isoquinolinyl, each optionally substituted with one or more R₉, or cyclopentanyl substituted with one or more R₈.

In one embodiment of the compounds of formula (I) as described herein, each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl), —OH, C₁-C₆ alkoxy, C₁-C₆ haloalkoxy, —C(O)R₆, —C(O)OR₆, and —C(O)NR₆R₅, or two R₃ form an oxo. In certain embodiments, each Re is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl), —OH, C₁-C₄ alkoxy, and C₁-C₆ haloalkoxy, or two Re form an oxo. In certain embodiments, each R₃ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, and C₁-C₄ haloalkoxy. In certain other embodiments, each Re is independently selected from the group consisting of halogen. —NO₂, —CN, C₁-C₄ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, aryl-methyl-, heteroaryl, heteroaryl-methyl-, and heterocyclyl each optionally substituted with one or more R₁₀. In certain other embodiments, each R₉ is independently selected from the group consisting of halogen, C₁-C₄ alkyl, C₁-C₄ haloalkyl. —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazolyl, pyridazinyl, imidazolyl, and 2-oxooxazolidinyl, each optionally substituted with one or more R₁₀.

In particular embodiments, the compounds of formula (I) as described herein are

wherein:
m is an integer of 1;
n is an integer of 0 or 1;
ring A represents a pyrrolidinyl, azetidinyl, or piperidinyl ring;
each R₁ is independently selected from C₁-C₃ alkyl, —OH, and C₁-C₃ alkoxy;
R₂ is hydrogen, C₁-C₄ alkyl, or C₁-C₄ haloalkyl;
R₃ is —CO₂R₆ or —C(O)NR₅R₆; and
R₄ is —C(O)NR₆R₇ or —SONR₆R₇,
wherein
R₅ is hydrogen or C₁-C₄ alkyl;
R₆ is hydrogen, C₁-C₄ alkyl, or C₁-C₄ haloalkyl;
R₇ is selected from the group consisting of phenyl optionally substituted with one or more R₉, 5 to 12 membered heteroaryl optionally substituted with one or more R₉, 5 to 12 membered heterocyclyl optionally substituted with one or more R₅, and C₃-C₈ cycloalkyl optionally substituted with one or more R₈;
each Re is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₃ form an oxo; and
each Ry is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazolyl, pyridazinyl, imidazolyl, and 2-oxooxazolidinyl, each optionally substituted with one or more R₁₀.

In particular embodiments, the compounds described herein are of formula:

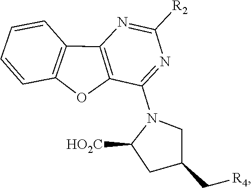

wherein
R₂ is hydrogen, C₁-C₄ alkyl, or C₁-C₄ haloalkyl;
R₄is —C(O)NR₆R₇ or —SO₂NR₆R₇, wherein
R₆ is hydrogen, C₁-C₄ alkyl, or C₁-C₄ haloalkyl;
R₇ is selected from the group consisting of phenyl optionally substituted with one or more R₉, 5 to 12 membered heteroaryl optionally substituted with one or more R₈, 5 to 12 membered heterocyclyl optionally substituted with one or more R₅, and C₃-C₈ cycloalkyl optionally substituted with one or more R₈;
each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₃ form an oxo; and
each R₉ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazolyl, pyridazinyl, imidazolyl, and 2-oxooxazolidinyl, each optionally substituted with one or more Rio.

In particular embodiments, the compounds of formula (I) as described herein are wherein:

m is an integer of 1:
n is an integer of 0 or 1;
ring A represents a pyrrolidinyl, azetidinyl, or piperidinyl ring:
each R₁ is independently selected from C₁-C₃ alkyl, —OH, and C₁-C₃ alkoxy;
R₂ is hydrogen, C₁-C₄ alkyl, or C₁-C₄ haloalkyl;
R₃ is —COzR5 or —C(O)NR₅R₆; and
R₄ —C(O)NNR₆R₇ or —SO₂NR₆R₇.
wherein
R₅ is hydrogen or C₁-C₄ alkyl;
R₆ is hydrogen, C₁-C₄ alkyl, or C₁-C₄ haloalkyl;
R₇ is selected from the group consisting of phenyl optionally substituted with one or more R₉, pyridinyl, indolyl, indazolyl, benzothiazolyl, benzimidazolyl, quinolinyl, or isoquinolinyl, each optionally substituted with one or more R₉, and cyclopentyl optionally substituted with one or more R₈;
each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₃ form an oxo; and
each R₉ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazolyl, pyridazinyl, imidazolyl, and 2-oxooxazolidinyl, each optionally substituted with one or more Rio.

In particular embodiments, the compounds described herein are of formula:

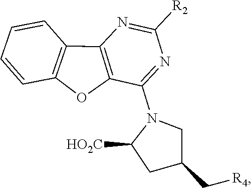

wherein
m is an integer of 1;
n is an integer of 0 or 1;
ring A represents a pyrrolidinyl, azetidinyl, or piperidinyl ring;
each R₁ is independently selected from C₁-C₄ alkyl, —OH, and Ci-C3 alkoxy;
R₂ is hydrogen, C₁-C₄ alkyl, or C₁-C₄ haloalkyl;
R₃ is —CO₂R. or —C(O)NR.R5; and
R₄ is —C(O)NNR₆R₇ or —SO₂NR&R;,
wherein
R₃ is hydrogen or C₁-C₄ alkyl;
RÅ is hydrogen, C₁-C₄ alkyl, or C₁-C₄ haloalkyl;
R₇ is selected from the group consisting of phenyl optionally substituted with one or more Ra, pyridinyl, indolyl, indazolyl, benzothiazolyl, benzimidazolyl, quinolinyl, or isoquinolinyl, each optionally substituted with one or more Ra, and cyclopentyl optionally substituted with one or more R₈;
each R₃ is independently selected from the group consisting of halogen, —NO₂, —CN, C₁-C₆ alkyl, C₁-C₆ haloalkyl, —NH₂, —NH(C₁-C₆ alkyl). —N(C₁-C₆ alkyl)₂, —OH, C₁-C₆ alkoxy, and C₁-C₆ haloalkoxy, or two R₃ form an oxo; and
each R₆ is independently selected from the group consisting of halogen, C₁-C₄ alkyl, C₁-C₄ haloalkyl. —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl. piperazinyl, pyrazolyl, pyrimidinyl, indazolyl, pyridazinyl, imidazolyl, and 2-oxooxazolidinyl, each optionally substituted with one or more R₁₀.

In certain embodiments, compounds of formula (I) as otherwise described herein are one of compounds listed in Example 2.

In certain embodiments, disclosure also provides a cGAS inhibitor compound (e.g., a compound of formula (I) as discussed above) having an IC₅₀ in the presence of Mn²⁺ that is at least 5-fold more than the IC₅₀ of the compound in otherwise identical conditions but lacking Mn²⁺.

In one embodiment of the disclosure, the compound as otherwise disclosed herein (e.g., a compound of formula (I), or recited in Example 2) is in the form of an N-oxide.

In one embodiment of the disclosure, the compound as otherwise disclosed herein (e.g., a compound of formula (1), or recited in Example 2) is in the form of a pharmaceutically acceptable salt. The person of ordinary skill in the art will appreciate that a variety of pharmaceutically-acceptable salts may be provided, as described in additional detail below. The person of ordinary skill in the art will appreciate that the phrase “optionally in the form of a pharmaceutically acceptable salt or N-oxide, or a solvate or hydrate” includes compounds in the form of a pharmaceutically acceptable salt of an N-oxide. But in certain embodiments as described above, the compound is not in the form of a pharmaceutically acceptable salt. Thus, in one embodiment, the compound as otherwise disclosed herein is in the form of the base compound.

In one embodiment of the disclosure, the compound as otherwise disclosed herein (e.g., a compound of formula (I), or recited in Example 2) is in the form of solvate or hydrate. The person of ordinary skill in the art will appreciate that a variety of solvates and/or hydrates may be formed. The person of ordinary skill in the art will appreciate that the phrase “optionally in the form of a pharmaceutically acceptable salt or N-oxide, or a solvate or hydrate” includes compounds in the form of solvates and hydrates of base compounds. pharmaceutically acceptable salts and N-oxides as described above. But in certain embodiments as described above, the compound is not in the form of a solvate or hydrate.

In one embodiment of the disclosure, the compound as otherwise disclosed herein (e.g., a compound of formula (I), or recited in Example 2) is in the form of an N-oxide. But in certain embodiments as described above, the compound is not in the form of an N-oxide.

### Therapeutics Applications

The inventors have determined that, in certain embodiments, the presently described compounds can inhibit cGAS. Accordingly, one aspect of the disclosure provides a method for treating or preventing inappropriate activation of a type I interferon (IFN) response in a subject in need thereof, the method comprising administering to the subject an effective amount of one or more compounds of the disclosure as described herein (e.g., a compound of formula (I) or those provided in Example 3) or a pharmaceutical composition of the disclosure as described herein. In certain embodiments of the methods as otherwise described herein, the inappropriate activation of a type I IFN comprises an autoimmune disorder. In certain such embodiments, the autoimmune disorder is Aicardi-Goutieres Syndrome, retinal vasculopathy with cerebral leukodystropy, lupus erythematosus, scleroderma, or Sjögren's syndrome.

The disclosure also provides methods of treating an autoimmune disorder. Such method includes administering to a subject in need of such treatment an effective amount of one or more compounds of the disclosure as described herein or a pharmaceutical composition of the disclosure as described herein.

Many different autoimmune disorders can be treated with compounds and compositions of the disclosure. Autoimmune disorder particularly suitable to be treated by the methods of the disclosure include, but are not limited to, Aicardi-Goutieres Syndrome, retinal vasculopathy with cerebral leukodystropy, lupus erythematosus, scleroderma, and Sjögren's syndrome.

The compounds and compositions of the disclosure as described herein may also be administered in combination with one or more secondary therapeutic agents. Thus, in certain embodiment, the method also includes administering to a subject in need of such treatment an effective amount of one or more compounds of the disclosure as described herein (e.g., a compound of formula (I) or those provided in Example 3) or a pharmaceutical composition of the disclosure as described herein and one or more secondary therapeutic agents.

“Combination therapy,” in defining use of a compound of the present disclosure and another therapeutic agent, is intended to embrace administration of each agent in a sequential manner in a regimen that will provide beneficial effects of the drug combination (e.g., the compounds and compositions of the disclosure as described herein and the secondary therapeutic agents can be formulated as separate compositions that are given sequentially), and is intended as well to embrace co-administration of these agents in a substantially simultaneous manner, such as in a single capsule having a fixed ratio of these active agents or in multiple or a separate capsules for each agent. The disclosure is not limited in the sequence of administration: the compounds of and compositions of the disclosure may be administered either prior to or after (i.e., sequentially), or at the same time (i.e., simultaneously) as administration of the secondary therapeutic agent.

In certain embodiments, the secondary therapeutic agent may be administered in an amount below its established half maximal inhibitory concentration (ICso). For example, the secondary therapeutic agent may be administered in an amount less than 1% of, e.g., less than 10%, or less than 25%, or less than 50%, or less than 75%, or even less than 90% of the inhibitory concentration (IC₅₀).

### Pharmaceutical Compositions

In another aspect, the present disclosure provides compositions comprising one or more of compounds as described above with respect to formula (I) and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds. In certain embodiments, the composition may include one or more antibiotic compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, membrane stabilizers. 5LO inhibitors, leukotriene synthesis and receptor inhibitors, inhibitors of IgE isotype switching or IgE synthesis, IgG isotype switching or lgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating. emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle. including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant. e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an ICso of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Definitions

The following terms and expressions used herein have the indicated meanings.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C₆alkyl indicate the same functionality; similarly arylalkyl and -alkylaryl indicate the same functionality.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy. butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂ —, —CH₂CH₂CHC(CH₃)—, and—CH₂CH(CH₂CH₃)CH₂ —.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to. azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl. 2.3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means -CI, -Br, -I or -F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl. oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7.8-tetrahydroquinolin-2-yl, 5,6,7,8-tetrahydroquinolin-3-yl, 5,6,7.8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”. when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Subject” refers to a warm blooded animal such as a mammal, preferably a human, or a human child, which is afflicted with, or has the potential to be afflicted with one or more diseases and disorders described herein.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie. “Protective Groups in Organic Chemistry.” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1. Georg Thieme Verlag, Stuttgart 1974, in H .-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art. For example, the compounds of structural formula (I) can be prepared according to general procedures of the Examples and/or analogous synthetic procedures. One of skill in the art can adapt the reaction sequences of these Examples and general procedures to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### EXAMPLES

The compounds and the methods of the disclosure is illustrated further by the following examples, which are not to be construed as limiting the disclosure in scope or spirit to the specific procedures and compounds described in them.

### Example 1. General Synthetic Method for the Compounds of the Disclosure

All solvents were purchased from commercial suppliers and used without further purification. ¹H and ¹³C NMR spectra were recorded on a Varian Mercury 300 MHz or Bruker BioSpin spectrometer, at 400/500 MHz. Mass spectra were measured in the electrospray ionization (ESI) mode at an ionization potential of 70 eV with a liquid chromatography mass spectrometry (LC/MS). Purity of all final compounds (greater than 95%) was determined by an analytical high-performance liquid chromatography (HPLC).

Benzofuro[3,2-d]pyrimidine precursor, such as 4-chloro-2-methylbenzofuro[3,2-d]pyrimidine, 6, was prepared essentially according to the following procedure:

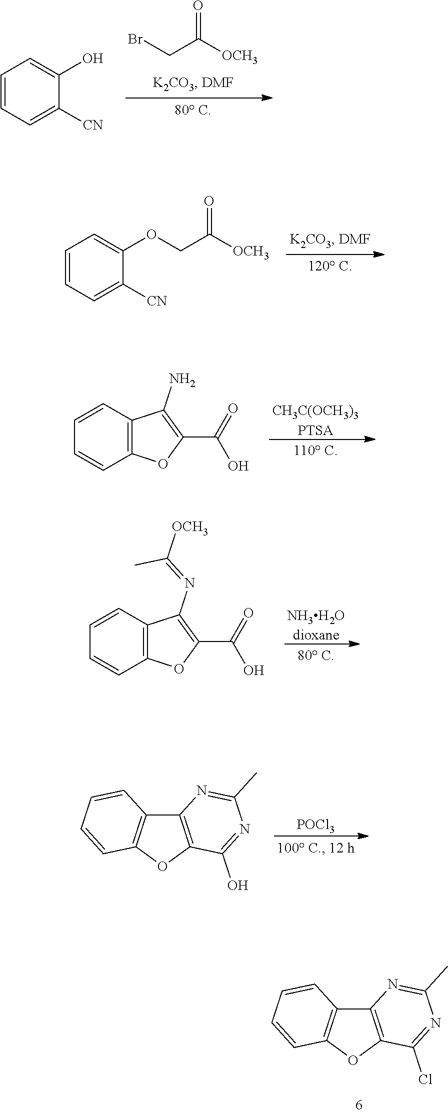

Benzofuro[3,2-d]pyrimidine precursor can be functionalized to arrive at compounds of formula (I) essentially according to the following procedure.

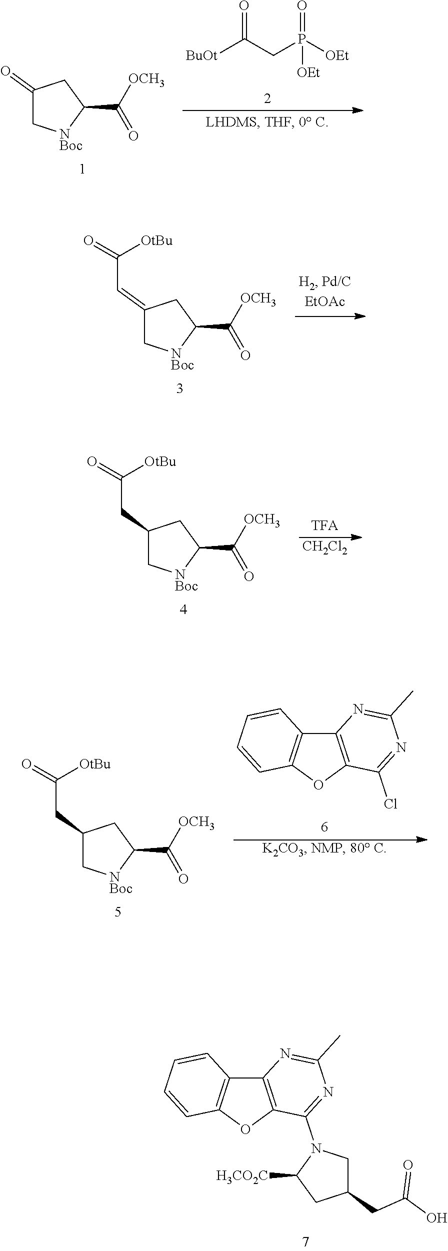

To a suspension of tert-butyl 2-(diethoxyphosphoryl)acetate (1) (34.2g/31.9 mL, 136 mmol) in anhydrous THF (100 mL) under nitrogen with vigorous stirring was added the LHMDS (45 mL of 1M/THF) dropwise at 0° C. After stirring at 0° C. for 30 min, 1-(tert-butyl) 2-methyl (S)-4-oxopyrrolidine-1,2-dicarboxylate (2) (30.0 g, 123 mmol) in THF (100 mL) was added dropwise. The resulting mixture was allowed to warm up to room temperature and stirring overnight. The reaction mixture was poured into saturated aq. NH₄Cl and extracted with EtOAc. The organics were washed sequentially with water, brine, and dried (Na₂SO₄). Filtration and concentration in vacuum gave a crude product, which was purified by Yamazen silica gel flash chromatography by using 0-20% EtOAc in Hexanes to afford 1-(tert-butyl) 2-methyl (S,E)-4-(2-(tert-butoxy)-2-oxoethylidene)pyrrolidine-1,2-dicarboxylate (3) (40 g, 95%) as an oil. LCMS [M+H]⁺ C₁₇H₂₇NO₆ 342.37.

A suspension of 1-(tert-butyl) 2-methyl (S)-4-(2-(tert-butoxy)-2-oxoethylidene)pyrrolidine-1,2-dicarboxylate (3) (40g, 120mmol), Pd/C (20%, 50% wetted) (19g, 18mmol) and EtOAc (300mL) was stirred overnight under H2 atmosphere. The reaction mixture was filtered, washed with EtOAc, and concentrated to provide compound 4 in quantitative yield as a colorless oil, which was used in the following step without further purification. LC-MS 344.26 (ES+); ¹H NMR (400 MHz, CDCl₃) δ 4.29 and 4.28 (two t, ratio=1.2:1, 1H), 3.82 (m, 1H), 3.71 and 3.72 (two s, ratio=2:1, 3H), 3.09 (m, 1H), 2.51 and 2.39 (two m, 2H+1H), 2.45 (m 1H), 1.66 (m, 1H), 1.55 and 1.44 (four s, 9H+9H): Rotameric ratio=˜1.5: 1.

To a solution of 1-(tert-butyl) 2-methyl (2S,4R)-4-(2-(tert-butoxy)-2-oxoethyl)pyrrolidine-1,2-dicarboxylate (4) (40 g, 116mmol) in DCM (300 mL), was added TFA (13.28 g. 116mmol) dropwise. After stirring at room temperature overnight, the reaction mixture was concentrated in vacuum to afford compound 5 (˜30g, 90% yield) as an oil, which was used in the following step without further purification. ¹H NMR (400 MHZ, CDCl₃) δ 4.46 (two d, 1H), 3.75 (s, 3H), 3.40 (dd, 1H), 2.92 (dd, 1H), 2.45 ˜2.6 (set of m, overlapped with solvent, 4H). 1.68 (two t. J=8 Hz, 1H); Ratio=93:7.

To a solution of methyl (2S,4R)-4-(2-(tert-butoxy)-2-oxoethyl)pyrrolidine-2-carboxylate (5) (TFA salt, 21.7g, 72.2mmol) in NMP (125mL), was added 4-chloro-2-methylbenzofuro[3,2-d]pyrimidine (11.3 g, 56 mmol) and K2CO3.(25 g, 180mmol). After the reaction mixture was stirred at 80° C. overnight, water and EtOAc were added and the layers were separated. The organics were washed sequentially with water, brine and dried (Na₂SO₄). Filtration and concentration in vacuum gave a crude product, which was purified by flash chromatography (silica gel, 0-50% EtOAc in PE) to afford the title compound 7 (9.5g, 51% yield). 1HNMR (400 MHz, DMSO) δ 8.05 (d. J=8 Hz, 1H), 7.77 (br, 0.5H), 7.67(t, 1.5H), 7.46 (two d. J=4, 8 Hz, 1H), 5.17 (br, 0.5H), 4.48 and 4.62 (two br, 1H), 4.26 (br, 0.5H), 3.68 (s, 3H), 3.57 (br, 1H). 2.68 (m, 1H), 2.48 (m, overlapped with DMSO, 3H+3H), 1.66 and 1.81 (two br. 1H); Vriation temperature (60° C.) δ 8.05 (d. J=8 Hz, 1H), 7.67 (dd and dt, 2H), 7.47 (two d, J=4, 8 Hz, 1H), 4.82 (br, 1H), 4.43 (br, 1H), 3.68 (s, 3H), 3.57 (br. 1H), 2.65 (m.1H+1H), 2.48 (m, overlapped with DMSO, 3H+2H), 1.75 (m, 1H); LC-MS 369.8 (ES+) 368.2 (ES−).

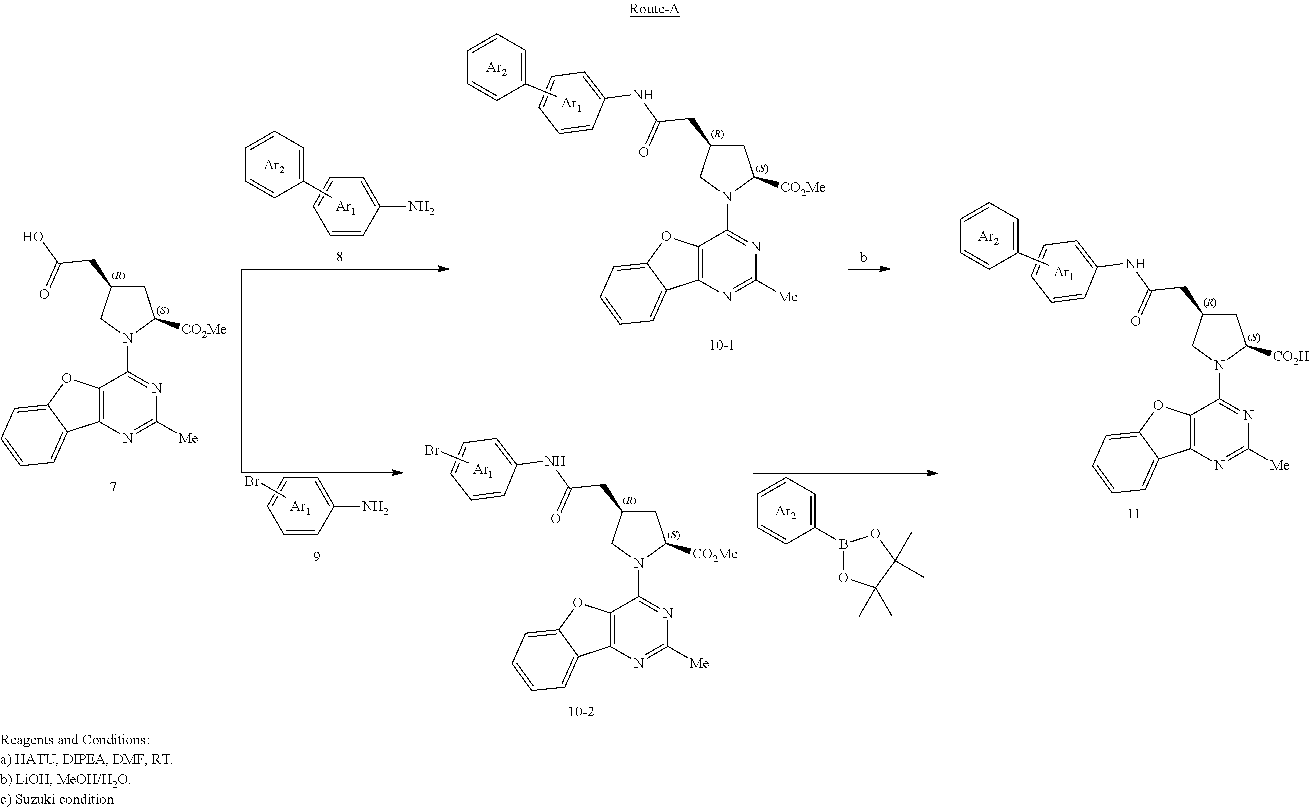

Synthesis of 10-1 or 10-2: To a stirred solution of carboxylic acid 7 (11 mmol, 1eq) in DMF (40 mL) was added DIEA (3eq), followed by HATU (1.1eq) in an ice water bath to stir at RT for 10 min. To the solution was added corresponding aniline 8 or 9 (1.1 eq) was added to stir at RT for 6 h. After work up, crude was purified by flash chromatography (MeOH: DCM =0:100 to 5: 95) to obtain ester 10-1 or 10-2 in good yield.

Synthesis of Target via Route-A: To a suspension of 10-1 (133.4 mg, 248.1 umol) in THF (3 mL) and water (1 mL) was added LiOH (25eq) to stir at RT for 4 h and to warm up at 37° C. for additional 4 h. After cooling to rt, the solution was acidify (PH ˜5) with 6N HCl to purify by prep HPLC to obtain 11.

Synthesis of Target via Route-B: To a suspension of 10-2 (1 eq), borate (1.5eq), potassium carbonate (2eq), and tetrakis(triphenylphosphine)palladium (0) (0.1eq) in dioxane (2 mL) and water (1 mL) was degassed and refill with Ar for 5 times. The resulting reaction mixture was heated to 90° C. for 2 h to become dark solution. To the solution was added activated carbon to stir for 15 min to filter. The filtrate was concentrated and purified by flash chromatography (MeOH: DCM=0:100 to 10: 90) to obtain ester.

The esters were hydrolyzed by the same method as of Route A. Synthesis of compounds 47, 48, 51, 59, and 60 (noted below) was performed as Route B.

### Example 2. The Compounds of the Disclosure

The following compounds were prepared substantially according to the procedures described above and procedures familiar to the person of ordinary skill in the art. Compound A is a comparative compound.

Compound A: (2S,4R)-1-(2-methylbenzofuro[3,2-d]pyrimidin-4-yl)-4-(2-oxo-2-(pyridine-4-ylamino)ethyl)pyrrolidine-2-carboxylic acid (BBL0100352)

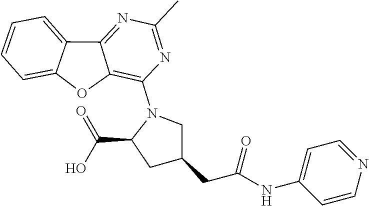 [SMILES: Cc1nc(N2C[C@@H](CC(=O)Nc3ccncc3)C[C@H]2C(=O)O)c2oc3ccccc3c2n1]

Compound 1: (2S,4R)-4-(2-((1H-indol-5-yl)amino)-2-oxyethyl)-1-(2-methylbenzofuro[3,2-d]pyrimidin-4-yl)pyrrolidine-2-carboxylic acid (BBL0100462)

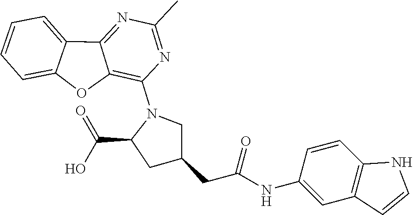 [SMILES: Cc1nc(N2C[C@@H](CC(=O)Nc3ccc4[nH]ccc4c3)C[C@H]2C(=O)O)c2oc3ccccc3c2n1]

Compound 2: (2S,4R)-4-(2-(cyclopentyl(methyl)amino)-2-oxoethyl)-1-(2-methylbenzofuro[3,2-d]pyrimidin-4-yl)pyrrolidine-2-carboxylic acid (BBL0100461)

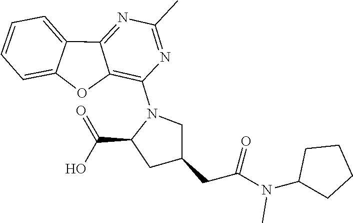 [SMILES: Cc1nc(N2C[C@@H](CC(=O)N(C)C3CCCC3)C[C@H]2C(=O)O)c2oc3ccccc3c2n1]

Compound 3: (2S,4R)-4-(2-((1H-indazol-5-yl)amino)-2-oxyethyl)-1-(2-methylbenzofuro[3,2-d]pyrimidin-4-yl)pyrrolidine-2-carboxylic acid (BBL0100459)

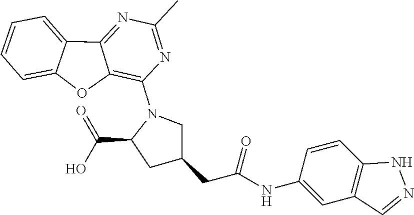 [SMILES: Cc1nc(N2C[C@@H](CC(=O)Nc3ccc4[nH]ncc4c3)C[C@H]2C(=O)O)c2oc3ccccc3c2n1]

Compound 4: (2S,4R)-4-(2-(benzo[d]thiazol-6-ylamino)-2-oxyethyl)-1-(2-methylbenzofuro[3,2-d]pyrimidin-4-yl)pyrrolidine-2-carboxylic acid (BBL0100458)

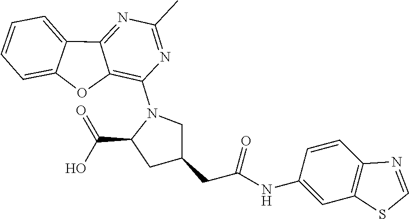 [SMILES: Cc1nc(N2C[C@@H](CC(=O)Nc3ccc4ncsc4c3)C[C@H]2C(=O)O)c2oc3ccccc3c2n1]

Compound 5: (2S,4R)-4-(2-([1,1′-biphenyl]-4-ylamino)-2-oxoethyl)-1-(2-methylbenzofuro[3,2-d]pyrimidin-4-yl)pyrrolidine-2-carboxylic acid (BBL0100455)

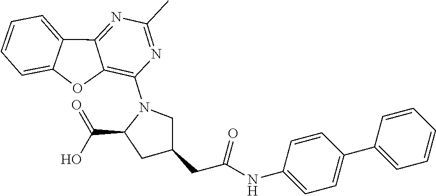 [SMILES: Cc1nc(N2C[C@@H](CC(=O)Nc3ccc(-c4ccccc4)cc3)C[C@H]2C(=O)O)c2oc3ccccc3c2n1]

¹HNMR (500 MHZ, DMSO-d₆) 6 12.4˜13.2 (br, 1H). 10.10 (s, 1H), 8.05 (br d, J=8 Hz, 1H), 7.76 (B of ABq, J=8 Hz, 2H), 7.71 (d and t, J=8 Hz, 6H), 7.43 (t, J=8 Hz, 3H), 7.31 (two t, J=8 Hz, 1H), 5.13 (br, 1H), 4.59-4.29 (m, 1H), 3.73 (br, 1H), 3.01-2.92 (m, 7H), 1.90 -1.74 (m, 1H); (ESI) m/z calcd for C₃₀H₂₆N₄O₄: 506.20. Found: 507.06 (M+H)⁺.

Compound 6: (2S,4R)-4-(2-((2-methoxypyridin-4-yl)amino)-2-oxoethyl)-1-(2-methylbenzofuro[3,2-d]pyrimidin-4-yl)pyrrolidine-2-carboxylic acid (BBL0100460)

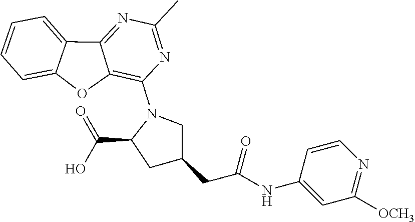 [SMILES: COc1cc(NC(=O)C[C@H]2C[C@@H](C(=O)O)N(c3nc(C)nc4c3oc3ccccc34)C2)ccn1]

Compound 7: (2S,4R)-1-(2-methylbenzofuro[3,2-d]pyrimidin-4-yl)-4-(2-oxo-2-((4-(piperidin-1-yl)phenyl)amino)ethyl)pyrrolidine-2-carboxylic acid (BBL-0100711)

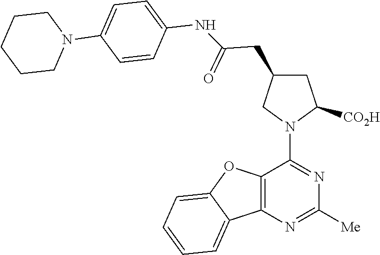 [SMILES: Cc1nc(N2C[C@@H](CC(=O)Nc3ccc(N4CCCCC4)cc3)C[C@H]2C(=O)O)c2oc3ccccc3c2n1]

¹HNMR (400 MHZ, DMSO-d₆) δ 12.83 (br. 1H), 10.16 (br, 1H), 8.14 (d. J=8 Hz, 1H), 7.88 (br, 1H), 7.75 (m, 4H), 7.55 (m, 1H), 7.40 (br, 1H), 5.24 (br, 1H), 4.68 (br m, 1H), 4.35-3.80 (br, 1H), 3.39 (br, 4H), 2.9-2.4 (m, overlapped with DMSO, 7H), 1.95 (br, overlapped, 1H), 1.80 (br, 4H), 1.60 (br, 2H): (ESI) m/z calcd for C₂₉H₃₁N₅O₄: 513.24. Found: 514.25 (M+H)⁺.

Compound 8: (2S,4R)-1-(2-methylbenzofuro[3,2-d]pyrimidin-4-yl)-4-(2-oxo-2-((4-(piperazin-1-yl)phenyl)amino)ethyl)pyrrolidine-2-carboxylic acid (BBL-0100716)

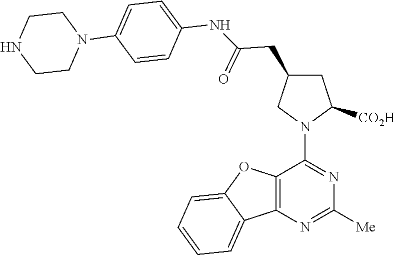 [SMILES: Cc1nc(N2C[C@@H](CC(=O)Nc3ccc(N4CCNCC4)cc3)C[C@H]2C(=O)O)c2oc3ccccc3c2n1]

¹HNMR (400 MHZ, DMSO-d₆) δ 10.84 (s, 1H), 8.66 (br, 1H), 8.08 (d, J=10 Hz, 1H), 7.79 (br, 1H), 7.68 (m, 1H), 7.49 (t. J=10 Hz, 3H), 6.96 (d. J=10 Hz, 2H), 5.15 (br, 1H), 4.60 (br d, 1H), 4.27-3.71 (br, overlapped, 1H), 3.25 (m, 1H), 2.4˜2.9 (m, overlapped with DMSO, 7H), 1.80 (br d, 1H); (ESI) m/z calcd for C₂₈H₃₀N₆O₄: 514.23. Found: 515.32 (M+H)⁺.

Compound 9: (2S,4R)-1-(2-methylbenzofuro[3,2-d]pyrimidin-4-yl)-4-(2-((4-(4-methylpiperazin-1-yl)phenyl)amino)-2-oxoethyl)pyrrolidine-2-carboxylic acid (BBL-0100717)

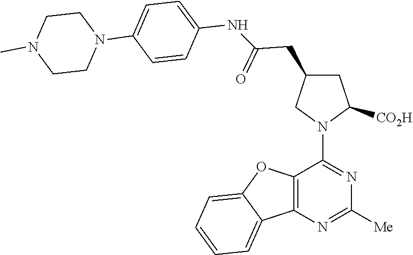 [SMILES: Cc1nc(N2C[C@@H](CC(=O)Nc3ccc(N4CCN(C)CC4)cc3)C[C@H]2C(=O)O)c2oc3ccccc3c2n1]

¹HNMR (500 MHZ, DMSO-d₆) δ 12.73 (br, 1H), 9.86 (s, 1H), 9.72 (br, 1H), 8.12 (d. J=10 Hz, 1H), 7.83 (br, 1H), 7.70 (m, 1H), 7.50 (t and B of ABq, J=10 Hz, 3H), 6.95 (A of ABq. J=10 Hz, 2H), 5.25 (br, 1H). 4.66-4.42 (br s. 1H), 3.75 (br, overlapped, 4H), 3.13-2.90 (m, 4H), 2.85 (s, 3H), 2.9-2.4 (m, overlapped with DMSO, 8H), 1.80 (br d, 1H). (ESI) m/z calcd for C₂₉H₃₂N₆O₄: 528.25. Found: 529.37 (M+H)⁺.

Compound 10: (2S,4R)-4-(2-((4-(1H-pyrazol-4-yl)phenyl)amino)-2-oxoethyl)-1-(2-methylbenzofuro[3,2-d]pyrimidin-4-yl)pyrrolidine-2-carboxylic acid (BBL-0100718)

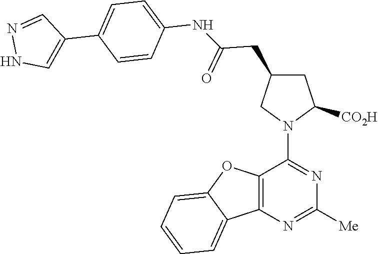 [SMILES: Cc1nc(N2C[C@@H](CC(=O)Nc3ccc(-c4cn[nH]c4)cc3)C[C@H]2C(=O)O)c2oc3ccccc3c2n1]

¹HNMR (500 MHZ, DMSO-d₆) δ 12.79 (br, 1H), 9.98 (s, 1H), 8.07 (d, J=5 Hz, 1H), 7.98 (s, 2H), 7.76 (br. 1H). 7.66 (m, 1H). 7.60 (d, J=10 Hz, 2H), 7.53 (d. J=10 Hz, 2H), 7.45 (t. 1H), 5.13 (br, 1H), 4.60-4.26 (br s. 1H), 3.71 (br, 1H), 2.9-2.4 (m, overlapped with DMSO, 7H), 1.80 (br d, 1H); (ESI) m/z calcd for C₂₇H₂₄N₆O₄: 496.19. Found: 497.36 (M+H)⁺.

Compound 11: (25,4R)-4-(2-((3-(1H-pyrazol-4-yl)phenyl)amino)-2-oxoethyl)-1-(2-methylbenzofuro[3,2-d]pyrimidin-4-yl)pyrrolidine-2-carboxylic acid (BBL-0100730)

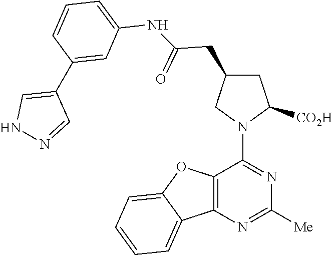 [SMILES: Cc1nc(N2C[C@@H](CC(=O)Nc3cccc(-c4cn[nH]c4)c3)C[C@H]2C(=O)O)c2oc3ccccc3c2n1]

¹HNMR (500 MHZ, DMSO-d₆) δ 12.80 (br, 1H), 10.00 (s, 1H), 8.05 (d. J=5 Hz, 1H). 7.95 (s. 2H), 7.79 (br, 2H), 7.66 (m, 1H), 7.45 (t, 2H), 7.28 (d. J=5 Hz, 2H), 5.13 (br, 1H), 4.60-4.26 (br s, 1H), 3.71 (br, 1H), 2.9-2.4 (m, overlapped with DMSO, 7H), 1.80 (br d, 1H); (ESI) m/z calcd for C₂₇H₂₄N₆O₄: 496.19. Found: 497.17 (M+H)⁺.

Compound 12: (2S,4R)-1-(2-methylbenzofuro[3,2-d]pyrimidin-4-yl)-4-(2-oxo-2-((4-(trifluoromethoxy)phenyl)amino)ethyl)pyrrolidine-2-carboxylic acid (BBL-0100724)

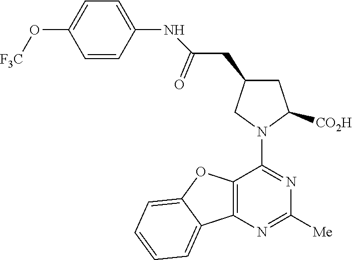 [SMILES: Cc1nc(N2C[C@@H](CC(=O)Nc3ccc(OC(F)(F)F)cc3)C[C@H]2C(=O)O)c2oc3ccccc3c2n1]

¹HNMR (500 MHz, DMSO-d₆) δ 12.67 (br, 1H), 10.10 (s, 1H), 8.06 (d, J=8 Hz, 1H), 7.77 (br, 1H), 7.72 (d, J=10 Hz, 2H), 7.65 (m, 1H), 7.45 (t, 1H), 7.31 (d. J=10 Hz, 2H), 5.12 (br, 1H), 4.60-4.25 (m. 1H), 3.72 (br, 1H), 2.4˜2.9 (m, overlapped with DMSO, 7H), 1.80 (br d. 1H); (ESI) m/z calcd for C₂₅H₂₁F₃N₄O₅: 514.15. Found: 514.94 (M+H)⁺.

Compound 13: (2S,4R)-4-(2-((3-methoxyphenyl)amino)-2-oxoethyl)-1-(2-methylbenzofuro[3,2-d]pyrimidin-4-yl)pyrrolidine-2-carboxylic acid (BBL-0100701)

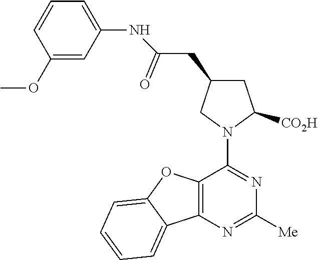 [SMILES: COc1cccc(NC(=O)C[C@H]2C[C@@H](C(=O)O)N(c3nc(C)nc4c3oc3ccccc34)C2)c1]

¹HNMR (400 MHZ, DMSO-d₆) δ 12.40 (br. 1H), 9.99 (s. 1H), 8.06 (d, J=8 Hz, 1H), 7.80 (br, 1H), 7.67 (d. J=8 Hz, 1H), 7.45 (t, 1H), 7.32 (s, 1H), 7.18 (m, 1H), 7.13 (d, 1H), 6.63 (dt, 1H), 5.10 (br. 1H), 4.56-4.26 (br s, 1H), 3.72 (s, overlapped, 4H), 2.9-2.4 (m, overlapped with DMSO, 7H), 1.80 (br d, 1H): (ESI) m/z calcd for C₂₅H₂₄N₄O₅: 460.17. Found: 461.9 (M+H)⁺.

Compound 14: (28.4R)-4-(2-((2-methoxyphenyl)amino)-2-oxoethyl)-1-(2-methylbenzofuro[3,2-d]pyrimidin-4-yl)pyrrolidine-2-carboxylic acid (BBL-0100726)

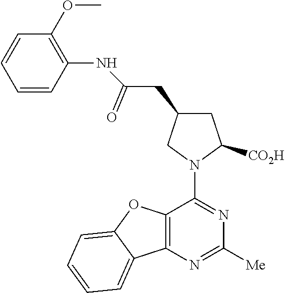 [SMILES: COc1ccccc1NC(=O)C[C@H]1C[C@@H](C(=O)O)N(c2nc(C)nc3c2oc2ccccc23)C1]

¹HNMR (400 MHZ, DMSO-d₆) δ 9.19 (br, 1H), 8.05 (d, J=8 Hz, 1H), 7.77 (br, 1H). 7.65 (m, 1H), 7.45 (t, 1H), 7.1-7.0 (m, 2H), 6.92 (dt, J=1.4 Hz, 1H), 5.11 (br, 1H), 4.59 -4.28 (m, 1H), 3.85 (s, 3H), 3.69 (br, 1H). 2.9-2.4 (m, overlapped with DMSO, 7H), 1.80 (br d, 1H). (ESI) m/z calcd for C₂₅H₂₄N₄O₅: 460.17. Found: 461.01 (M+1)⁺.

Compound 15: (2S,4R)-1-(2-methylbenzofuro[3,2-d]pyrimidin-4-yl)-4-(2-oxo-2-(p-tolylamino)ethyl)pyrrolidine-2-carboxylic acid (BBL-0100702)

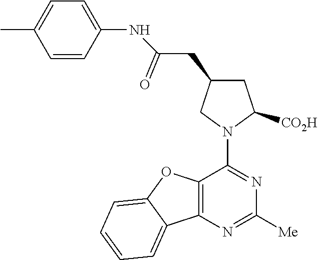 [SMILES: Cc1ccc(NC(=O)C[C@H]2C[C@@H](C(=O)O)N(c3nc(C)nc4c3oc3ccccc34)C2)cc1]

¹HNMR (400 MHZ, DMSO-d₆) δ 13.10 (br, 1H), 9.92(s, 1H), 8.14 (d. 1H), 7.78 (br, 1H), 7.76 (m, 1H), 7.56 (t, 1H), 7.49 (B of ABq, J=8 Hz, 2H), 7.10 (A of ABq, J=8 Hz, 2H), 5.13 (br, overlapped with H₂O, 1H), 4.68 (br d, 1H), 4.38 (br s, 0.5H), 3.79 (br, 0.5H), 3.42 (m, 0.5H), 2.4-2.9 (m, overlapped with DMSO, 3H+3H+1.5H), 2.24 (s, 3H), 1.76 and 2.00 (two br, 1H); (ESI) m/z calcd for C₂₅H₂₄N₄O₄: 444.18. Found: 444.94 (M+1)⁺.

Compound 16: (28.4R)-1-(2-methylbenzofuro[3,2-d]pyrimidin-4-yl)-4-(2-oxo-2-(m-tolylamino)ethyl)pyrrolidine-2-carboxylic acid (BBL-0100703)

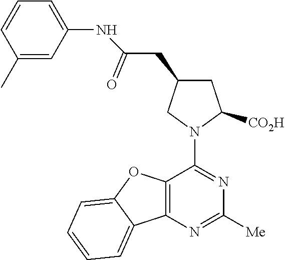 [SMILES: Cc1cccc(NC(=O)C[C@H]2C[C@@H](C(=O)O)N(c3nc(C)nc4c3oc3ccccc34)C2)c1]

¹HNMR (400 MHZ, DMSO-d₆) δ 13.05 (br, 1H), 9.94 (s, 1H), 8.15 (d, 1H), 7.89 (br, 1H), 7.76 (m, 1H), 7.56 (t, 1H), 7.46 (s, 1H), 7.38 (br d, 1H), 7.19 (t, 1H), 6.86 (d, 1H), 5.27 (br, 1H), 4.70-4.39 (br s. 1H), 3.79-3.42 (m, 1H), 2.9-2.4 (m, overlapped with DMSO, 7H) . 2.27 (s, 3H), 2.00-1.76 (m, 1H); (ESI) m/z calcd for C₂₅H₂₄N₄O₄: 444.18. Found: 444.94 (M+1)⁺.

Compound 17: (2S,4R)-1-(2-methylbenzofuro[3,2-d]pyrimidin-4-yl)-4-(2-oxo-2-(o-tolylamino)ethyl)pyrrolidine-2-carboxylic acid (BBL-0100725)

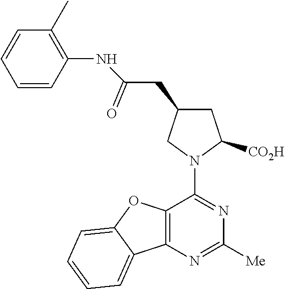 [SMILES: Cc1nc(N2C[C@@H](CC(=O)Nc3ccccc3C)C[C@H]2C(=O)O)c2oc3ccccc3c2n1]

¹HNMR (400 MHZ, DMSO-d₆) δ 12.69 (br, 1H), 9.39 (br, 1H), 8.05 (d, J=8 Hz, 1H). 7.75 (br, 1H), 7.66 (m, 1H), 7.46 (t, 1H), 7.40 (d, 1H), 7.20 (m, 2H), 7.09 (m, 1H), 5.13 (br, 1H), 4.62 -4.28 (m, 1H), 3.74 (br, 1H), 2.9-2.4 (m, overlapped with DMSO, 7H), 2.24 (s, 3H), 1.80 (br d. 1H); (ESI) m/z calcd for C₂₅H₂₄N₄O₄: 444.18. Found: 445.01 (M+1)⁺.

Compound 18: (2S,4R)-4-(2-((4-fluorophenyl)amino)-2-oxoethyl)-1-(2-methylbenzofuro[3,2-d]pyrimidin-4-yl)pyrrolidine-2-carboxylic acid (BBL-0100704)

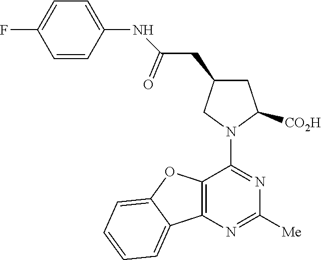 [SMILES: Cc1nc(N2C[C@@H](CC(=O)Nc3ccc(F)cc3)C[C@H]2C(=O)O)c2oc3ccccc3c2n1]

¹HNMR (400 MHZ, DMSO-d₆) δ 12.72 (br, 1H), 10.06 (s, 1H), 8.12 (d. J=8 Hz, 1H). 7.80 (br, 1H), 7.7-7.5 (m, 3H), 7.48 (t, 1H), 7.14 (d and t, 2H), 5.15 (br, 1H), 4.56-4.28 (m, 1H), 3.72 (br, overlapped, 1H), 2.9-2.4 (m, overlapped with DMSO, 7H), 1.85 (m, 1H); (ESI) m/z calcd for C₂₄H₂₁FN₄O₄: 448.50. Found: 449.91 (M+1)⁺.

Compound 19: (2S,4R)-4-(2-((3-fluorophenyl)amino)-2-oxoethyl)-1-(2-methylbenzofuro[3,2-d]pyrimidin-4-yl)pyrrolidine-2-carboxylic acid (BBL-0100719)

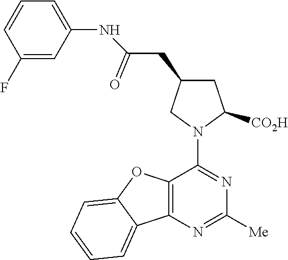 [SMILES: Cc1nc(N2C[C@@H](CC(=O)Nc3cccc(F)c3)C[C@H]2C(=O)O)c2oc3ccccc3c2n1]

¹HNMR (500 MHz, DMSO-d₆) δ 13.08 (br, 1H), 10.23 (s, 1H), 8.12 (d. J=10 Hz, 1H), 7.85 (br, 0.5H), 7.74 (m, 1.5H), 7.63 (d, J=10 Hz, 1H), 7.54 (t, 1H), 7.31 (m, 2H), 6.86 (m 1H), 5.28 (br, 1H), 4.67-4.34 (m, 1H), 3.71 (br, overlapped, 1H). 2.9-2.4 (m, overlapped with DMSO, 7H), 1.80 (br d. 1H); (ESI) m/z calcd for C₂₄H₂₁FN₄O₄: 448.50. Found: 449.91 (M+1)⁺.

Compound 20: (2S,4R)-4-(2-((2-fluorophenyl)amino)-2-oxoethyl)-1-(2-methylbenzofuro[3,2-d]pyrimidin-4-yl)pyrrolidine-2-carboxylic acid (BBL-0100720)

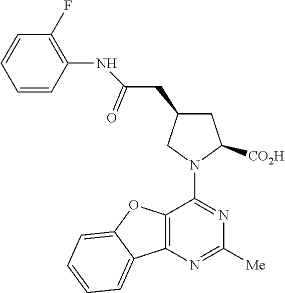 [SMILES: Cc1nc(N2C[C@@H](CC(=O)Nc3ccccc3F)C[C@H]2C(=O)O)c2oc3ccccc3c2n1]

¹HNMR (500 MHZ, DMSO-d₆) δ 12.70 (br, 1H), 9.80 (s, 1H), 8.06 (d, J=8 Hz, 1H), 7.87 (m, 1H), 7.78 (br, 1H), 7.66 (m, 1H), 7.46 (t, 1H), 7.25 (m, 1H), 7.16 (d and t, 2H), 5.15 (br, 1H), 4.60-4.28 (m. 1H), 3.71 (br, overlapped, 1H), 2.9-2.4 (m, overlapped with DMSO, 7H), 1.80 (br d, 1H): (ESI) m/z calcd for C₂₄H₂₁FN₄O₄: 448.50. Found: 449.91 (M+1)⁺.

Compound 21: (28.4R)-4-(2-((4-chlorophenyl)amino)-2-oxoethyl)-1-(2-methylbenzofuro[3,2-d]pyrimidin-4-yl)pyrrolidine-2-carboxylic acid (BBL-0100713)

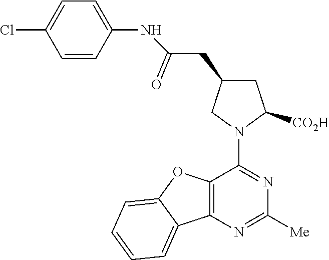 [SMILES: Cc1nc(N2C[C@@H](CC(=O)Nc3ccc(Cl)cc3)C[C@H]2C(=O)O)c2oc3ccccc3c2n1]

¹HNMR (500 MHZ, DMSO-d:) δ 12.66 (br, 1H), 10.14(s, 1H), 8.06 (d, J=8 Hz, 1H), 7.77 (br, 1H), 7.67 (d, J=8 Hz, 3H), 7.45 (t, J=4 Hz, 1H), 7.35 (d. J=8 Hz, 2H), 5.10 (br, 1H), 4.56-4.24 (m, 1H), 3.69 (br, 1H), 2.65 (overlapped, 1H), 2.48 (m, overlapped with DMSO, 6H), 1.80 (br d, 1H) ; (ESI) m/z calcd for C₂₄H₂₁ClN₄O₄: 464.13. Found: 465.04 (M+1)⁺.

Compound 22: (2S,4R)-4-(2-((3-chlorophenyl)amino)-2-oxoethyl)-1-(2-methylbenzofuro[3,2-d]pyrimidin-4-yl)pyrrolidine-2-carboxylic acid (BBL-0100705)

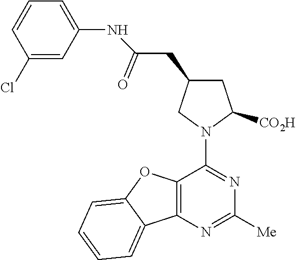 [SMILES: Cc1nc(N2C[C@@H](CC(=O)Nc3cccc(Cl)c3)C[C@H]2C(=O)O)c2oc3ccccc3c2n1]

¹HNMR (400 MHZ, DMSO-d₆) 6 12.69 (br, 1H), 10.21 (s. 1H), 8.09 (d, J=8 Hz, 1H), 7.84 (m, 2H), 7.79 (dd, 1H), 7.52 (t, 1H), 7.46 (d, 1H), 7.35 (t, 1H), 7.11(d, 1H), 5.19 (br, 1H), 4.64-4.32 (m, 1H), 3.74 (br, overlapped, 1H), 2.9-2.4 (m, overlapped with DMSO, 7H). 1.85 (br d. 1H); (ESI) m/z calcd for C₂₄H₂₁ClN₄O₄: 464.13. Found: 465.24 (M+1)⁺.

Compound 23: (28.4R)-4-(2-((2-chlorophenyl)amino)-2-oxoethyl)-1-(2-methylbenzofuro[3,2-d]pyrimidin-4-yl)pyrrolidine-2-carboxylic acid (BBL-0100706)

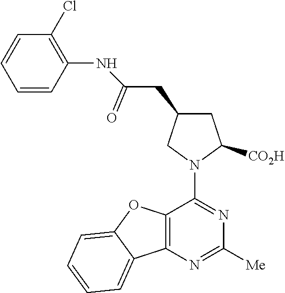 [SMILES: Cc1nc(N2C[C@@H](CC(=O)Nc3ccccc3Cl)C[C@H]2C(=O)O)c2oc3ccccc3c2n1]

¹HNMR (400 MHZ, CD₃OD) 8 8.13 (d, J=8 Hz, 1H), 7.73 (m, 3H), 7.40˜7.60 (m. 2H), 7.34 (t, 1H), 7.22 (t, 1H), 5.20 (br, 1H), 4.9-4.51 (m, 1H), 3.94 (m, 1H), 3.0-2.5 (m, 7H), 2.00 (br d. 1H); (ESI) m/z calcd for C₂₄H₂₁ClN₄O₄: 464.13. Found: 465.19 (M+1)⁺.

Compound 24: (28.4R)-1-(2-methylbenzofuro[3,2-d]pyrimidin-4-yl)-4-(2-oxo-2-((4-(trifluoromethyl)phenyl)amino)ethyl)pyrrolidine-2-carboxylic acid (BBL-0100721)

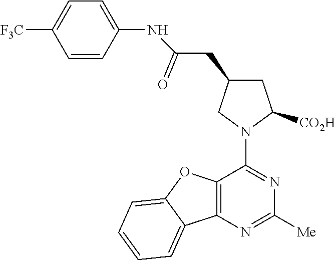 [SMILES: Cc1nc(N2C[C@@H](CC(=O)Nc3ccc(C(F)(F)F)cc3)C[C@H]2C(=O)O)c2oc3ccccc3c2n1]

¹HNMR (500 MHZ, CD₃OD) δ 12.59 (br, 1H), 8.13 (d. J=10 Hz, 1H), 7.85-7.65 (m, 4H), 7.60-7.50(m, 3H), 5.55-5.25 (br, 1H), 4.52-4.06 (m, 1H)), 3.90-3.61 (m, 1H), 2.91 -2.50 (m, 7H), 2.42 -1 1.95 (m, 1H): (ESI) m/z calcd for C₂₅H₂₁F₃N₄O₄: 498.46. Found: 500.02 (M+1)⁺.

Compound 25: (2S,4R)-1-(2-methylbenzofuro[3,2-d]pyrimidin-4-yl)-4-(2-oxo-2-((3-(trifluoromethyl)phenyl)amino)ethyl)pyrrolidine-2-carboxylic acid (BBL-0100712)

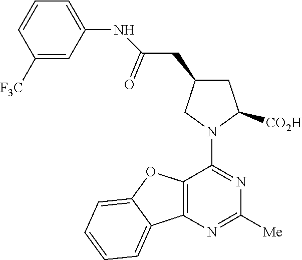 [SMILES: Cc1nc(N2C[C@@H](CC(=O)Nc3cccc(C(F)(F)F)c3)C[C@H]2C(=O)O)c2oc3ccccc3c2n1]

¹HNMR (400 MHZ, DMSO-d₆) δ 12.89-12.59 (br, 1H), 10.32(s, 1H), 8.12 (s, 1H), 8.06 (d. J=8 Hz, 1H), 7.79 (m, 1H), 7.67 (d. J=8 Hz, 2H), 7.57 (t, J=8 Hz, 1H), 7.45 (t, J=4 Hz, 1H). 7.40 (d. J=8 Hz, 1H), 5.13 (br, 1H), 4.61-4.27 (m, 1H), 3.73 (br, 1H), 2.9-2.4 (m, overlapped with DMSO, 7H), 1.80 (br d, 1H); (ESI) m/z calcd for C₂₅H₂₁F₃N₄O₄: 498.46. Found: 500.12 (M+1)⁺.

Compound 26: (2S,4R)-4-(2-((3,4-dichlorophenyl)amino)-2-oxoethyl)-1-(2-methylbenzofuro[3,2-d]pyrimidin-4-yl)pyrrolidine-2-carboxylic acid (BBL-0100710)

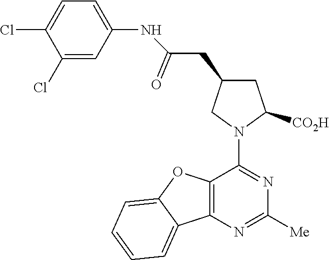 [SMILES: Cc1nc(N2C[C@@H](CC(=O)Nc3ccc(Cl)c(Cl)c3)C[C@H]2C(=O)O)c2oc3ccccc3c2n1]

¹HNMR (400 MHz, DMSO-d₆) δ 12.62 (br, 1H). 10.31(s, 1H), 8.06 (d, J=8 Hz, 1H), 8.02 (d. J=4 Hz, 1H), 7.78-7.60 (br, 2H), 7.55 (d, J=12 Hz, 1H), 7.50-7.40 (m, 1H+1H). 5.10 (br, 1H), 4.56-4.24 (m, 1H), 3.71 (br. 1H), 2.65 (overlapped, 1H), 2.48 (m, overlapped with DMSO, 6H). 1.75 (br d, 1H); (ESI) m/z calcd for C₂₄H₂₀Cl₂N₄O₄: 498.09. Found: 498.91 (M+1)⁺.

Compound 27: (2S,4R)-4-(2-((4-ethynylphenyl)amino)-2-oxoethyl)-1-(2-methylbenzofuro[3,2-d]pyrimidin-4-yl)pyrrolidine-2-carboxylic acid (BBL-0100709)

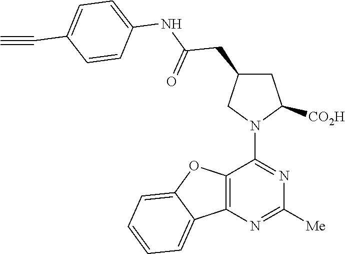 [SMILES: C#Cc1ccc(NC(=O)C[C@H]2C[C@@H](C(=O)O)N(c3nc(C)nc4c3oc3ccccc34)C2)cc1]

¹HNMR (400 MHZ, DMSO-d₆) δ 12.77 (br, 1H), 10.20 (s, 1H), 8.12 (d, J=8 Hz, 1H), 7.87 (br, 1H), 7.75 (m, 1), 7.63 (B of ABq, J=8 Hz, 2H), 7.55 (t, J=8 Hz, 1H), 7.42 (A of ABq. J=8 Hz, 2H), 5.21 (br, 1H), 4.66-4.36 (m, 1H), 4.07 (s, 1H), 3.78-3.43 (m, 1H), 2.9 -2.4 (br m. 7H), 1.76 and 1.93 (m, 1H); (ESI) m/z calcd for C₂₆H₂₂N₄O₄: 454.16. Found: 455.12 (M+1)⁺.

Compound 28: (28.4R)-4-(2-((1H-indazol-4-yl)amino)-2-oxoethyl)-1-(2-methylbenzofuro[3,2-d]pyrimidin-4-yl)pyrrolidine-2-carboxylic acid (BBL-0100707)

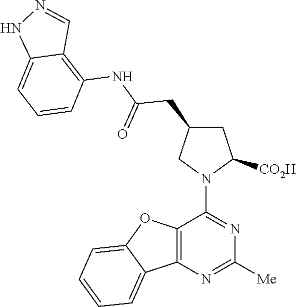 [SMILES: Cc1nc(N2C[C@@H](CC(=O)Nc3cccc4[nH]ncc34)C[C@H]2C(=O)O)c2oc3ccccc3c2n1]

¹HNMR (500 MHz, DMSO-d₆) δ 13.05 (br, 1H), 10.06 (s, 1H), 8.29 (s, 1H), 8.09 (d, J=8 Hz, 1H), 7.81 (br, 1H), 7.69 (m, 2H), 7.50 (t, 1H), 7.26 (m, 2H), 5.20 (br, 1H), 4.65 -4.36 (m, 1H), 3.74 (br, 1H), 2.9-2.4 (m, overlapped with DMSO, 7H), 1.85 (m, 1H); (ESI) m/z calcd for C₂₅H₂₂N₆O₄: 470.17. Found: 471.20 (M+1)⁺.

Compound 29: (2S,4R)-4-(2-((1H-benzo[d]imidazol-6-yl)amino)-2-oxoethyl)-1-(2-methylbenzofuro[3,2-d]pyrimidin-4-yl)pyrrolidine-2-carboxylic acid (BBL-0100714)

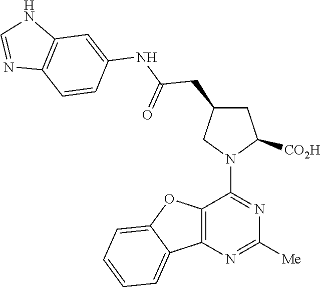 [SMILES: Cc1nc(N2C[C@@H](CC(=O)Nc3ccc4nc[nH]c4c3)C[C@H]2C(=O)O)c2oc3ccccc3c2n1]

¹HNMR (400 MHZ, DMSO-d₆) δ 12.72 (br, 1H), 10.14 (s, 1H), 8.17 (s, 1H), 8.07 (d, J=8 Hz, 1H), 7.96 (s, 1H), 7.77 (br, 1H), 7.66 (m, 2H), 7.48 (t, 1H), 7.11 (d. 1H), 5.15 (br. 1H), 4.70-4.40 (m, 1H), 3.72 (br, 1H), 2.9-2.4 (m, overlapped with DMSO, 7H), 1.85 (br d, 1H); (ESI) m/z calcd for C₂₅H₂₂N₆O₄: 470.17. Found: 471.10 (M+1)⁺.

Compound 30: (28.4R)-4-(2-((1H-indazol-6-yl)amino)-2-oxoethyl)-1-(2-methylbenzofuro[3,2-d]pyrimidin-4-yl)pyrrolidine-2-carboxylic acid (BBL-0100715)

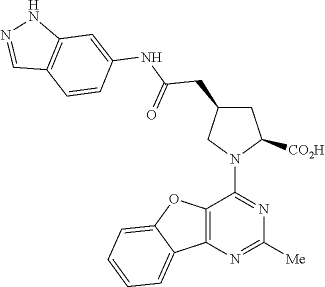 [SMILES: Cc1nc(N2C[C@@H](CC(=O)Nc3ccc4cn[nH]c4c3)C[C@H]2C(=O)O)c2oc3ccccc3c2n1]

¹HNMR (400 MHZ, DMSO-d₆) δ 12.50 (br, 1H), 10.00 (s. 1H), 8.14 (s. 1H), 8.07 (d, J=8 Hz, 2H), 7.78 (br, 1H), 7.66 (m, 1H), 7.50 (t and m, 2H), 7.24 (d, 1H), 5.13 (br, 1H), 4.58-4.30 (m, 1H), 3.72 (br, 1H), 2.9-2.4 (m, overlapped with DMSO, 7H), 1.85 (m, 1H) ; (ESI) m/z calcd for C₂₅H₂₂N₆O₄: 470.17. Found: 471.10 (M+1)⁺.

Compound 31: (2S,4R)-1-(2-methylbenzofuro[3,2-d]pyrimidin-4-yl)-4-(2-(naphthalen-2-ylamino)-2-oxoethyl)pyrrolidine-2-carboxylic acid (BBL-0100728)

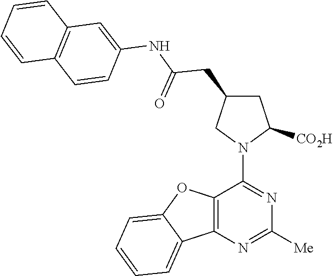 [SMILES: Cc1nc(N2C[C@@H](CC(=O)Nc3ccc4ccccc4c3)C[C@H]2C(=O)O)c2oc3ccccc3c2n1]

¹HNMR (400 MHZ, DMSO-d₆) § 10.22 (s, 1H), 8.34 (s, 1H), 8.04 (br, 1H), 7.90 -7.70 (m, 3.5H), 7.70-7.50 (m, 2.5H), 7.50-7.30 (m, 3H), 5.07 (br, 1H), 4.62-4.24 (m, 1H), 3.92-3.74 (m, 1H), 2.9-2.4 (m, overlapped with 7H), 2.0-1.7 (m, 1H); (ESI) m/z calcd for C₂₈H₂₄N₄O₄: 480.18. Found: 481.01 (M+1)⁺.

Compound 32: (2S,4R)-1-(2-methylbenzofuro[3,2-d]pyrimidin-4-yl)-4-(2-oxo-2-(quinolin-6-ylamino)ethyl)pyrrolidine-2-carboxylic acid (BBL-0100729)

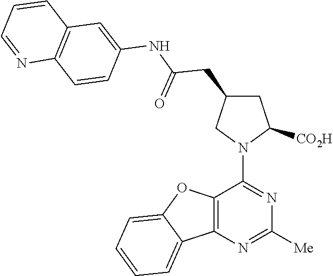 [SMILES: Cc1nc(N2C[C@@H](CC(=O)Nc3ccc4ncccc4c3)C[C@H]2C(=O)O)c2oc3ccccc3c2n1]

¹HNMR (400 MHz, DMSO-d₆) δ 12.80 (br, 1H), 10.35 (s, 1H), 8.78 (m, J=5 Hz, 1H), 8.43 (d. J=˜2 Hz, 1H), 8.29 (d. J=10 Hz, 1H), 8.07 (d, J=10 Hz, 1H), 7.95 (d, J=10 Hz. 1H), 7.80 (m, 2H), 7.66 (m, 1.5H), 7.47 (t, J=10 Hz, 2H), 5.13 (br, 1H), 4.60-4.30 (m, 1H), 3.75 (br, 1H), 2.9-2.4 (m, overlapped with DMSO. 7H), 1.80 (m. 1H): (ESI) m/z calcd for C₂₇H₂₃N₅O₄: 481.18. Found: 482.01 (M+1)⁺.

Compound 33: (2S,4R)-4-(2-(isoquinolin-6-ylamino)-2-oxoethyl)-1-(2-methylbenzofuro[3,2-d]pyrimidin-4-yl)pyrrolidine-2-carboxylic acid (BBL-0100708)

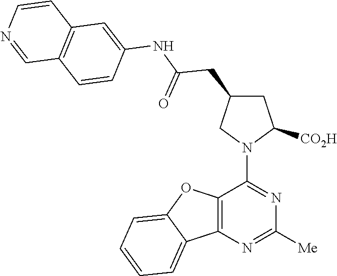 [SMILES: Cc1nc(N2C[C@@H](CC(=O)Nc3ccc4cnccc4c3)C[C@H]2C(=O)O)c2oc3ccccc3c2n1]

¹HNMR (400 MHZ, CD₃OD) 8 9.54 (s, 1H), 8.82 (br s, 1H), 8.45 (m, 2H), 8.30 (d. 1H), 8.15 (d, J=8 Hz, 1H), 8.04 (m, 1H), 7.75-7.75 (m. 3H), 7.62 (m, 1H), 5.50 (br, 1H), 4.97-4.67 (m, 1H), 4.05-3,66 (m, 1H), 3.1-2.7 (m, 4H), 2.75 (s. 3H), 2.1-1.9 (m, 1H); (ESI) m/z calcd for C₂₇H₂₃N₅O₄: 481.18. Found: 482.31 (M+1)⁺.

Compound 34: (28.4R)-4-(2-(isoquinolin-7-ylamino)-2-oxoethyl)-1-(2-methylbenzofuro[3,2-d]pyrimidin-4-yl)pyrrolidine-2-carboxylic acid (BBL-0100722)

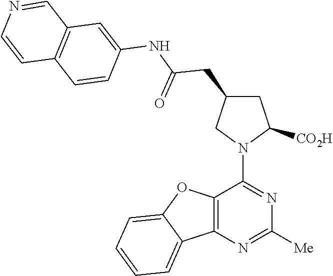 [SMILES: Cc1nc(N2C[C@@H](CC(=O)Nc3ccc4ccncc4c3)C[C@H]2C(=O)O)c2oc3ccccc3c2n1]

¹HNMR (500 MHZ, CD₃OD) 6 9.14 (br, 1H), 8.58 (br, 1H), 8.34 (br. 1H), 8.09 (br, 1H), 7.82 (m, 2H), 7.88 (m, 1H), 7.66 (m, 2H), 7.46 (m, 1H), 5.36 (br, 1H), 4.9-4.61 (m, 1H), 4.26-4.07 (m, 1H), 3.75 (br, overlapped, 1H), 3.1-2.5 (m, overlapped, 6H), 2.45 (m, 1H); (ESI) m/z calcd for C₂₇H₂₃N₅O₄: 481.18. Found: 482.21 (M+1)⁺.

Compound 35: (28,4R)-1-(2-methylbenzofuro[3,2-d]pyrimidin-4-yl)-4-(2-oxo-2-(quinolin-7-ylamino)ethyl)pyrrolidine-2-carboxylic acid (BBL-0100723)

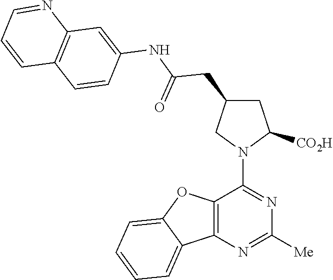 [SMILES: Cc1nc(N2C[C@@H](CC(=O)Nc3ccc4cccnc4c3)C[C@H]2C(=O)O)c2oc3ccccc3c2n1]

¹HNMR (500 MHz, CD₃OD) 5 9.14 (br, 1H), 8.48 (br. 1H), 8.31 (br. 1H). 8.12 (br, 1H), 7.88 (m, 1H), 7.77 (m, 1H), 7.66 (m, 2H), 7.46 (m, 1H), 5.21 (br, 1H), 4.9-4.4 (m, 1H), 3.89-3.57 (m. 1H), 3.0 -2.5 (m, overlapped, 7H), 2.00 (m, 1H); (ESI) m/z calcd for C₂₇H₂₃N₅O₄: 481.18. Found: 482.22 (M+1)⁺.

Compound 36: (2S,4R)-4-(2-([1,1′-biphenyl]-3-ylamino)-2-oxoethyl)-1-(2-methylbenzofuro[3,2-d]pyrimidin-4-yl)pyrrolidine-2-carboxylic acid (BBL-0100727)

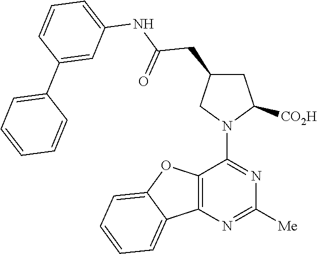 [SMILES: Cc1nc(N2C[C@@H](CC(=O)Nc3cccc(-c4ccccc4)c3)C[C@H]2C(=O)O)c2oc3ccccc3c2n1]

¹HNMR (400 MHZ, DMSO-d₆) δ 12.63 (br, 1H). 10.11 (s, 1H), 8.06 (d. J=4 Hz, 1H). 7.95 (m, 1H), 7.76 (br, 1H), 7.70-7.55 (m, 4H), 7.50-7.42 (m, 3H), 7.40-7.30 (m, 3H), 5.3 (br, 1H), 4.60-4.28 (m, 1H), 3.73 (br. 1H), 2.9-2.4 (m, overlapped with DMSO, 7H). 1.80 (br d, 1H); (ESI) m/z calcd for C₃₀H₂₅N₄O₄: 506.20. Found: 507.01 (M+1)⁺.

Compound 37: (2S,4R)-1-(2-methylbenzofuro[3,2-d]pyrimidin-4-yl)-4-(2-oxo-2-((4-(pyridin-3-yl)phenyl)amino)ethyl)pyrrolidine-2-carboxylic acid (BBL-0100735)

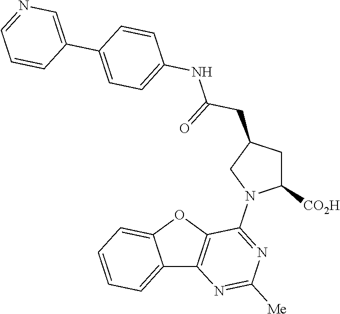 [SMILES: Cc1nc(N2C[C@@H](CC(=O)Nc3ccc(-c4cccnc4)cc3)C[C@H]2C(=O)O)c2oc3ccccc3c2n1]

¹HNMR (500 MHz, DMSO-d₆) δ 12.85(br, 1H), 10.16 (s. 1H), 8.87 (d, J=1 Hz, 1H), 8.53 (d. J=5, 1 Hz, 1H), 8.06 (m, 2H), 7.79 (br 1H), 7.68 (A and B of ABq, J=10 Hz, 2H+2H), 7.65 (m, 1H), 7.46 (m, 2H), 5.11 (br, 1H), 4.60-4.26 (m, 1H), 3.73 (br s, 1H), 2.9-2.4 (m, overlapped with DMSO, 7H), 1.90-1.74 (m, 1H): (ESI) m/z calcd for C₂₉H₂₃N₅O₄: 507.19. Found: 508.51 (M+1)⁺.

Compound 38: (2S,4R)-4-(2-((4-(1H-indazol-5-yl)phenyl)amino)-2-oxoethyl)-1-(2-methylbenzofuro[3,2-d]pyrimidin-4-yl)pyrrolidine-2-carboxylic acid (BBL-0100743)

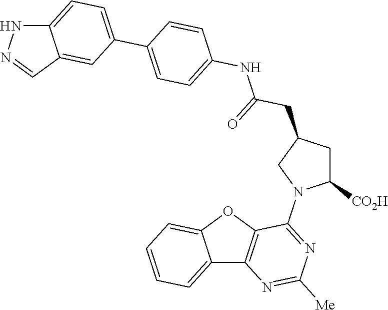 [SMILES: Cc1nc(N2C[C@@H](CC(=O)Nc3ccc(-c4ccc5[nH]ncc5c4)cc3)C[C@H]2C(=O)O)c2oc3ccccc3c2n1]

¹HNMR (500 MHz, DMSO-d₆) δ 8.12 (d, 1H, NH), 8.08 (d, 1H), 7.97 (s. 1H), 7.62˜7.75 (m, 6H), 7.60 (m, 2H), 7.46 (m, 2H), 5.23 (m, 1H), 4.58-4.44 (m, 1H), 4.04 -3.89 (m. 1H), 3.58 (m, 1H), 3.0-2.5 (m, overlapped with DMSO, 6H), 2.06-1.90 (m, 1H); (ESI) m/z calcd for C₃₁H₂₆N₆O₄: 546.20. Found: 547.51 (M+1)⁺.

Compound 39: (28.4R)-4-(2-((4-(1H-indazol-6-yl)phenyl)amino)-2-oxoethyl)-1-(2-methylbenzofuro[3,2-d]pyrimidin-4-yl)pyrrolidine-2-carboxylic acid (BBL-0100741)

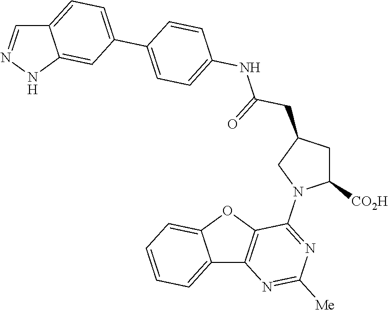 [SMILES: Cc1nc(N2C[C@@H](CC(=O)Nc3ccc(-c4ccc5cn[nH]c5c4)cc3)C[C@H]2C(=O)O)c2oc3ccccc3c2n1]

¹HNMR (500 MHz, DMSO-d₆) δ 13.08 (m, 1H), 10.28 (s, 1H), 8.07 (m, 2H), 7.80 (d, J=5 Hz, 2H), 7.73 (m. 1H), 7.69 (m, 3H), 7.62 (br, 1H), 7.45 (m, 1H), 7.40 (m, 1H), 5.19 (br s, 1H), 4.63-4.26 (m, 1H), 3.87-3.74 (m, 1H), 2.9-2.4 (m, overlapped with DMSO, 7H). 1.91-1.75(m, 1H) ; (ESI) m/z calcd for C₃₁H₂₆N₆O₄: 546.20. Found: 547.52 (M+1)⁺.

Compound 40: (2S,4R)-4-(2-((4-(6-aminopyridin-3-yl)phenyl)amino)-2-oxoethyl)-1-(2-methylbenzofuro[3,2-d]pyrimidin-4-yl)pyrrolidine-2-carboxylic acid (BBL-0150979)

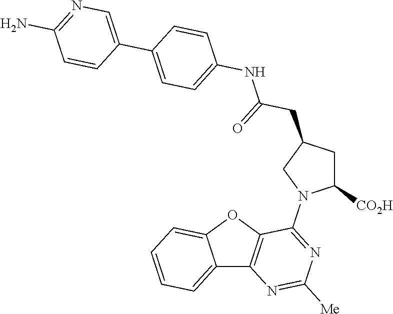 [SMILES: Cc1nc(N2C[C@@H](CC(=O)Nc3ccc(-c4ccc(N)nc4)cc3)C[C@H]2C(=O)O)c2oc3ccccc3c2n1]

¹HNMR (500 MHz, CD₃OD) 5 8.24 (s, 1H), 8.11 (br, 2H), 7.90 (d, 1H), 7.68 (B of ABq. J=8 Hz, 2H), 7.62 (m, 2H), 7.51 (A of ABq, J=8 Hz, 2H). 7.44 (m, 1H), 6.80 (br d. J=10 Hz, 1H), 5.18 (br s, 1H), 4.76-4.36 (m, 1H), 3.95-3.60 (m, 1H), 3.0-2.5 (m, 7H), 2.07-1.89 (m, 1H) ; (ESI) m/z calcd for C₂₉H₂₆N₆O₄: 522.20. Found: 523.52 (M+1)⁺.

Compound 41: (28.4R)-4-(2-((4-(2-aminopyridin-4-yl)phenyl)amino)-2-oxoethyl)-1-(2-methylbenzofuro[3,2-d]pyrimidin-4-yl)pyrrolidine-2-carboxylic acid (BBL-0150980)

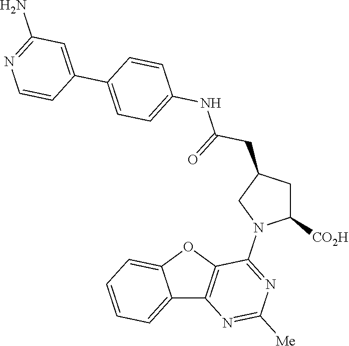 [SMILES: Cc1nc(N2C[C@@H](CC(=O)Nc3ccc(-c4ccnc(N)c4)cc3)C[C@H]2C(=O)O)c2oc3ccccc3c2n1]

¹HNMR (500 MHz, CD₃OD) δ 8.27 (s, 1H), 8.12 (br, 1H), 7.86 (d, 1H), 7.77 (B of ABq, 2H). 7.71 (A of ABq, 2H), 7.64 (m, 2H), 7.43 (m, 1H), 7.07 (br d, J=10 Hz, 1H), 7.02 (s, 1H), 5.16 (br s, 1H), 4.76-4.35 (m, 1H), 3.94-3.60 (m, 1H), 3.0-2.5 (m, 7H), 2.16 -1.89 (m, 1H); (ESI) m/z calcd for C₂₉H₂₆N₆O₄: 522.20. Found: 523.42 (M+1)⁺.

Compound 42: (2S,4R)-4-(2-((4-(6-aminopyridin-2-yl)phenyl)amino)-2-oxoethyl)-1-(2-methylbenzofuro[3.2-d]pyrimidin-4-yl)pyrrolidine-2-carboxylic acid (BBL-0150981)

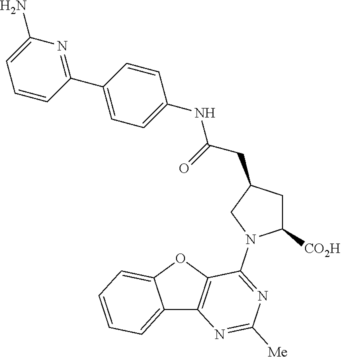 [SMILES: Cc1nc(N2C[C@@H](CC(=O)Nc3ccc(-c4cccc(N)n4)cc3)C[C@H]2C(=O)O)c2oc3ccccc3c2n1]

¹HNMR (500 MHz, DMSO-d₆) δ 10.10 (s, 1H), 8.14 (s, 1H), 8.06 (d, 1H), 7.93 (d, J=10 Hz, 2H), 7.77 (br, 1H), 7.68 (d. J=10 Hz, 2H). 7.65 (br t, 1H), 7.43 (m, 2H), 7.01 (d, J=10 Hz, 1H), 6.38 (d. J=10 Hz, 1H), 5.91 (s, 2H), 5.11 (br s, 1H), 4.61-4.27 (m, 1H), 3.72 (m, 1H), 2.9-2.4 (m, overlapped with DMSO, 7H), 1.90-1.73 (m, 1H) : (ESI) m/z calcd for C₂₉H₂₆N₆O₄: 522.20. Found: 523.42 (M+1)⁺.

Compound 43: (28.4R)-4-(2-((4-(2-aminopyridin-3-yl)phenyl)amino)-2-oxoethyl)-1-(2-methylbenzofuro[3,2-d]pyrimidin-4-yl)pyrrolidine-2-carboxylic acid (BBL-0150982)

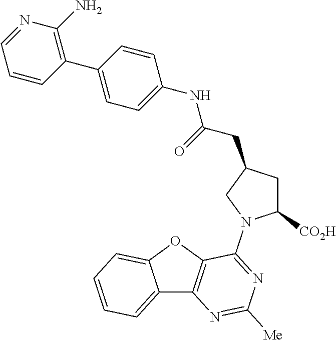 [SMILES: Cc1nc(N2C[C@@H](CC(=O)Nc3ccc(-c4cccnc4N)cc3)C[C@H]2C(=O)O)c2oc3ccccc3c2n1]

¹HNMR (500 MHZ, CD₃OD) δ 8.12 (br, 1H), 7.89 (m, 1H), 7.74 (d. 2H), 7.64 (m, 2H), 7.56 (d, 1H), 7.46 (m, 3H), 6.82 (dd, J=10 Hz, 1H), 5.19 (br s, 1H), 4.76-4.36 (m, 1H), 3.92-3.58 (m, 1H), 3.0-2.5 (m, 7H), 1.99-1.89 (m, 1H); (ESI) m/z calcd for C₂₉H₂₆N₆O₄: 522.20. Found: 523.52 (M+1)⁺.

Compound 44: (2S,4R)-1-(2-methylbenzofuro[3,2-d]pyrimidin-4-yl)-4-(2-oxo-2-((4-(pyridazin-3-yl)phenyl)amino)ethyl)pyrrolidine-2-carboxylic acid (BBL-0150972)

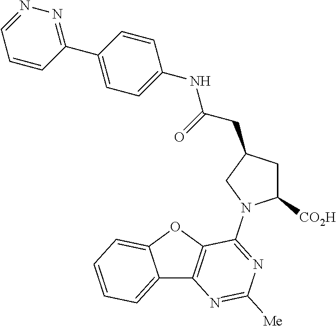 [SMILES: Cc1nc(N2C[C@@H](CC(=O)Nc3ccc(-c4cccnn4)cc3)C[C@H]2C(=O)O)c2oc3ccccc3c2n1]

¹HNMR (500 MHz, DMSO-d₆) δ 12.63 (br, 1H), 10.25 (s, 1H), 9.16 (dd, 1H), 8.18 (dd, J=10 Hz, 1H), 8.13 (B of ABq, 2H), 8.05 (d, 1H), 7.80 (A of ABq, J=10 Hz, 2H), 7.74 (m, 1H), 7.65 (t, 1 H), 7.46 (t, 1H), 5.15 (br s, 1H), 4.62-4.42 (m, 1H), 3.7 (br, 1H), 2.9-2.4 (m, overlapped with DMSO, 7H), 1.90-1.75 (m, 1H); (ESI) m/z calcd for C₂₉H₂₆N₆O₄: 508.20. Found: 509.42 (M+1)⁺.

Compound 45: (28.4R)-1-(2-methylbenzofuro[3,2-d]pyrimidin-4-yl)-4-(2-oxo-2-((4-(pyridazin-4-yl)phenyl)amino)ethyl)pyrrolidine-2-carboxylic acid (BBL-0150991)

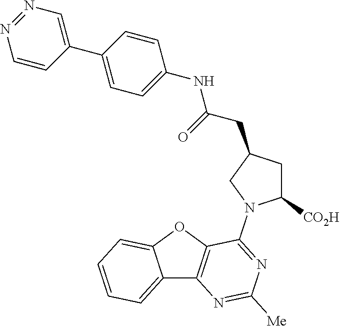 [SMILES: Cc1nc(N2C[C@@H](CC(=O)Nc3ccc(-c4ccnnc4)cc3)C[C@H]2C(=O)O)c2oc3ccccc3c2n1]

¹HNMR (400 MHZ, CD₃OD) δ 9.44 (m, J=<1 Hz, 1H), 9.06 (d, J=4 Hz, 1H), 8.02 (m, 1H), 7.89 (m, 1H), 7.72 (m, 4H), 7.55 (m, 2H), 7.36 (m, 1H), 5.13 (br s, 1H), 4.75-4.34 (m, 1H), 378-3.48 (m, 1H), 3.01-2.52 (m, 7H), 2.00-1.79 (m, 1H) ; (ESI) m/z calcd for C₂₈H₂₄N₆O₄: 508.20. Found: 509.32 (M+1)⁺.

Compound 46: (2S,4R)-4-(2-((4-(1H-indazol-4-yl)phenyl)amino)-2-oxoethyl)-1-(2-methylbenzofuro[3,2-d]pyrimidin-4-yl)pyrrolidine-2-carboxylic acid (BBL-0100742)

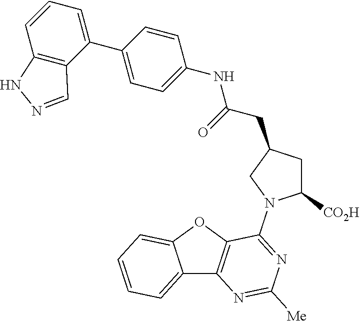 [SMILES: Cc1nc(N2C[C@@H](CC(=O)Nc3ccc(-c4cccc5[nH]ncc45)cc3)C[C@H]2C(=O)O)c2oc3ccccc3c2n1]

¹HNMR (500 MHz, DMSO-d₆) δ 8.17 (d, 1H), 8.13 (d, 1H), 7.77 (d, 1H), 7.69 (m, 2H), 7.65 (m, 2H), 7.51 (d. J=10 Hz, 1H), 7.46 (m, 2H). 7.24 (d, J=10 Hz, 1H), 5.22 (m, 1H). 4.63-4.43 (m, 1H), 3.91-3.58 (m, 1H), 2.91-2.52 (m, overlapped with DMSO, 7H), 2.06 - 2.90 (m, 1H); (ESI) m/z calcd for C₃₁H₂₆N₆O₄: 546.20. Found: 547.52 (M+1)⁺.

Compound 47: (28.4R)-4-(2-((4-(6-hydroxypyridin-3-yl)phenyl)amino)-2-oxoethyl)-1-(2-methylbenzofuro[3,2-d]pyrimidin-4-yl)pyrrolidine-2-carboxylic acid (BBL-0150984)

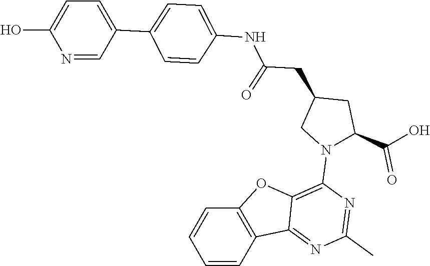 [SMILES: Cc1nc(N2C[C@@H](CC(=O)Nc3ccc(-c4ccc(O)nc4)cc3)C[C@H]2C(=O)O)c2oc3ccccc3c2n1]

¹HNMR (500 MHz, CD₃OD) δ 8.11 (br d, 1H), 7.94 (dd, J=5, 10 Hz, 1H), 7.66 (m, 5H), 7.45˜7.55(m, 3H), 6,64 (d, J=5 Hz, 1H), 5.23 (br s, 1H), 4.74-4.43 (m, 1H), 3.88-3.55 (m, 1H), 3.29-2.51 (m, 7H), 1.88 (m, 1H); (ESI) m/z calcd for C₂₉H₂₅N₅O₅: 523.19. Found: 524.37 (M+1)⁺.

Compound 48: (2S,4R)-4-(2-((4-(2-hydroxypyridin-4-yl)phenyl)amino)-2-oxoethyl)-1-(2-methylbenzofuro[3,2-d]pyrimidin-4-yl)pyrrolidine-2-carboxylic acid (BBL-0150985)

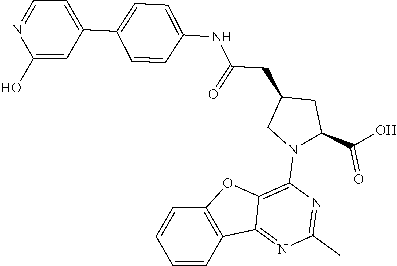 [SMILES: Cc1nc(N2C[C@@H](CC(=O)Nc3ccc(-c4ccnc(O)c4)cc3)C[C@H]2C(=O)O)c2oc3ccccc3c2n1]

¹H NMR (400 MHZ, DMSO-d₆) δ 11.56 (br, 1H), 10.22 (s, 1H), 8.04 (s, 1H), 7.77 (m, 1H), 7.72 (d, J=8.7 Hz, 2H), 7.68-7.56 (m, 4H), 7.50-7.36 (m, 2H), 6.55 (s, 1H), 6.52-6.44 (m, 1H), 5.20-4.58 (m, 1H), 4.57-4.16 (m, 1H), 3.79-3.65 (m, 1H), 2.88-2.51 (m, 7H), 1.98-1.66 (m, 1H); (ESI) m/z calcd for C₂₉H₂₆N₅O₅: 523.19. Found: 524.37 (M+1)⁺.

Compound 49: (28,4R)-4-(2-((3-(1H-imidazol-4-yl)phenyl)amino)-2-oxoethyl)-1-(2-methylbenzofuro[3,2-d]pyrimidin-4-yl)pyrrolidine-2-carboxylic acid (BBL-0100747)

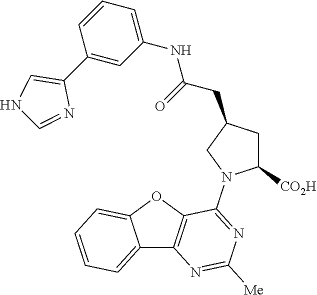 [SMILES: Cc1nc(N2C[C@@H](CC(=O)Nc3cccc(-c4c[nH]cn4)c3)C[C@H]2C(=O)O)c2oc3ccccc3c2n1]

¹HNMR (500 MHZ, DMSO-d₆) δ 12.16 (br, 1H), 10.02 (s, 1H), 8.02 (br, 2H), 7.75 (m, 1H), 7.68 (s, 1H), 7.58 (br, 2H), 7.3˜7.5 (m, 3H), 7.25 (br, 1H), 4.98 (br s, 1H), 4.60-4.15 (m 1H), 3.76 (m, 1H), 3.01-2.53 (m, overlapped with DMSO, 7H), 1.92-1.79 (m, 1H) : (ESI) m/z caled for C₂₇H₂₄N₆O₄: 496.19. Found: 497.4 (M+H)⁺.

Compound 50: (2S,4R)-4-(2-((3′-methoxy-[1,1′-biphenyl]-4-yl)amino)-2-oxoethyl)-1-(2-methylbenzofuro[3,2-d]pyrimidin-4-yl)pyrrolidine-2-carboxylic acid (BBL-0150971)

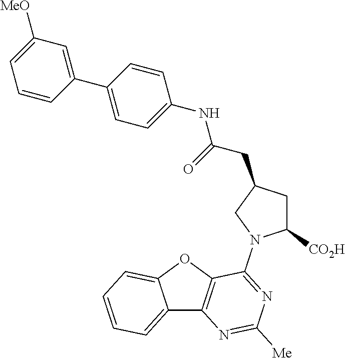 [SMILES: COc1cccc(-c2ccc(NC(=O)C[C@H]3C[C@@H](C(=O)O)N(c4nc(C)nc5c4oc4ccccc45)C3)cc2)c1]

¹HNMR (500 MHZ, DMSO-d₆) δ 10.11 (d, 1H), 8.10 (br, 1H), 7.78 (br, 1H), 7.68 (m, 1H), 7.62 (m, 3H), 7.44 (br, 1H), 7.20 (d, J=5 Hz, 1H), 7.15 (s, 1H), 6.88 (m, 1H), 5.16 (br s, 1H), 4.74-4.22 (m, 1H), 3.91 (s, 3H), 3.73 (br s, 1H), 2.91-2.50 (m, overlapped with DMSO, 7H), 1.90-1.75 (m. 1H); (ESI) m/z calcd for C₃₁H₂₈N₄O₅: 536.21. Found: 537.4 (M+H)⁺.

Compound 51: (2S,4R)-4-(2-((3′-hydroxy-[1,1′-biphenyl]-4-yl)amino)-2-oxoethyl)-1-(2-methylbenzofuro[3,2-d]pyrimidin-4-yl)pyrrolidine-2-carboxylic acid (BBL-0150983)

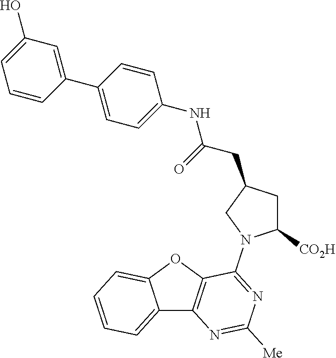 [SMILES: Cc1nc(N2C[C@@H](CC(=O)Nc3ccc(-c4cccc(O)c4)cc3)C[C@H]2C(=O)O)c2oc3ccccc3c2n1]

¹HNMR (500 MHZ, CD₃OD) δ 8.13 (d, 1H), 7.65 (m, 4H), 7.56 (d, J=10 Hz, 2H), 7.46 (m, 1H), 7.23 (m, 1H), 7.07 (d, J=10 Hz, 1H), 7.02 (m, 1H), 6.75 (dd, J=10 Hz, 1H), 5.25 (br s, 1H), 4.86-4.35 (m, 1H), 3.90 (m, 1H), 2.90-2.51 (m, 7H), 2.04-1.89 (m, 1H); (ESI) m/z calcd for C₃₀H₂₆N₄O₅: 522.19. Found: 523.38 (M+H)⁺

Compound 52: (2S,4R)-1-(2-methylbenzofuro[3,2-d]pyrimidin-4-yl)-4-(2-oxo-2-((4-(2-oxooxazolidin-3-yl)phenyl)amino)ethyl)pyrrolidine-2-carboxylic acid (BBL-0150986)

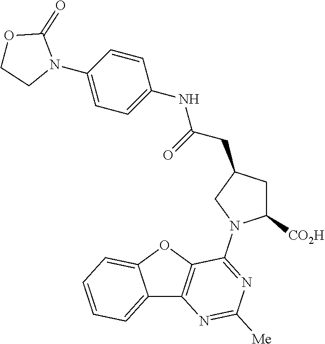 [SMILES: Cc1nc(N2C[C@@H](CC(=O)Nc3ccc(N4CCOC4=O)cc3)C[C@H]2C(=O)O)c2oc3ccccc3c2n1]

¹HNMR (500 MHz, DMSO-d₆) δ 10.00 (br, 1H), 8.06 (d, 1H), 7.79 (br, 1H), 7.6˜7.7 (m, 3H), 7.4˜7.5 (m, 3H), 5.15 (br s. 1H), 4.61-4.27 (m, 1H), 4.42 (dd, 2H), 4.03 (dd, 2H), 3.72 (m, 1H), 2.90-2.51 (m, overlapped with DMSO, 7H), 1.90-1.75 (m, 1H); (ESI) m/z calcd for C₂₇H₂₅N₅O₆: 515.18. Found: 516.32 (M+H)⁺

Compound 53: (2S,4R)-4-(2-((4-(4-hydroxypiperidin-1-yl)phenyl)amino)-2-oxoethyl)-1-(2-methylbenzofuro[3,2-d]pyrimidin-4-yl)pyrrolidine-2-carboxylic acid (BBL-0100748)

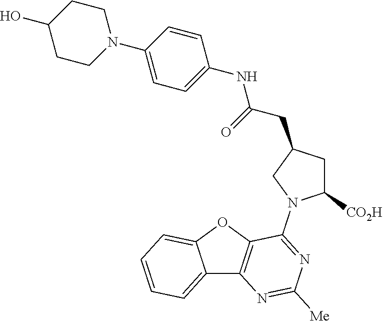 [SMILES: Cc1nc(N2C[C@@H](CC(=O)Nc3ccc(N4CCC(O)CC4)cc3)C[C@H]2C(=O)O)c2oc3ccccc3c2n1]

¹HNMR (500 MHz, DMSO-d₆) δ 8.13 (d, 1H), 7.86 (br, 1H), 7.75 (br, 1H), 7.5˜7.65 (m. 2H), 6.98, 7.08, 7.18 (m, 3H). 5.23 (br s, 1H), 4.68-4.35 (m, 1H), 3.53 (m, 10H), 3.01-2.52 (m, overlapped with DMSO, 7H), 1.92-1.61 (m, 1H) ; (ESI) m/z calcd for C₂₉H₃₁N₅O₅: 529.23. Found: 530.50 (M+H)⁺

Compound 54: (28.4R)-4-(2-((4-benzylphenyl)amino)-2-oxoethyl)-1-(2-methylbenzofuro[3,2-d]pyrimidin-4-yl)pyrrolidine-2-carboxylic acid (BBL-0100745)

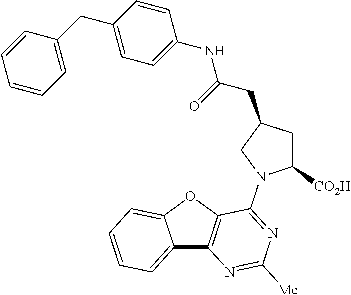

¹HNMR (500 MHz, DMSO-d₆) δ 9.94 (s, 1H), 8.03 (br, 1H), 7.77 (br, 1H), 7.59 (br. 1H), 7.51 (d, 1H), 7.42 (br, 1H), 7.27 (m, 2H), 7.1˜7.21 (m,5H), 5.01 (br s, 1H), 4.59-4.16 (m, 1H), 3.87 (s, 2H), 3.69 (br s, 1H), 3.01-2.52 (m, overlapped with DMSO, 7H), 1.89-1.70 (m, 1H) : (ESI) m/z calcd for C₃₁H₂₈N₄O₄: 520.21. Found: 521.55 (M+H)⁺

Compound 55: (2S,4R)-1-(2-methylbenzofuro[3,2-d]pyrimidin-4-yl)-4-(2-oxo-2-((4-(pyridin-3-ylmethyl)phenyl)amino)ethyl)pyrrolidine-2-carboxylic acid (BBL-0150992)

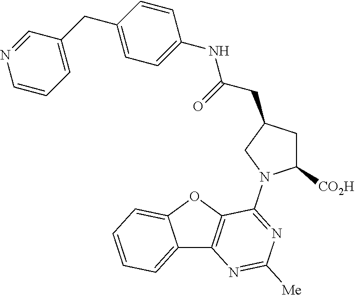 [SMILES: Cc1nc(N2C[C@@H](CC(=O)Nc3ccc(Cc4cccnc4)cc3)C[C@H]2C(=O)O)c2oc3ccccc3c2n1]

¹HNMR (500 MHz, CD₃OD) 5 8.43 (br s. 1H), 8.37 (br, 1H), 8.12 (d, 1H), 7.69 (m, 1H), 7.66 (d, 2H), 7.53 (d, J=10 Hz, 2H), 7.46 (m, 1H), 7.37 (m, 1H), 7.19 (d, J=10 Hz, 2H), 5.21(br s. 1H), 4.74-4.41 (m, 1H), 4.00 (s, 2H), 3.86-3.52 (m, 1H), 3.01-2.52 (m. overlapped with DMSO, 7H), 2.05-1.86 (m, 1H); (ESI) m/z calcd for C₃₀H₂₇N₅O₄: 521.21. Found: 522.37 (M+H)⁺

Compound 56: (2S,4R)-1-(2-methylbenzofuro[3,2-d]pyrimidin-4-yl)-4-(2-oxo-2-((4-(pyridin-2-ylmethyl)phenyl)amino)ethyl)pyrrolidine-2-carboxylic acid (BBL-0150975)

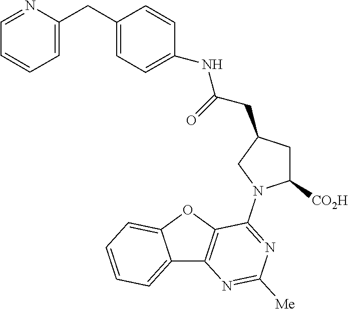 [SMILES: Cc1nc(N2C[C@@H](CC(=O)Nc3ccc(Cc4ccccn4)cc3)C[C@H]2C(=O)O)c2oc3ccccc3c2n1]

¹HNMR (500 MHz, DMSO-d₆) δ 9.94 (s, 1H), 8.47 (d, J=5 Hz, 1H), 8.06 (br, 1H), 7.77 (br, 1H), 7.70 (td, 1H), 7.62 (br, 1H), 7.51 (B of ABq, J=10 Hz, 2H), 7.45 (br, 1H), 7.24 (d. J=10 Hz, 2H). 7.19 (A of ABq, J=10 Hz, 2H), 5.10 (br s, 1H), 4.60-4.24 (m, 1H), 4.01 (s, 2H), 3.69 (m, 1H), 3.01-2.52 (m, overlapped with DMSO, 7H), 1.90-1.70 (m, 1H); (ESI) m/z calcd for C₃₀H₂₇N₅O₄: 521.21. Found: 522.50 (M+H)⁺

Compound 57: (2S,4R)-1-(2-methylbenzofuro[3,2-d]pyrimidin-4-yl)-4-(2-((3-morpholinophenyl)amino)-2-oxoethyl)pyrrolidine-2-carboxylic acid (BBL-0150974)

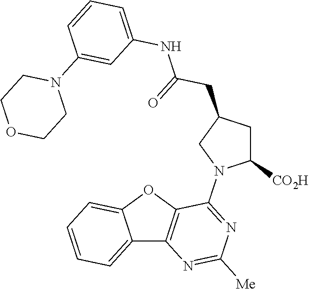 [SMILES: Cc1nc(N2C[C@@H](CC(=O)Nc3cccc(N4CCOCC4)c3)C[C@H]2C(=O)O)c2oc3ccccc3c2n1]

¹HNMR (500 MHz, DMSO-d₆) δ 8.05 (br, 1H), 9.87 (s, 1H), 7.80 (br, 1H), 7.65 (br, 1H), 7.45 (br, 1H), 7.29 (s, 1H), 7.13 (t, 1H), 7.02 (d, 1H), 6.61 (d, 1H), 5.10 (br s, 1H), 4.60-4.24 (m. 1H), 3.70 (m, 1H), 3.74 (t, 4H), 3.06 (t, 4H), 2.81-2.42 (m, overlapped with DMSO, 7H), 1.87-1.71 (m. 1H) ; (ESI) m/z calcd for C₂₈H₂₉N₅O₅: 515.22. Found: 516.51 (M+H)⁺

Compound 58: (2S,4R)-4-(2-((3-(4-hydroxypiperidin-1-yl)phenyl)amino)-2-oxoethyl)-1-(2-methylbenzofuro[3,2-d]pyrimidin-4-yl)pyrrolidine-2-carboxylic acid (BBL-0150973)

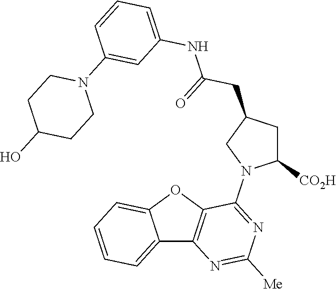 [SMILES: Cc1nc(N2C[C@@H](CC(=O)Nc3cccc(N4CCC(O)CC4)c3)C[C@H]2C(=O)O)c2oc3ccccc3c2n1]

¹HNMR (500 MHz, DMSO-d₆) δ 9.87 (s, 1H), 8.06 (br. 1H), 7.78 (br, 1H), 7.63 (br, 1H), 7.45 (br, 1H), 7.26 (s, 1H), 7.10 (t, 1H), 6.97 (m, 1H), 6.61 (d, 1H), 5.10 (br s, 1H), 4.60 - 4.23 (m, 1H), 3.91-3.31 (m, 4H), 2.82 (d of t, 1H), 2.81-2.42 (m, overlapped with DMSO, 8H), 1.90-1.60 (m, 4H), 1.45 (m, 1H) ; (ESI) m/z calcd for C₂₉H₃₁N₅O₅: 529.23. Found: 530.50 (M+H)⁺

Compound 59: (2S,4R)-4-(2-((3′-(hydroxymethyl)-[1,1′-biphenyl]-4-yl)amino)-2-oxoethyl)-1-(2-methylbenzofuro[3,2-d]pyrimidin-4-yl)pyrrolidine-2-carboxylic acid (BBL-0150987)

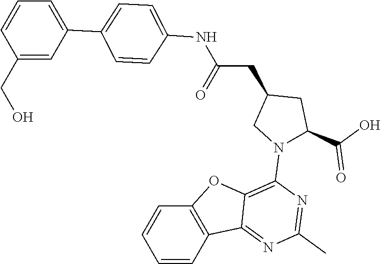 [SMILES: Cc1nc(N2C[C@@H](CC(=O)Nc3ccc(-c4cccc(CO)c4)cc3)C[C@H]2C(=O)O)c2oc3ccccc3c2n1]

¹HNMR (400 MHZ, DMSO-d₆) § 12.80 (br, 1H), 10.21 (s, 1H), 8.06 (m. 1H), 7.79 (br. 1H), 7.55˜7.75 (m, 6H), 7.49 (m. 1H), 7.45 (br s, 1H), 7.38 (m, 1H), 7.26 (d, J=8 Hz, 1H), 5.23 (m, 1H), 5.12-4.62 (m, 1H), 4.55 (s, 2H), 4.26-3.72 (m, 1H), 2.70-2.21 (m, overlapped with DMSO, 7H), 1.89-1.73 (m, 1H) ; (ESI) m/z calcd for C₃₁H₂₈N₄O₅: 536.21. Found: 537.36 (M+H)⁺.

Compound 60: (28.4R)-4-(2-((4′-(hydroxymethyl)-[1,1′-biphenyl]-4-yl)amino)-2-oxoethyl)-1-(2-methylbenzofuro[3,2-d]pyrimidin-4-yl)pyrrolidine-2-carboxylic acid (BBL-0150988)

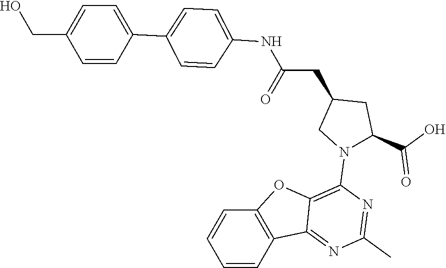 [SMILES: Cc1nc(N2C[C@@H](CC(=O)Nc3ccc(-c4ccc(CO)cc4)cc3)C[C@H]2C(=O)O)c2oc3ccccc3c2n1]

¹HNMR (400 MHZ, DMSO-d₆) δ 12.68 (br, 1H), 10.11 (s, 1H), 8.06 (br, 1H), 7.79 (br, 1H), 7.71 (d, J=8 Hz, 2H), 7.55˜7.65 (m, 5H), 7.46 (m, 1H), 7.38 (d, J=8 Hz, 2H), 5.15 (m, 1H), 4.61-4.56 (m, 1H), 4.51 (d, 2H), 4.27-3.72 (m, 1H), 2.71-2.35 (m, overlapped with DMSO, 7H), 1.89-1.73 (m, 1H); (ESI) m/z calcd for C₃₁H₂₈N₄O₅: 536.21. Found: 537.37 (M+H)⁺.

Compound 61: (2S,4R)-4-(2-((2-methoxy-[1,1′-biphenyl]-4-yl)amino)-2-oxoethyl)-1-(2-methylbenzofuro[3,2-d]pyrimidin-4-yl)pyrrolidine-2-carboxylic acid (BBL-0150993)

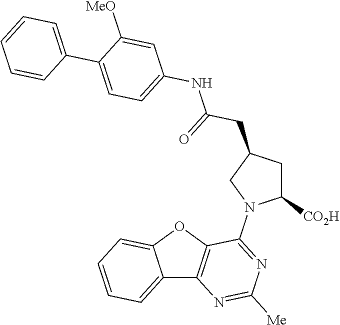 [SMILES: COc1cc(NC(=O)C[C@H]2C[C@@H](C(=O)O)N(c3nc(C)nc4c3oc3ccccc34)C2)ccc1-c1ccccc1]

¹HNMR (400 MHZ, CD₃OD) δ 8.11 (br d, 1H), 7.66 (br d, 2H), 7.42 ˜7.55 (m, 4H), 7.35 (m, 2H), 7.25 (m, 2H), 7.18 (dd, J=8 Hz, 1H), 5.24 (m, 1H), 4.74-4.44 (m, 1H), 3.90 (m. 1H), 3.80 (s, 3H), 3.56 (m, 1H), 3.01-2.52 (m, 6H), 1.89 (m, 1H); (ESI) m/z calcd for C₃₁H₂₈N₄O₅: 536.21. Found: 537.30 (M+H)⁺.

Compound 62: (2S,4R)-4-(2-((2-amino-[1,1′-biphenyl]-4-yl)amino)-2-oxoethyl)-1-(2-methylbenzofuro[3,2-d]pyrimidin-4-yl)pyrrolidine-2-carboxylic acid (BBL-0150994)

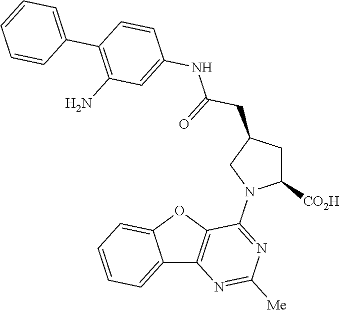 [SMILES: Cc1nc(N2C[C@@H](CC(=O)Nc3ccc(-c4ccccc4)c(N)c3)C[C@H]2C(=O)O)c2oc3ccccc3c2n1]

¹HNMR (400 MHZ, CD₃OD) δ 8.12 (br d, 1H), 7.65 (br d, 2H), 7.37 ˜7.50 (m, 5H), 7.30˜7.35 (m, 1H), 7.20 (d. J=<1 Hz, 1H), 7.01 (d. J=8 Hz, 1H), 6.92 (two d, 1H), 5.23 (m. 1H), 4.73-4.44 (m, 1H), 3.87-3.54 (m, 1H), 3.01-2.51 (m, 7H), 1.87 (m, 1H); (ESI) m/z calcd for C₃₀H₂₇N₅O₅: 521.21. Found: 522.31(M+H)⁺.

Compound 63: (2S,4R)-1-(2-methylbenzofuro[3,2-d]pyrimidin-4-yl)-4-(2-oxo-2-((2-(trifluoromethyl)-[1,1′-biphenyl]-4-yl)amino)ethyl)pyrrolidine-2-carboxylic acid (BBL-0100746)

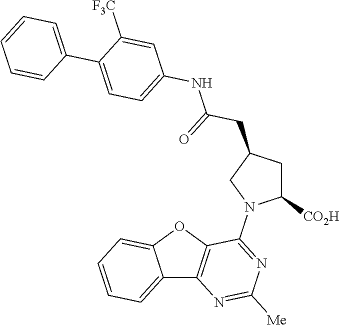 [SMILES: Cc1nc(N2C[C@@H](CC(=O)Nc3ccc(-c4ccccc4)c(C(F)(F)F)c3)C[C@H]2C(=O)O)c2oc3ccccc3c2n1]

¹HNMR (500 MHz, DMSO-d₆) δ 8.18 (d, J=1 Hz, 1H), 8.07 (d, 1H), 7.88 (d, 1H), 7.79 (br, 1H), 7.66 (br, 1H), 7.51 (d. 1H), 7.37˜7.50 (m, 3H), 7.35 (d. 1H), 7.30 (d, 2H), 5.14 (br s, 1H), 4.64-4.29 (m, 1H), 3.88 (m, 1H), 3.01-2.51 (m, overlapped with DMSO, 7H), 1.90-1.75 (m, 1H); (ESI) m/z calcd for C₃₁H₂₅F₃N₄O₄: 574.18. Found: 575.61(M+H)⁺.

Compound 64: (2S,4R)-1-(2-methylbenzofuro[3,2-d]pyrimidin-4-yl)-4-(2-oxo-2-((6-phenylpyridazin-3-yl)amino)ethyl)pyrrolidine-2-carboxylic acid (BBL-0150989)

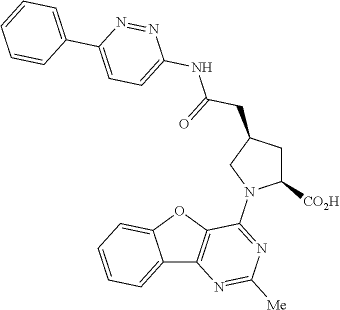 [SMILES: Cc1nc(N2C[C@@H](CC(=O)Nc3ccc(-c4ccccc4)nn3)C[C@H]2C(=O)O)c2oc3ccccc3c2n1]

¹HNMR (500 MHz, CD₃OD) δ 8.53 (d, 1H), 8.18 (s, 1H), 8.11 (m, 2H), 8.02 (d, 2H), 7.63 (m, 2H), 7.45˜7.55 (m, 3H), 7.41 (m, 1H), 5.25 (br s. 1H), 3.81-3.57 (m, 1H), 2.93 -2.83 (m, 5H), 2.56 (s, 3H), 2.00 (m, 1H); (ESI) m/z calcd for C₂₈H₂₄N₆O₅: 508.19. Found: 509. 33 (M+H)⁺.

Compound 65: (2S,4R)-1-(2-methylbenzofuro[3,2-d]pyrimidin-4-yl)-4-(2-oxo-2-((2-phenylpyrimidin-5-yl)amino)ethyl)pyrrolidine-2-carboxylic acid (BBL-0150990)

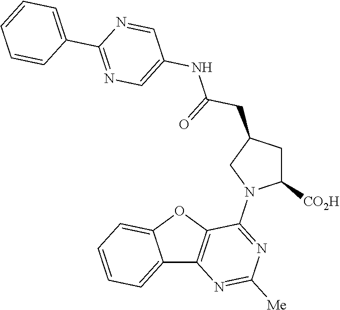 [SMILES: Cc1nc(N2C[C@@H](CC(=O)Nc3cnc(-c4ccccc4)nc3)C[C@H]2C(=O)O)c2oc3ccccc3c2n1]

¹HNMR (500 MHz, CD₃OD) δ 9.10 (s, 2H), 8.34 (m, 2H), 8.11 (d, 1H), 7.6 ˜7.75 (m. 2H), 7.46 (m, 4H), 5.25 (br s, 1H), 4.74-4.35 (m, 1H), 3.89 (m, 1H), 3.01-2.53 (m, 7H), 2.04-1.89 (m, 1H); (ESI) m/z calcd for C₂₈H₂₄N₆O₄: 508.19. Found: 509.33 (M+H)⁺.

Compound 66: (2S,4R)-4-(2-((2-fluoro-[1,1′-biphenyl]-4-yl)amino)-2-oxoethyl)-1-(2-methylbenzofuro[3.2-d]pyrimidin-4-yl)pyrrolidine-2-carboxylic acid (BBL-0150995)

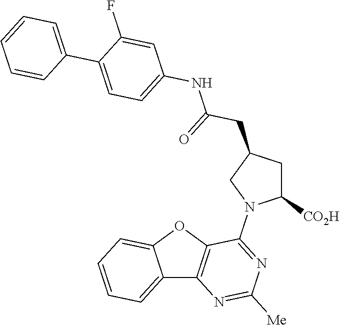 [SMILES: Cc1nc(N2C[C@@H](CC(=O)Nc3ccc(-c4ccccc4)c(F)c3)C[C@H]2C(=O)O)c2oc3ccccc3c2n1]

¹HNMR (500 MHZ, CD₃OD) δ 8.11 (d. J=10 Hz, 1H), 7.66 (m, 3H), 7.53 (d, J=10 Hz, 2H). 7.4˜7.5 (m, 4H), 7.35 (m, 2H), 5.24 (m, 1H), 4.75-4.44 (m, 1H), 3.88-3.57 (m, 1H), 3.01-2.52 (m. 7H), 2.15-1.88 (m, 1H); (ESI) m/z calcd for C₃₀H₂₅FN₄O₅: 524.19 Found: 525.27 (M+H)⁺.

Compound 67: (2S,4R)-1-(2-methylbenzofuro[3,2-d]pyrimidin-4-yl)-4-(2-oxo-2-((5-phenylpyridin-2-yl)amino)ethyl)pyrrolidine-2-carboxylic acid (BBL-0150969)

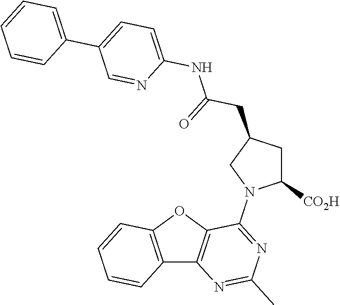 [SMILES: Cc1nc(N2C[C@@H](CC(=O)Nc3ccc(-c4ccccc4)cn3)C[C@H]2C(=O)O)c2oc3ccccc3c2n1]

### Example 3. cGAS Inhibitor Development

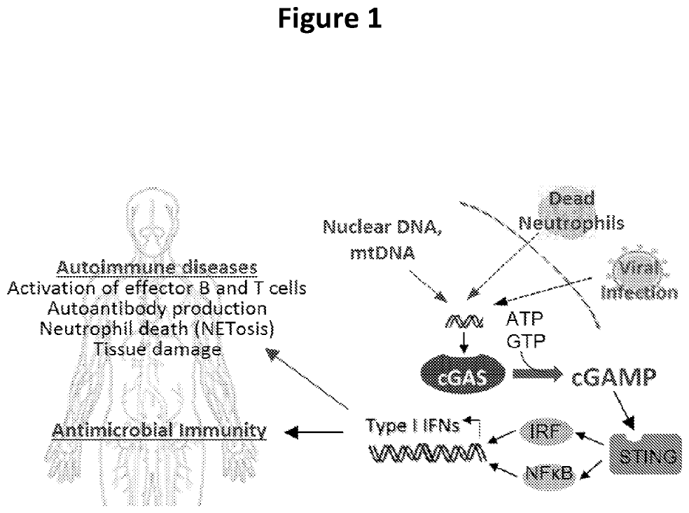

Detection of foreign nucleic acids is an important first line of defense in the immune response to microbial pathogens. However, aberrant induction of type I interferons (IFN) by self-nucleic acids causes devastating autoimmune diseases such as AGS. SLE and Sjogren's syndrome (FIG. 1). Type I IFNs (IFN-I) are strongly implicated in the pathogenesis of SLE and approximately two thirds of SLE patients have a blood interferon (IFN) signature. Plasmacytoid dendritic cells (pDCs) are the most prolific producers of type I IFNs, and their continuous stimulation is a major driver of SLE progression.

A key molecular trigger for nucleic acid-driven type I IFN induction is production of the unique cyclic dinucleotide, cGAMP, by the cytosolic DNA sensor, cGAS. The cGAS apoenzyme is enzymatically inactive; binding of non-specific dsDNA induces a transition to an active conformation that catalyzes the formation of cGAMP from ATP and GTP. cGAMP binds to the STING (stimulator of interferon genes) receptor to initiate the signaling for induction of type I IFNs. Knockout studies in animal models have clearly indicated that inhibiting cGAS is a promising approach for therapeutic intervention in monogenic type I interferonopathies such as AGS and, by extension, complex diseases such as SLE.

For example, studies in mice have established compelling support for targeting cGAS to block type I IFN production in SLE and AGS; both diseases are characterized by high levels of circulating type I IFNs and autoantibodies to nucleic acids and other nuclear antigens. 90% of AGS patients carry mutations in one of five different DNA modifying enzymes that result in accumulation of cytoplasmic DNA, most notably the dsDNA exonuclease Trex1 (23%) or RNase H2 (53%), which removes RNA from DNA:RNA hybrids. Knocking out these nucleases causes lethal autoimmune disease in mice. Genetic ablation of cGAS or STING in the nuclease-deficient mice protects against lethality and eliminates the autoimmune phenotypes, including interferon stimulated gene (ISG) induction, autoantibody production, and T-cell activation.

Mutations that impair the function of RNAse H₂, Trex1, and other nucleic acid modifying enzymes also occur with low frequency in SLE, including the TREX1 D18N mutation that causes familial chilblain lupus. Though less lethal than knocking out TREX1. TREX1 D18N mice have lupus-like inflammatory disease and almost half die within several months; knocking out a single cGAS allele drastically improves symptoms and survival, and disease is cured in the cGAS double knockout mice, including restoration of normal ISG expression and elimination of anti-DNA and anti-nuclear antibodies. However, the TREX1 D18N mouse does not have cutaneous symptoms.

Blocking cGAS would likely affect the immune response to some viral and bacterial infections, however, evidence suggests that a suitable balance between immune suppression and efficacy would be possible. First, knocking out a single copy of cGAS in mouse models of AGS and lupus results in a drastic improvement in autoimmune symptoms and survival. Second, there is considerable redundancy in the innate immune response to dsDNA from microbial pathogens: at least three additional pattern recognition receptors, IFI16, AIM2, TLR₉,respond to dsDNA. In addition, the immune system responds to multiple pathogen associated molecular patterns from a single pathogen; e.g., LPS, peptidoglycan and DNA from gram negative bacteria and RNA and DNA from retroviruses. Third, the Mn-sensitivity of cGAS inhibitors may be leveraged to provide greater potency in an autoimmune context relative to an antimicrobial context.

Several novel cGAS inhibitors were discovered using a cGAS HTS (i.e., high throughput screen) assay having favorable structural, physicochemical and ADME/PK properties that function via distinct mechanisms. The present inventors also determined that a physiological cGAS effector molecule (Mn²⁺) profoundly affects the potency of the disclosed compounds, which can inform development of cGAS drugs with more specific effects on autoimmune pathogenesis and less impact on anti-microbial immunity.

Structure-driven ligand optimization was used to advance the disclosed compounds by testing efficacy using SAR and structural models. Structure driven ligand optimization and MOA analysis was performed for the disclosed compounds using human and mouse cGAS to provide compounds having an IC₅₀≤100 nM with human cGAS and ≤500 nM with mouse cGAS, and an IC₅₀≥10 μM off target (e.g., Kinases, GTPases, PDEs, OAS's).

In short, the present inventors have produced co-crystals of human cGAS lacking the unstructured N-terminal domain with Compounds A, 5, and BBL0100243 ((2-methylbenzofuro[3,2-d]pyrimidin-4-yl)-L-proline) (data not shown). The tricyclic cores of these compounds binds in the active site where the adenosine of ATP binds and. surprisingly, they induce formation of a substantial pocket adjacent to the active site. The analysis of the structure of compound A found that there is substantial room to build into the ligand-induced pocket and that there are opportunities for hydrogen bonding with one or more amino acid side chains or backbone amides at the back of the pocket. The structural data from the compound 5 co-crystal confirms this strategy and in addition indicates that there is additional flexibility in the induced pocket. These results suggest that the compounds have allosteric binding properties, at least over a short distance, and may stabilize an inactive cGAS conformation, properties vital in developing a highly selective drug with a long residence time. Additionally, the structural information from the co-crystals allowed the inventors to design analogs with non-polar interactions in the ligand-induced pocket and physicochemical properties favoring membrane permeability.

SAR-driven medicinal chemistry was used to design the compounds of the disclosure and increase the potency into the nanomolar range. Specifically, the compounds were designed to increase non-polar and hydrogen bonding interactions, especially within the ligand-induced pocket and to impart physicochemical properties known to increase cellular permeability and oral bioavailability, primarily maintaining lipophilicity, and minimizing polar surface area and conformational flexibility. The design efforts of compounds was biased toward allosteric inhibitors because allosteric drugs often have longer residence times and greater selectivity as compared with purely competitive drugs.

The following criteria was developed to evaluate the compounds of this disclosure:

1. Biochemical potency and selectivity: IC₅₀≤100 nM in cGAS enzymatic assay and IC₅₀≥50 μM off-target.
2. Cellular activity: IC₅₀ of 1μM for inhibition of type I IFN expression in monocytes and primary human cells and evidence of target binding in cells by cellular thermal shift assay (CETSA).
3. ADME Properties: mouse and human microsomal stability t_{1/2}>60 min, kinetic aqueous solubility >100 μg/mL, Caco-2 and MDCK-MDR₁ permeability A→B >1×10⁻⁶, efflux ratio <2.5.

With this structure driven approach, the inventors have produced a variety of analogs with significant improvements in biochemical (˜100 nM) and cellular (<1 μM) potency, and promising ADME properties (Table 1). The inventors have found that the combined structural and biochemical results suggest that the compounds of this disclosure have a mechanism similar to type II, or DGF-out, kinase inhibitors such as imantinib mesylate (GLEEVEC ®, Novartis Pharmaceuticals, Basel, Switzerland) that bind to the ATP site and extend into an induced hydrophobic pocket, stabilizing an inactive conformation.

### Example 4. Adsorption, Distribution, Metabolism, and Excretion Profile

ADME studies: Compound A and the compounds of the disclosure were tested for aqueous solubility (KSOL), metabolic stability (human and mouse liver microsomes) and permeability (Caco-2 and/or MDR1-MDCK cells) to provide an initial indication of oral bioavailability.

The compounds of the disclosure generally have physicochemical properties that are predictive of good oral bioavailability (Table 1). None of the analogs shown in Table 1 have any Lipinski violations, with the exception of several compounds that are slightly over 500 Da in MW. However, many recently approved drugs exceed the 500 Da Lipinski rule. In addition to the Lipinski parameters, numerous studies on the properties of approved drugs and new chemical entities that failed in development have indicated that the number of rotatable bonds (RB), total polar surface area (TPSA) and lipophilicity (logD) are key determinants of oral bioavailability. For example, 81% of drugs with >20% oral bioavailability in humans have a TPSA <140 Å2 and an RB <10. All of the compounds in Table 1 meet these criteria as well.

The favorable physicochemical properties are reflected in promising results in in vitro ADME tests. Kinetic solubility (K_{SOL}) in PBS, pH 7.4, was measured for several compounds of the disclosure, and found to be greater than 100 μM for most compounds. Metabolic stability, which was routinely measured with mouse and human hepatocytes, is outstanding, with t, greater than 2 hours for all of the compounds of the disclosure in which it has been measured. Cell permeability was measured using MDCK cells expressing human MDR₁ (P-glycoprotein) to assess passive permeability as well as active transport out of the cell. Passive membrane permeability (Papp) greater than 1×10⁻⁶ cm/sec and low efflux ratio (Papp(B-A)/Papp(A-B)) less than 2.5 are generally considered to be indicative of good intestinal absorption. Numerous compounds in Table 1 meet these permeability criteria, which is consistent with their design for TPSA <140 Å² and increased lipophilicity.

Almost half of the compounds have less than 10-fold lower potency in cellular assays than in cGAS enzymatic assays under physiological conditions (at 1 mM ATP/GTP). (i.e., cellular IC₅₀ is less than 10-fold greater than biochemical IC₅₀), which is a smaller difference than for many kinase inhibitors. However, for Compound 5 and 63 the biochemical/cellular potencies are essentially identical, which is probably attributable to their higher lipophilicity and cell permeability (see logD and MDCK data in Table 1) and is very encouraging from an overall ligand design perspective. The increased affinity of Compounds 4 and 5, which incorporated hydrophobic bicyclic moieties linked to the pyrrolidine, further validates the structure driven approach.

The MDCK-MDR₁ permeability A ->B is shown in Table 2 below, where A represents 5-10 cm/s×10⁻⁶; B represents 1-5 cm/s×10⁻⁶; and C represents <1 cm/ss×10⁻⁶. The metabolic stability half time, in both mouse and human hepatocytes, is also shown in Table 2 below, where A′ represents 100-200 minutes; B′ represents 200-300 minutes; and C′ represents >300 minutes.

TABLE 1
Compound Properties

| | MDCK-MDR | | | | | | |
|---|---|---|---|---|---|---|---|
| | Permeability | Mouse MS | Human MS | | | | |
| Cmpd | A→B | Stability | Stability | Kₛₒₗ | | TPSA | Rotatable |
| No. | (cm/s × 10⁻⁶) | t_{1/2} (min) | t_{1/2} (min) | (μM) | log D | (Å²) | bonds |
| A | C | A′ * | A′ * | 542 | 0.28 | 121.45 | 5 |
| 1 | B | A′ | C′ | 487 | 1.51 | 124.35 | 5 |
| 2 | C | C′ | C′ | 506 | 0.93 | 99.77 | 5 |
| 3 | C | C′ | | 496 | 1.05 | 137.24 | 5 |
| 4 | B | A′ | | 488 | 1.41 | 121.45 | 5 |
| 5 | A | C′ | A′ | 8 | 3 | 108.56 | 6 |
| 6 | C | C′ | C′ | 496 | 0.39 | 130.68 | 6 |
| 7 | B | | | | 2.39 | 111.8 | 6 |
| 8 | C | | | | 1.19 | 123.83 | 6 |
| 9 | C | | | | 1.3 | 115.04 | 6 |
| 10 | C | | | | 1.29 | 137.24 | 6 |
| 11 | C | | | | 1.37 | 137.24 | 6 |
| 12 | A | | | | 2.88 | 117.79 | 7 |
| 13 | C | | | | 1.11 | 117.79 | 6 |
| 14 | C | | | | 1.03 | 117.79 | 6 |
| 15 | B | | | | 1.88 | 108.56 | 5 |
| 16 | B | | | | 1.88 | 108.56 | 5 |
| 17 | C | | | | 1.84 | 108.56 | 5 |
| 18 | C | | | | 1.43 | 108.56 | 5 |
| 19 | B | | | | 1.42 | 108.56 | 5 |
| 20 | B | | | | 1.56 | 108.56 | 5 |
| 21 | B | | | | 1.88 | 108.56 | 5 |
| 22 | B | | | | 1.87 | 108.56 | 5 |
| 23 | B | | | | 1.96 | 108.56 | 5 |
| 24 | B | | | | 2.19 | 108.56 | 6 |
| 25 | B | | | | 2.18 | 108.56 | 6 |
| 26 | A | C′ | C′ | 80 | 2.39 | 108.56 | 5 |
| 27 | B | C′ | A′ | 548 | 1.51 | 108.56 | 5 |
| 28 | C | | | | 1.12 | 137.24 | 5 |
| 29 | C | | | | 0.94 | 137.24 | 5 |
| 30 | C | | | | 1.05 | 137.24 | 5 |
| 31 | A | C′ | A′ | 313 | 2.41 | 108.56 | 5 |
| 32 | C | B′ | C′ | 527 | 1.54 | 121.45 | 5 |
| 33 | C | | | | 1.25 | 121.45 | 5 |
| 34 | B | | | | 1.25 | 121.45 | 5 |
| 35 | B | | | | 1.54 | 121.45 | 5 |
| 36 | B | C′ | C′ | 155 | 3.07 | 108.56 | 6 |
| 37 | C | B′ | C′ | 547 | 1.79 | 121.45 | 6 |
| 38 | C | C′ | A′ | 84 | 2.7 | 137.24 | 6 |
| 39 | | | | | 2.69 | 137.24 | 6 |
| 40 | | | | | 2.27 | 147.47 | 6 |
| 41 | | | | | 2.03 | 147.47 | 6 |
| 42 | | | | | 2.22 | 147.47 | 6 |
| 43 | | | | | 1.88 | 147.47 | 6 |
| 44 | C | | | | 0.99 | 134.34 | 6 |
| 45 | | | | | 0.62 | 134.34 | 6 |
| 46 | C | C′ | A′ | 42 | 2.69 | 137.24 | 6 |
| 47 | | | | | 1.26 | 137.66 | 6 |
| 48 | | | | | 1.26 | 137.66 | 6 |
| 49 | | | | | 1.46 | 137.24 | 6 |
| 50 | A | | | | 2.8 | 117.79 | 7 |
| 51 | | | | | 2.72 | 128.79 | 6 |
| 52 | | | | | 0.92 | 138.1 | 6 |
| 53 | | | | | 0.94 | 132.03 | 6 |
| 54 | B | A′ | A′ | 285 | 3.45 | 108.56 | 7 |
| 55 | | | | | 2.34 | 121.45 | 7 |
| 56 | C | | | | 2.15 | 121.45 | 7 |
| 57 | C | | | | 1.29 | 121.03 | 6 |
| 58 | C | | | | 0.92 | 132.03 | 6 |
| 59 | | | | | 2.24 | 128.79 | 7 |
| 60 | | | | | 2.24 | 128.79 | 7 |
| 61 | | | | | 2.75 | 117.79 | 7 |
| 62 | | | | | 2.24 | 134.58 | 6 |
| 63 | B | >360 | A′ | 570 | 3.82 | 108.56 | 7 |
| 64 | | | | | 1.47 | 134.34 | 6 |
| 65 | | | | | 1.61 | 134.34 | 6 |
| 66 | | | | | 3.06 | 108.56 | 6 |
| 67 | A | | | | 2.26 | 121.45 | 6 |

TPSA: Topological polar surface area;
logD: lipophilicity;
MS: metabolic stability in hepatocytes (except * in liver microsomes);
MDCK-MDR: Madin-Darby Canine Kidney cells-Multi Drug Resistance pump

### Example 5. cGAS Inhibitor Enzymatic Assays

Structural, biochemical and biophysical analysis and selectivity profiling: Potency and MOA studies, including Mn²⁺ sensitivity, were performed using the Transcreener cGAS enzymatic assay, which is manufactured at BellBrook Labs (Fitchburg, Wisconsin, USA). This homogenous cGAS enzymatic assay was developed with fluorescence polarization (FP) and time-resolved Forster resonance energy transfer (TR-FRET) readouts, as described in International Patent Publication WO 2020/142729, incorporated by reference herein in its entirety. Plates are read on a PHERAstar FSX multimode reader (BMG). The compounds of the disclosure were tested for inhibition of cGAS (30 nm) using this cGAS enzymatic assay under standard conditions (100 μM ATP and GTP, 62.5 nM bp ISD, 60 minute reactions), high ATP and GTP (1 mM) to mimic physiological conditions, in the presence of 200 μM MnCl₂, and with mouse cGAS under standard conditions.

The release of MnCl₂ from organelles into the cytoplasm can play a critical role in initiating a cGAS-dependent anti-viral immune response, both in cells and in mice: Mn²⁺ binding to cGAS stimulates production of cGAMP in the presence of very low concentrations of dsDNA that would otherwise be non-stimulatory. The inventors confirmed that Mn²⁺ increases sensitivity to DNA and found that the effect is inversely related to DNA length, ranging from 5-fold for a 40 mer to 40-fold for a 15 mer (data not shown), indicating that human cGAS can be activated by shorter DNA fragments than previously thought, similar to mouse cGAS. Accordingly, effect of Mn²⁺ on pharmacological modulation of cGAS was tested. Known human cGAS inhibitors (the antimalarial quinacrine and PF06928215) were shown to be significantly less potent when Mn²⁺ was present at a physiological concentration (200μM), with increases in IC₅₀ as much as 10-fold. The disclosed compounds were also negatively-sensitive to Mn²⁺, with IC₅₀ shifts ranging from 4- to 10-fold for different analogs (data not shown). Without wishing to be bound by theory, it is hypothesized that the inverse Mn-dependence of the cGAS antagonists can be leveraged to provide an enhanced therapeutic window by blocking cGAS more effectively under pathogenic conditions while having less of an effect on the response to microbial pathogens.

The IC₅₀ values for FP under standard and under physiological conditions were determined for several exemplary compounds of the invention. The relative activities for FP Standard IC₅₀ are shown in Table 2 below, where A represents <50 nM; B represents 50-100 nM; C represents 100-200 nM; D represents 200 nM-1μM; and E represents 1-10 μM. For the relative activities for FP Physiological IC₅₀, A′ represents <200 nM; B′ represents 200-500 nM; C′ represents 500 nM-1μM; D′ represents 1-5 μM; and E′ represents >5 μM.

TABLE 2
Enzymatic Assays

IC₅₀ (μM)

| Cmpd No. | FP Standard | FP Physiological |
|---|---|---|
| 1 | B | D′ |
| 2 | B | C′ |
| 3 | E | E′ |
| 4 | B | A′ |
| 5 | B | C′ |
| 6 | B | D′ |
| 7 | D | D′ |
| 8 | E | E′ |
| 9 | E | E′ |
| 10 | A | B′ |
| 11 | B | B′ |
| 12 | D | E′ |
| 13 | D | D′ |
| 14 | E | E′ |
| 15 | D | D′ |
| 16 | D | D′ |
| 17 | D | E′ |
| 18 | D | D′ |
| 19 | D | D′ |
| 20 | D | E′ |
| 21 | C | D′ |
| 22 | C | D′ |
| 23 | D | E′ |
| 24 | D | D′ |
| 25 | D | E′ |
| 26 | D | D′ |
| 27 | C | D′ |
| 28 | D | D′ |
| 29 | C | C′ |
| 30 | C | D′ |
| 31 | B | B′ |
| 32 | B | D′ |
| 33 | C | D′ |
| 34 | A | B′ |
| 35 | A | C′ |
| 36 | B | B′ |
| 37 | A | A′ |
| 38 | A | B′ |
| 39 | C | C′ |
| 40 | A | B′ |
| 41 | C | C′ |
| 42 | B | C′ |
| 43 | C | C′ |
| 44 | A | B′ |
| 46 | A | A′ |
| 47 | C | D′ |
| 48 | A | C′ |
| 49 | C | C′ |
| 50 | A | B′ |
| 51 | A | A′ |
| 52 | C | D′ |
| 53 | D | D′ |
| 54 | B | C′ |
| 56 | D | D′ |
| 57 | C | D′ |
| 58 | D | D′ |
| 59 | A | C′ |
| 60 | A | B′ |
| 63 | C | B′ |
| 64 | D | D′ |
| 65 | B | C′ |
| 67 | B | C′ |

More than one third of the compounds have ICso values of 100 nM or lower, as measured by the cGAS FP assay under standard conditions, with several in the 10-20 nM range. Most of the others have potencies below 1μM; a few have potencies below 5μM. Potency in the presence of saturating concentrations of ATP and GTP (1 mM each) has increased to an even greater extent to less than 1 μM for approximately half of the compounds and less than 200 nM for several compounds. This is important because, like with kinase inhibitors, cGAS active site inhibitors must compete with millimolar concentrations of ATP and GTP in the cytoplasm.

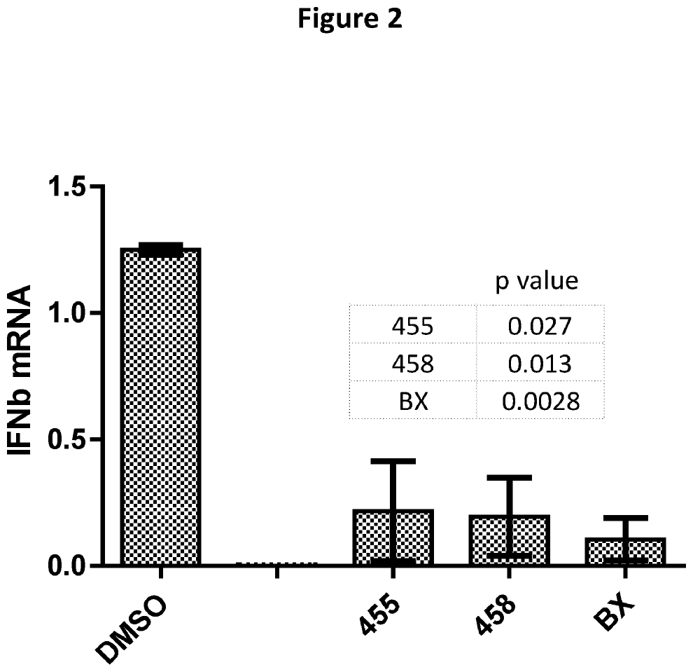

Long residence times, i.e., slow dissociation, can also lead to improved cellular activity because equilibrium with competing molecules in the cytoplasm is slowed. The cGAS enzymatic assay with the jump dilution method was used to measure inhibitor residence times (1/K_{off}). Dissociation half-times of less than 10 min were observed for early compounds, whereas some more recent compounds have half times as high as two hours (FIG. 2). These results are encouraging, as a residence time of 2-3 hours can have a significant positive impact on pharmacokinetics and pharmacodynamics.

Potency with mcGAS is important for vivo efficacy studies and represents a challenge for lead development because mcGAS shares only 57% amino acid identity with human enzyme and there are significant functional differences. The exemplary compounds initially had undetectable inhibition of mcGAS, but IC₅₀ values less than 10 μM were observed for several compounds, such as 1-5; e.g., 8.3 and 4.23 μM for Compounds 4 and 5, respectively (data not shown).

The compounds selectivity was tested using a panel of nucleotide utilizing enzymes that are functionally related to cGAS and/or are in the cGAS/STING pathway: TBK1, IKKB. OAS1, ENPP1, and PDE4. Using enzymatic assays based on homogenous immunodetection of AMP or ADP, alone or in combination with coupling enzymes, no inhibition of any of the off-target enzymes were observed in dose response experiments with a maximum concentration of 50μM (data not shown).

### Example 6. Cellular Studies to Demonstrate Target Engagement, Blocking of CGAS-STING Pathway, and Therapeutic Efficacy

Cellular assays: The human monocyte cell line THP-1 and human primary PBMCs were used to evaluate the cellular activity of compounds with good biochemical potency. These cells produce a robust cGAS/STING-dependent type I IFN response when stimulated with dsDNA and other pathogen-associated molecular patterns, which was detected using a standard ELISA for IFNβ (R&D Systems). The TBK1 inhibitor, BX-795, which acts downstream of cGAS/STING, was used as a probe.

CETSA was also used to confirm that the compounds are binding to cGAS in cells; THP-1 cells are used for this analysis. Compounds were tested in dose response mode by incubating with cells for 1.5 hours at 37° C., followed by pelleting and re-suspending in PBS, heating to 51.5° C. for 3 min and cooling to room temp. Cells were then lysed, debris, including denatured cGAS, was pelleted and the supernatant was analyzed for soluble cGAS by Western Blot using anti-cGAS primary Ab (Cell Signaling). Band intensity was analyzed using Image J software. Stimulation with cGAMP directly activates STING, circumventing cGAS; this was used to determine if compounds had effects on downstream components of cGAS/STING signaling. The IFN-6 ELISA was used as the primary measure of cellular potency and selectivity (Table 3) and used the reporter gene assays for assessing off-target activity with other pattern recognition receptors.

The ICso values for IFNB ELISA stimulated with THP-1, PBMC, DNA, and cGAMP were determined for several exemplary compounds of the invention, and are provided in Table 3 where A represents <1 μM; B represents 1-2.5 μM; C represents 2.5-10 μM; D represents 10-20 μM; and E represents >20 μM.

TABLE 3
Assay Results

IC₅₀ (μM)
IFNβ ELISA stimulated with

| Cmpd No. | THP-1 | PBMC |
|---|---|---|
| A | E | |
| 1 | C | |
| 2 | E | |
| 3 | D | |
| 4 | E | |
| 5 | A | B |
| 6 | C | |
| 7 | D | D |
| 8 | E | |
| 9 | E | |
| 10 | E | |
| 11 | E | |
| 12 | B | C |
| 13 | D | |
| 14 | E | |
| 15 | E | |
| 16 | E | |
| 17 | E | |
| 18 | E | |
| 19 | D | |
| 20 | E | |
| 21 | D | |
| 22 | C | D |
| 23 | E | |
| 24 | D | D |
| 25 | D | |
| 26 | C | C |
| 27 | C | C |
| 28 | E | |
| 29 | E | |
| 30 | E | |
| 31 | B | B |
| 32 | C | D |
| 33 | C | D |
| 34 | C | C |
| 35 | C | C |
| 36 | B | B |
| 37 | B | B |
| 38 | B | C |
| 39 | C | C |
| 40 | D | E |
| 41 | E | E |
| 42 | E | E |
| 43 | E | E |
| 44 | E | |
| 46 | A | B |
| 49 | E | |
| 50 | A | |
| 51 | D | E |
| 53 | E | |
| 54 | B | C |
| 56 | E | |
| 57 | E | |
| 58 | E | |
| 63 | A | B |
| 67 | C | |

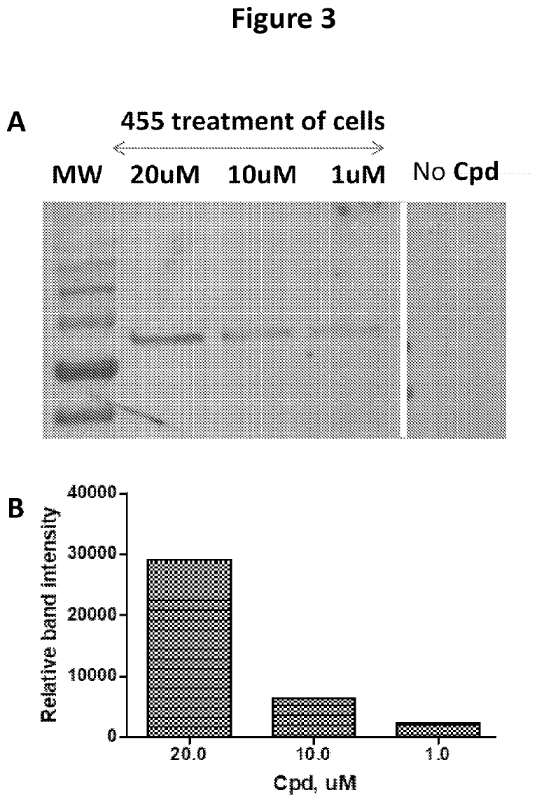

In dose response experiments in THP-1 cells using the IFNB ELISA, IC values of less than 10 μM were observed for approximately one third of the compounds and less than 1 μM for several. Importantly, inhibition observed of IFNβ expression in DNA-stimulated cells was 3-10 fold more potent than in cGAMP-stimulated cells. These results clearly indicate selective inhibition of cGAS-driven IFN-β expression. It was confirmed that representative compounds inhibited IFN-B mRNA expression using RT-PCR (FIG. 2). The reporter gene assays were used to assess off-target activity with IRF30Luc (RIG-1) or NFKB (TLR4); CellTiter Glo (ATP levels) and Presto Blue (reducing equivalents) were used to assess cytotoxicity; all assays were performed in dose response mode. Compound 5 showed some off-target activity with the TLR₄ pathway and cytotoxicity at concentrations above 25 μM; whereas Compound 4, showed no detectable inhibition of RIG-1 or TLR₄ signaling and no cytotoxicity at concentrations as high as 100 μM (data not shown). Compound 4 also inhibited the isolated Luc or SEAP reporter enzymes appreciably, which inhibited Luc 50% at 80 μM (data not shown). The inventors recently began using human PBMCs to assess cellular potency in a more physiologically relevant context. Many compounds showed dose dependent inhibition of IFN-β production in PBMCs with IC₅₀ values of 10 μM or lower. Lastly, the inventors were able to demonstrate intracellular target engagement for Compound 5 in THP-1 cells using a cellular thermal shift assay (CETSA) (FIG. 3). Compound 4 also stabilized cGAS in CETSA, but the concentration dependence was not as clear.

Example 7. Mouse model of cGAS-driven type I IFN induction

In lupus and related autoimmune diseases, cGAS is activated by DNA released from dying cells and the resulting type I IFN production drives inflammation and tissue damage. Studies have shown that oxidized DNA (oxDNA) containing 8-hydroxyguanosine (8-OHG), which is present in microparticles formed by dying cells in the serum of lupus patients, is a more potent stimulator of cGAS-driven type I IFN induction in mice than unmodified DNA, as measured by type I IFN levels in plasma and expression of interferon sensitive gene (ISG) mRNA. Therefore, testing cGAS antagonists for their ability to block type I IFN induction in mice that have been stimulated with oxDNA is a simple model for assessing their in vivo efficacy as lupus therapeutics. Because our cGAS antagonists have high potency with human cGAS and are more than 10-fold less active with mouse cGAS, we use humanized mice to test their in vivo efficacy; e.g., irradiated NSG—SGM3 mice engrafted with human hematopoietic stem cell (CD34+) to reconstitute a human immune system.

Study design. The study includes one test compounds, a vehicle control (DMSO). and an untreated (no stimulation with oxDNA) mouse as positive control, 7 female 12-31 week old HuCD34—NCG+for each group, total of 21 mice. Note that female mice are used because of the much more pronounced ISG upregulation observed relative to males in the UVB model and the much higher prevalence of lupus in women relative to men. OxDNA will be produced by irradiation with UVC light as previously described. Test compounds are administered orally at a dose of 30-60 mg/kg two hours prior to injection with oxDNA. An additional dose may be administered immediately following stimulation with oxDNA if necessary to achieve sufficient serum levels of test compound. Mice are injected intravenously with 20 μg of oxDNA combined with 100 μg of DOTAP at time =0. On study hour 3, blood is collected among all groups via the submandibular vein, plasma is prepared. and stored at −80° C. for INFB multiplex analysis. On study hour 6, animals are culled, and the blood is collected by cardiac puncture and spleens are harvested for ISG mRNA expression analysis.

Endpoints includes ISG mRNA expression in spleen and IFN-β in plasma.

ISG mRNA expression and IFN-β levels. mRNA is extracted from spleens, cDNA is synthesized, and ISG transcripts, selected based on previous studies of IFN response to UV. is quantified by real time quantitative PCR (qPCR) and normalized to Gapdh transcript levels. Fold induction in ISG expression is determined using the standard formula 2^{(-ΔΔCt)} relative to baseline, i.e., without oxDNA stimulation. IFNβ and other inflammatory cytokine levels in plasma is measured using Legendplex Mouse Inflammation Panel and quantified by FACS analysis. Reduction of plasma IFNβ levels and ISG mRNA expression levels in oxDNA-stimulated mice by more than 30% with an orally-dosed cGAS antagonist is a strong indicator that it may have therapeutic value for treatment of lupus and related autoimmune diseases.

### Example 8. Pharmacokinetic Evaluation of Compound 5 (BBL0100455)

The pharmacokinetic characteristics of Compound 5 (BBL-100455) were estimated in C57BL/6 female mice following intravenous (IV) bolus and oral (PO) administration.

In short, the compound of the disclosure at 0.6 mg/mL in PBS containing 5% MSO and 25% PEG-400 was administered by IV injection (3 mg/kg). For PO (30 mg/kg), the compound of the disclosure at 3 mg/mL in PBS containing 10% MSO and 50% PEG-400 was orally administered. At the given time points (0.083 h, 0.167 h, 0.25 h, 0.50 h 1 h, 2 h, 4 h, 7 h, 16 h and 24 h), blood samples were collected using heparinized calibrated pipettes. Samples were centrifuged at 15000 rpm for 10 min. Subsequently, blood plasma was collected from the upper layer. The plasma was frozen at −80° C. for later analysis. At 2 h. 7 h and 24 h, brain samples were collected and immediately stored at 80° C. for later analysis.

The analytical curve was constructed using ten non-zero standards with Compound 5 (BBL-100455) concentration ranging from 1 to 2500 ng/ml in the blank plasma and brain tissue. A blank sample (matrix sample processed without internal standard) was used to exclude contamination. The linear regression analysis of BBL- 100455 was performed by plotting the peak area ratio (y) against the BBL-100455 concentrations (x) in ng/ml. The linearity of the relationship between peak area ratio and concentration was demonstrated by the correlation coefficients (R) obtained for the linear regression of (r=>0.990 in all samples).

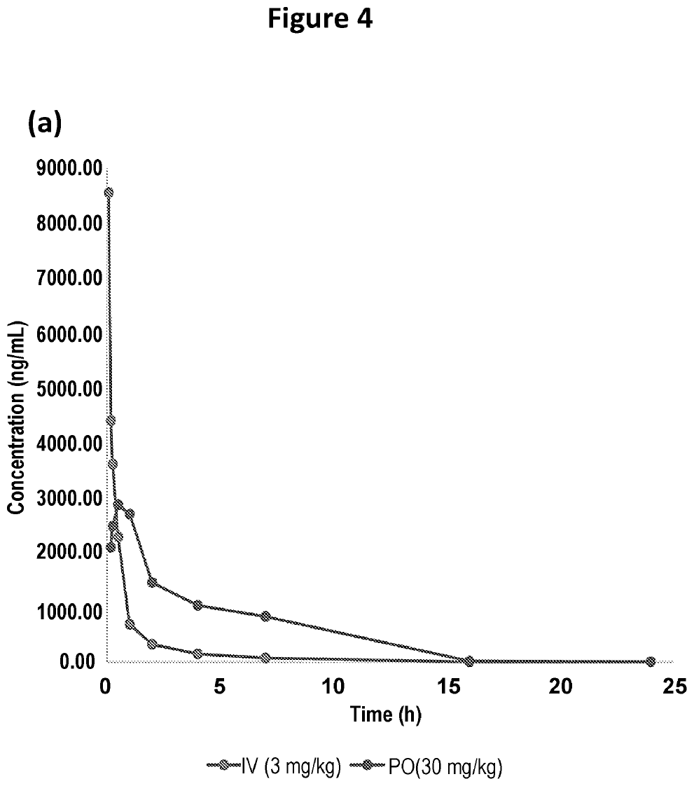

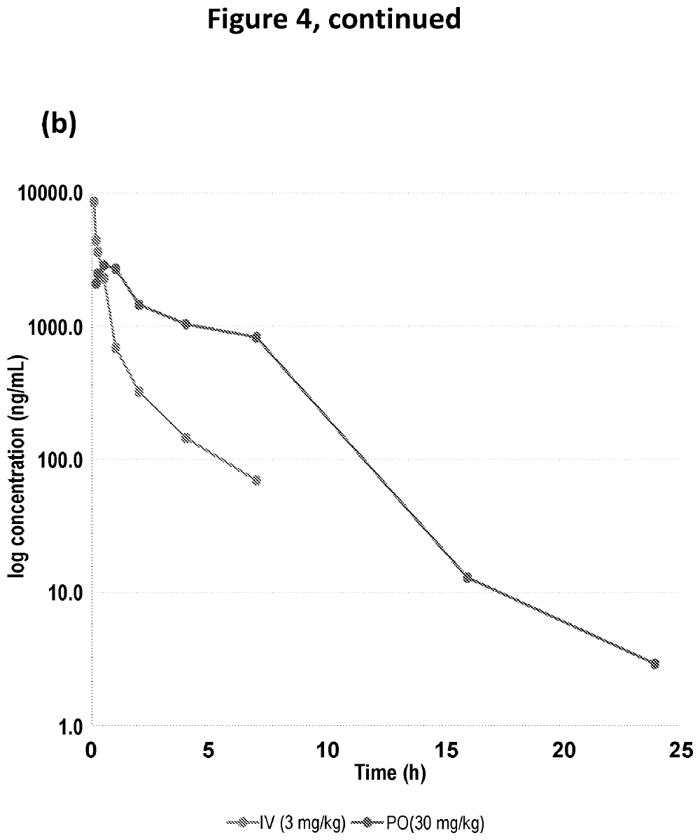

The individual and average compound 5 concentration-time data for IV and PO dosed groups are presented in FIG. 4 for plasma concentration. The pharmacokinetic parameters are listed in Table 4. All PK parameters were estimated using non-compartmental analyses with Phoenix/WINONLIN.

TABLE 4
PK parameters of compound 5 in plasma following IV and PO administration

| | Dose | Cₘₐₓ | Tₘₐₓ | AUC_{0-tidc} | AUC_{0-inf} | t_{1/2} |
|---|---|---|---|---|---|---|
| Route | (mg/kg) | (ng/mL) | (hr) | (hr*ng/mL) | (hr*ng/mL) | (hr) |
| IV | 3 | N/A | N/A | 4628.30 | 4857.95 | 2.30 |
| PO | 30 | 2866.67 | 0.50 | 13580.10 | 13589.50 | 2.25 |

| | Dose | CL | CL/F | Vss | Vz/F | |
|---|---|---|---|---|---|---|
| Route | (mg/kg) | (mL/hr/kg) | (mL/hr/kg) | (mL/hr/kg) | (mL/hr/kg) | % F |
| IV | 3 | 617.54 | NA | 853.2 | NA | NA |
| PO | 30 | NA | 2207.58 | N/A | 7177.6 | 29.3 |

Cₘₐₓ = Maximum observed concentration, Tₘₐₓ = Time to reach Cₘₐₓ, AUC_{0-tidc} = Area under the concentration-time curve from time zero to time of last detectable concentration, AUC_{0-inf} = Area under the concentration-time curve from time zero to infinite, CL = Systemic clearance, CL/F: Apparent clearance, Vss: Volume of distribution at steady state, Vz/F: Volume of distribution associated with the terminal elimination phrase, Terminal elimination half-life (t_{1/2}) was calculated based on data points (>=3) in the terminal phase with correlation of coefficient >0.90, % F = bioavailability.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A compound according to Formula (I): 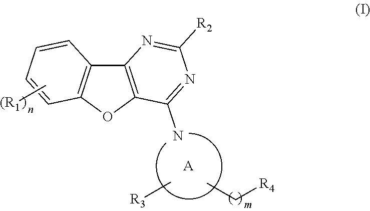
or a pharmaceutically acceptable salt, N-oxide, and/ or solvate or hydrate thereof, wherein:
m is an integer of 1, 2, or 3;
n is an integer of 0. 1, 2, 3, or 4;
ring A represents a 4 to 8 membered heterocyclyl ring;
each R₇ is independently selected from halogen, —NO₂, —CN, C₁-C₆ alkyl, C₁-C₆ haloalkyl. —OH, ₁-C₆ alkoxy, and C₁-C₆ haloalkoxy:
R₂ is hydrogen, ₁-C₆ alkyl, or ₁-C₆ haloalkyl;
R₃ is —CO₂R₅, —COR₅, —C(O)NR₅R₆, —CONH—OH, —S(O)₀₋₂—R₅, SO₂OR₅, or —SO₂NR₅R₆; and
RA is —C(O)NR₆R₇, —CO₂R₇, —SO₂OR₇, or —SONR₆R₇,
wherein
R₅ is hydrogen or C₁-C₆ alkyl;
R₆ is hydrogen, C₁-C₆ alkyl, or C₁-C₆ haloalkyl;
R₇ is selected from the group consisting of aryl(C₀-C₄ alkyl) optionally substituted with one or more R₈, heteroaryl(C₀-C₄ alkyl) optionally substituted with one or more R₉, heterocyclyl(C₀-C₄ alkyl) optionally substituted with one or more R₈, and cycloalkyl(C₀-C₄ alkyl) optionally substituted with one or more R₈;
each R₃ is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R₆, —C(O)OR₆, and —C(O)NR₅R₆, or two R₆ form an oxo;
each R₉ is independently selected from the group consisting of halogen, —NO₂, —CN, C₁-C₆ alkyl, C₁-C₆ haloalkyl, —Na, —NH₂, —NH(C₁-C₆ alkyl), —N(C₁-C₆ alkyl)₂, —OH, C₁-C₆ alkoxy, C₁-C₆ haloalkoxy, aryl(C₀-C₁ alkyl) optionally substituted with one or more R₁₀. heteroaryl(C₀-C₁ alkyl) optionally substituted with one or more R₁₀, heterocyclyl(C₀-C₁ alkyl) optionally substituted with one or more R₁₀, and cycloalkyl(C₀-C₁ alkyl) optionally substituted with one or more R₁₀, and
each R₁₀ is independently selected from the group consisting of halogen, —NO₂, —CN, C₁-C₆ alkyl, C₁-C₆ haloalkyl, —N₃, —NH₂, —NH(₁-C₆ alkyl), —N(C₁-C₆ alkyl)₂, —OH, C₁-C₆ alkoxy, C₁-C₆ haloalkoxy, and -methyl-OH;
provided the compound is not (2S,4R)-1-(2-methylbenzofuro[3,2-d]pyrimidin-4-yl)-4-(2-oxo-2-(pyridin-4-ylamino)ethyl)pyrrolidine-2-carboxylic acid, (2S,4R)-4-(2-((1H-pyrazol-4-yl)amino)-2-oxoethyl)-1-(2-methylbenzofuro[3,2-d]pyrimidin-4-yl)pyrrolidine-2-carboxylic acid, (2S,4R)-1-(2-methylbenzofuro[3,2-d]pyrimidin-4-yl)-4-(2-oxo-2-(pyridin-2-ylamino)ethyl)pyrrolidine-2-carboxylic acid, (2S,4R)-4-(2- (cyclopentylamino)-2-oxoethyl)-1-(2-methylbenzofuro[3,2-d]pyrimidin-4-yl)pyrrolidine-2-carboxylic acid; or (2S,4R)-1-(2-methylbenzofuro[3,2-d]pyrimidin-4-yl)-4-(2-oxo-2-(phenylamino)ethyl)pyrrolidine-2-carboxylic acid.

2. The compound according to claim 1, wherein n is 0, 1, or 2.

3. The compound according to claim 1, wherein n is 0 or 1.

4. The compound according to any of claims 1-3, wherein R₁ is independently selected from halogen, C₁-C₆ alkyl, C₁-C₆ haloalkyl, —OH, and C₁-C₆ alkoxy.

5. The compound according to any of claims 1-3, wherein R₁ is independently selected from C₁-C₃ alkyl, —OH, and C₁-C₃ alkoxy.

6. The compound according to claim 1, wherein n is 0.

7. The compound according to any one of claims 1-6, wherein R₂ is hydrogen or C₁-C₆ alkyl.

8. The compound according to any one of claims 1-6, wherein R₃ is hydrogen or C₁-C₄ alkyl.

9. The compound according to any one of claims 1-6, wherein R₂ is hydrogen.

10. The compound according to any one of claims 1-6, wherein R₃ is C₁-C₄ alkyl.

11. The compound according to any one of claims 1-6, wherein R₃ is a methyl.

12. The compound according to any one of claims 1-11, wherein ring A is pyrrolidinyl. azetidinyl, or piperidinyl.

13. The compound according to any one of claims 1-11, wherein ring A is or structure:

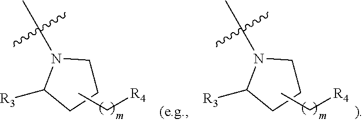

14. The compound according to any one of claims 1-8, wherein ring A is an S-enantiomer of structure:

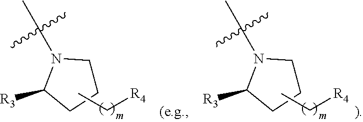

15. The compound according to any one of claims 1-11, wherein ring A is a 2S, 4R-enatiomer of the structure:

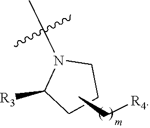

16. The compound according to any one of claim 1-15, wherein m is an integer of 1, 2, or 3.

17. The compound according to any one of claim 1-15, wherein m is an integer of 1 or 2.

18. The compound according to any one of claims 1-15, wherein m is 1.

19. The compound according to any one of claims 1-16, wherein R₃ is —CO₂R₅, —COR₅, —C(O)NR₅R₆, —CONH—OH, —SO₂R₅, —SO₂OR₅, or —SO₂₅R₆,

20. The compound according to any one of claims 1-16, wherein R₃ is —CO₂R₅, —COR₅, —SO₂R₅, —SO₂OR₆, or —SO₂NR₅R₆.

21. The compound according to any one of claims 1-16, wherein R₃ is —CO₂R₅, —SO₂R₅, —SO₂OR₅, or —SO₂NR.R5.

22. The compound according to any one of claims 1-16, wherein R₃ is —CO₂R₅, —COR₅, —C(O)NR₅R₆, or —CONH—OH.

23. The compound according to any one of claims 1-16, wherein R₃ is —CO₂R₅, —C(O)NR₅R₆, or —CONH—OH.

24. The compound according to any one of claims 1-16, wherein R₃ is —CO₂R₅ or —C(O)NR₅R₆.

25. The compound according to any one of claims 1-16, wherein R₃ is —CO₂R₅.

26. The compound according to any one of claims 1-25, wherein each R₅ is independently hydrogen or methyl, and each Ri is independently hydrogen or methyl.

27. The compound according to any one of claims 1-16, wherein R₃ is —CO₂H.

28. The compound according to any one of claims 1-27, wherein R₃ is selected from —C(O)NR₆R₇, —CO₂R₇, and —SO₂NR₆R₇.

29. The compound according to any one of claims 1-27, wherein R₄ is —C(O)NNR₆R₇ or —SONR₆R₇.

30. The compound according to any one of claim 1-27, wherein R₃ is —C(O)NR₆R₇.

31. The compound according to any one of claims 1-30, wherein Ri is hydrogen or C₁-C₄ alkyl.

32. The compound according to any one of claims 1 to 30, wherein R₆ is hydrogen.

33. The compound according to any one of claims 1 to 30, wherein R₆ is methyl.

34. The compound according to any one of claims 1 to 33, wherein R₇ is selected from the group consisting of aryl(C₀-C₁ alkyl) optionally substituted with one or more Rg. heteroaryl(C₀-C₁ alkyl) optionally substituted with one or more R₉, heterocyclyl(C₀-C₁ alkyl) optionally substituted with one or more R₆, and cycloalkyl(C₀-C₁ alkyl) optionally substituted with one or more R₈.

35. The compound according to any one of claims 1 to 33, wherein R₇ is selected from the group consisting of aryl substituted with one or more R₉, aryl-methyl- optionally substituted with one or more R₅, monocyclic heteroaryl(C₀-C₁ alkyl) substituted with one or more R₅, bicyclic heteroaryl(C₀-C₁ alkyl) optionally substituted with one or more R₉, heterocyclyl(C₀-C₁ alkyl) optionally substituted with one or more R₈, and cycloalkyl(C₀-C₁ alkyl) substituted with one or more R₈.

36. The compound according to any one of claims 1 to 33, wherein R₇ is selected from the group consisting of aryl optionally substituted with one or more R₉, heteroaryl optionally substituted with one or more R₈, heterocyclyl optionally substituted with one or more R₆, and cycloalkyl optionally substituted with one or more R₈.

37. The compound according to any one of claims 1 to 33, wherein R₇ is selected from the group consisting of phenyl optionally substituted with one or more R₉, 5 to 12 membered heteroaryl optionally substituted with one or more R₉, 5 to 12 membered heterocyclyl optionally substituted with one or more R₈, and C₃-C₈ cycloalkyl optionally substituted with one or more R₈.

38. The compound according to any one of claims 1 to 33, wherein R₇ is selected from the group consisting of phenyl optionally substituted with one or more R₈, 5 to 12 membered heteroaryl optionally substituted with one or more R₉, and C₃-C₈ cycloalkyl optionally substituted with one or more Ra.

39. The compound according to any one of claims 1 to 33, wherein R₂ is C₃-C₈ cycloalkyl optionally substituted with one or more Re.

40. The compound according to any one of claims 1 to 33, wherein R₂ is phenyl optionally substituted with one or more R₉ or a 5 to 12 membered heteroaryl optionally substituted with one or more R₉.

41. The compound according to any one of claims 1 to 33, wherein R? is phenyl optionally substituted with one or more R₉; or wherein R₂ is phenyl substituted with one or more R₉.

42. The compound according to any one of claims 1 to 33, wherein R₇ is 5 to 12 membered heteroaryl optionally substituted with one or more R₉.

43. The compound according to any one of claims 1 to 33, wherein R is bicyclic heteroaryl optionally substituted with one or more Re.

44. The compound according to any one of claims 1 to 33, wherein R₇ is phenyl substituted with one or more R₉ or bicyclic heteroaryl optionally substituted with one or more R₉.

45. The compound according to any one of claims 1 to 33, wherein R₂ is phenyl substituted with one or more R₉,or indolyl, indazolyl, benzothiazolyl, benzimidazolyl, quinolinyl, or isoquinolinyl, each optionally substituted with one or more R₉.

46. The compound according to any one of claims 1 to 33, wherein R₂ is phenyl substituted with one or more R₉, pyridinyl substituted with one or more R₈, or indolyl, indazolyl, benzothiazolyl, benzimidazolyl, quinolinyl, or isoquinolinyl, each optionally substituted with one or more Rs.

47. The compound according to any one of claims 1 to 46, wherein each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —C(O)R₆, —C(O)OR₅, and —C(O)NR₅R₆, or two R₆ form an oxo.

48. The compound according to any one of claims 1 to 46, wherein each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and ₁-C₆ haloalkoxy, or two R₃ form an oxo.

49. The compound according to any one of claims 1 to 46, wherein each R₆ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, and C₁-C₄ haloalkoxy.

50. The compound according to any one of claims 1 to 49, wherein each R₃ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy C₁-C₆ haloalkoxy, aryl, aryl-methyl-, heteroaryl, heteroaryl-methyl-, and heterocyclyl each optionally substituted with one or more Ric.

51. The compound according to any one of claims 1 to 50, wherein each R₉ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyi, piperazinyl, pyrazolyl, pyrimidinyl, indazolyl, pyridazinyl, imidazolyl, and 2-oxooxazolidinyl, each optionally substituted with one or more R₁₀.

52. The compound according to any one of claims 1 to 51, wherein
m is an integer of 1;
n is an integer of 0 or 1;
ring A represents a pyrrolidinyl, azetidinyl, or piperidinyl ring;
each R, is independently selected C₁-C₃ alkyl, —OH, and C₁-C₃ alkoxy;
R₂ is hydrogen, C₁-C₄ alkyl, or C₁-C₄ haloalkyl;
R₃ is —CO₂R₃ or —C(O)NR₅R₆; and
R₄ is —C(O)NNR₆R₇ or —SO₂NR₆R₇,
wherein
R₅ is hydrogen or C₁-C₄ alkyl;
R₆ is hydrogen, C₁-C₄ alkyl, or C₁-C₄ haloalkyl;
R₇ is selected from the group consisting of phenyl optionally substituted with one or more R₉, 5 to 12 membered heteroaryl optionally substituted with one or more R₉, 5 to 12 membered heterocyclyl optionally substituted with one or more R₆, and C₃-C₈ cycloalkyl optionally substituted with one or more Rs;
each R₆ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy, or two R₃ form an oxo; and
each R₆ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazoly, pyridazinyl, imidazolyl, and 2-oxooxazolidinyl, each optionally substituted with one or more R₁₀.

53. The compound according to any one of claims 1 to 51, wherein
m is an integer of 1;
n is an integer of 0 or 1;
ring A represents a pyrrolidinyl, azetidinyl, or piperidinyl ring;
each R₁ is independently selected from C₁-C₃ alkyl, —OH, and C₁-C₃ alkoxy;
R₂ is hydrogen, C₁-C₄ alkyl, or C₁-C₄ haloalkyl;
R₃ is —CO₂R₅ or —C(O)NR₅R₆; and
R₄ is —C(O)NNR₆R₇ or —SO₂NR₆R₇.
wherein
R₅ is hydrogen or C₁-C₄ alkyl;
R₆ is hydrogen, C₁-C₄ alkyl, or C₁-C₄ haloalkyl;
R₇ is selected from the group consisting of phenyl optionally substituted with one or more R₉, pyridinyl, indolyl, indazolyl, benzothiazolyl, benzimidazolyl, quinolinyl, or isoquinolinyl, each optionally substituted with one or more Rg. and cyclopentyl optionally substituted with one or more Ra:
each R₃ is independently selected from the group consisting of halogen, —NO₂, —CN. C₁-C₆ alkyl, C₁-C₄ haloalkyl, —NH₂, —NH(C₁-C₆ alkyl), —N(C₁-C₆ alkyl)₂, —OH, C,-Cs alkoxy, and C₁-C₆ haloalkoxy, or two R₃ form an oxo; and
each R₃ is independently selected from the group consisting of halogen, C₁-C₄ alkyl, C₁-C₄ haloalkyl, —NH₂, —NH(C₁-C₄ alkyl), —N(C₁-C₄ alkyl)₂, —OH, C₁-C₄ alkoxy, C₁-C₄ haloalkoxy, phenyl, pyridinyl, phenylmethyl, pyridinylmethyl, piperidinyl, morpholinyl, piperazinyl, pyrazolyl, pyrimidinyl, indazolyl, pyridazinyl, imidazolyl, and 2-oxooxazolidinyl, each optionally substituted with one or more R₁₀.

54. The compound of claim 1, which is:
(2S,4R)-4-(2-((1H-indol-5-yl)amino)-2-oxyethyl)-1-(2-methylbenzofuro[3,2-d]pyrimidin-4-yl)pyrrolidine-2-carboxylic acid;
(2S,4R)-4-(2-(cyclopentyl(methyl)amino)-2-oxoethyl)-1-(2-methylbenzofuro[3,2-d]pyrimidin-4-yl)pyrrolidine-2-carboxylic acid;
(2S,4R)-4-(2-((1H-indazol-5-yl)amino)-2-oxyethyl)-1-(2-methylbenzofuro[3,2-d]pyrimidin-4-yl)pyrrolidine-2-carboxylic acid;
(2S,4R)-4-(2-(benzo[d]thiazol-6-ylamino)-2-oxyethyl)-1-(2-methylbenzofuro[3,2-d]pyrimidin-4-yl)pyrrolidine-2-carboxylic acid;
(2S,4R)-4-(2-([1,1′-biphenyl]-4-ylamino)-2-oxyethyl)-1-(2-methylbenzofuro[3,2-d]pyrimidin-4-yl)pyrrolidine-2-carboxylic acid;
(2S,4R)-4-(2-((2-methoxypyridin-4-yl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(piperidin-1-yl)phenyl)amino)ethyl)pyrrolidine-2-carboxylic acid;
(2S,4R)-1-(2-methylbenzofuro[3,2-d]pyrimidin-4-yl)-4-(2-oxo-2-((4-(piperazin-1-yl)phenyl)amino)ethyl)pyrrolidine-2-carboxylic acid;
(2S,4R)-1-(2-methylbenzofuro[3,2-d]pyrimidin-4-yl)-4-(2-((4-(4-methylpiperazin-1-yl)phenyl)amino)-2-oxoethyl)pyrrolidine-2-carboxylic acid;
(2S,4R)-4-(2-((4-(1H-pyrazol-4-yl)phenyl)amino)-2-oxoethyl)-1-(2-methylbenzofuro[3,2-d]pyrimidin-4-yl)pyrrolidine-2-carboxylic acid;
(2S,4R)-4-(2-((3-(1H-pyrazol-4-yl)phenyl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(trifluoromethoxy)phenyl)amino)ethyl)pyrrolidine-2-carboxylic acid;
(2S,4R)-4-(2-((3-methoxyphenyl)amino)-2-oxoethyl)-1-(2-methylbenzofuro[3,2-d]pyrimidin-4-yl)pyrrolidine-2-carboxylic acid;
(2S,4R)-4-(2-((2-methoxyphenyl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-(p-tolylamino)ethyl)pyrrolidine-2-carboxylic acid;
(2S,4R)-1-(2-methylbenzofuro[3,2-d]pyrimidin-4-yl)-4-(2-oxo-2-(m-tolylamino)ethyl)pyrrolidine-2-carboxylic acid;
(2S,4R)-1-(2-methylbenzofuro[3,2-d]pyrimidin-4-yl)-4-(2-oxo-2-(o-tolylamino)ethyl)pyrrolidine-2-carboxylic acid;
(2S,4R)-4-(2-((4-fluorophenyl)amino)-2-oxoethyl)-1-(2-methylbenzofuro[3,2-d]pyrimidin-4-yl)pyrrolidine-2-carboxylic acid;
(2S,4R)-4-(2-((3-fluorophenyl)amino)-2-oxoethyl)-1-(2-methylbenzofuro[3,2-d]pyrimidin-4-yl)pyrrolidine-2-carboxylic acid;
(2S,4R)-4-(2-((2-fluorophenyl)amino)-2-oxoethyl)-1-(2-methylbenzofuro[3,2-d]pyrimidin-4-yl)pyrrolidine-2-carboxylic acid;
(2S,4R)-4-(2-((4-chlorophenyl)amino)-2-oxoethyl)-1-(2-methylbenzofuro[3,2-d]pyrimidin-4-yl)pyrrolidine-2-carboxylic acid;
(2S,4R)-4-(2-((3-chlorophenyl)amino)-2-oxoethyl)-1-(2-methylbenzofuro[3,2-d]pyrimidin-4-yl)pyrrolidine-2-carboxylic acid;
(2S,4R)-4-(2-((2-chlorophenyl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(trifluoromethyl)phenyl)amino)ethyl)pyrrolidine-2-carboxylic acid;
(2S,4R)-1-(2-methylbenzofuro[3,2-d]pyrimidin-4-yl)-4-(2-oxo-2-((3-(trifluoromethyl)phenyl)amino)ethyl)pyrrolidine-2-carboxylic acid;
(2S,4R)-4-(2-((3,4-dichlorophenyl)amino)-2-oxoethyl)-1-(2-methylbenzofuro[3,2-d]pyrimidin-4-yl)pyrrolidine-2-carboxylic acid;
(2S,4R)-4-(2-((4-ethynylphenyl)amino)-2-oxoethyl)-1-(2-methylbenzofuro[3,2-d]pyrimidin-4-yl)pyrrolidine-2-carboxylic acid;
(2S,4R)-4-(2-((1H-indazol-4-yl)amino)-2-oxoethyl)-1-(2-methylbenzofuro[3,2-d]pyrimidin-4-yl)pyrrolidine-2-carboxylic acid;
(2S,4R)-4-(2-((1H-benzo[d]imidazol-6-yl)amino)-2-oxoethyl)-1-(2-methylbenzofuro[3,2-d]pyrimidin-4-yl)pyrrolidine-2-carboxylic acid;
(2S,4R)-4-(2-((1H-indazol-6-yl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-(naphthalen-2-ylamino)-2-oxoethyl)pyrrolidine-2-carboxylic acid;
(2S,4R)-1-(2-methylbenzofuro[3,2-d]pyrimidin-4-yl)-4-(2-oxo-2-(quinolin-6-ylamino)ethyl)pyrrolidine-2-carboxylic acid;
(2S,4R)-4-(2-(isoquinolin-6-ylamino)-2-oxoethyl)-1-(2-methylbenzofuro[3,2-d]pyrimidin-4-yl)pyrrolidine-2-carboxylic acid;
(2S,4R)-4-(2-(isoquinolin-7-ylamino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-(quinolin-7-ylamino)ethyl)pyrrolidine-2-carboxylic acid;
(2S,4R)-4-(2-([1,1′-biphenyl]-3-ylamino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(pyridin-3-yl)phenyl)amino)ethyl)pyrrolidine-2-carboxylic acid;
(2S,4R)-4-(2-((4-(1H-indazol-5-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(1H-indazol-6-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(6-aminopyridin-3-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(2-aminopyridin-4-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(6-aminopyridin-2-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(2-aminopyridin-3-yl)phenyl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(pyridazin-3-yl)phenyl)amino)ethyl)pyrrolidine-2-carboxylic acid;
(2S,4R)-1-(2-methylbenzofuro[3,2-d]pyrimidin-4-yl)-4-(2-oxo-2-((4-(pyridazin-4-yl)phenyl)amino)ethyl)pyrrolidine-2-carboxylic acid;
(2S,4R)-4-(2-((4-(1H-indazol-4-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(6-hydroxypyridin-3-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-(2-hydroxypyridin-4-yl)phenyl)amino)-2-oxoethyl)-1-(2-methylbenzofuro[3,2-d]pyrimidin-4-yl)pyrrolidine-2-carboxylic acid;
(2S,4R)-4-(2-((3-(1H-imidazol-4-yl)phenyl)amino)-2-oxoethyl)-1-(2-methylbenzofuro[3,2-d]pyrimidin-4-yl)pyrrolidine-2-carboxylic acid;
(2S,4R)-4-(2-((3′-methoxy-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-4-(2-((3′-hydroxy-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(2-oxooxazolidin-3-yl)phenyl)amino)ethyl)pyrrolidine-2-carboxylic acid;
(2S,4R)-4-(2-((4-(4-hydroxypiperidin-1-yl)phenyl)amino)-2-oxoethyl)-1-(2-methylbenzofuro[3,2-d]pyrimidin-4-yl)pyrrolidine-2-carboxylic acid;
(2S,4R)-4-(2-((4-benzylphenyl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((4-(pyridin-3-ylmethyl)phenyl)amino)ethyl)pyrrolidine-2-carboxylic acid;
(2S,4R)-1-(2-methylbenzofuro[3,2-d]pyrimidin-4-yl)-4-(2-oxo-2-((4-(pyridin-2-ylmethyl)phenyl)amino)ethyl)pyrrolidine-2-carboxylic acid;
(2S,4R)-1-(2-methylbenzofuro[3,2-d]pyrimidin-4-yl)-4-(2-((3-morpholinophenyl)amino)-2-oxoethyl)pyrrolidine-2-carboxylic acid;
(2S,4R)-4-(2-((3-(4-hydroxypiperidin-1-yl)phenyl)amino)-2-oxoethyl)-1-(2-methylbenzofuro[3,2-d]pyrimidin-4-yl)pyrrolidine-2-carboxylic acid;
(2S,4R)-4-(2-((3′-(hydroxymethyl)-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-4-(2-((4′-(hydroxymethyl)-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-4-(2-((2-methoxy-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-4-(2-((2-amino-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((2-(trifluoromethyl)-[1. 1′-biphenyl]-4-yl)amino)ethyl)pyrrolidine-2-carboxylic acid;
(2S,4R)-1-(2-methylbenzofuro[3,2-d]pyrimidin-4-yl)-4-(2-oxo-2-((6-phenylpyridazin-3-yl)amino)ethyl)pyrrolidine-2-carboxylic acid;
(2S,4R)-1-(2-methylbenzofuro[3,2-d]pyrimidin-4-yl)-4-(2-oxo-2-((2-phenylpyrimidin-5-yl)amino)ethyl)pyrrolidine-2-carboxylic acid;
(2S,4R)-4-(2-((2-fluoro-[1,1′-biphenyl]-4-yl)amino)-2-oxoethyl)-1-(2-methylbenzofuro[3,2-d]pyrimidin-4-yl)pyrrolidine-2-carboxylic acid;
(2S,4R)-1-(2-methylbenzofuro[3,2-d]pyrimidin-4-yl)-4-(2-oxo-2-((5-phenylpyridin-2-yl)amino)ethyl)pyrrolidine-2-carboxylic acid;
or a pharmaceutically acceptable salt, N-oxide, and/or a solvate or hydrate thereof.

55. The compound of any of claims 1-54, wherein the compound is in the form of an N-oxide.

56. The compound of any of claims 1-55, wherein the compound is in the form of a pharmaceutically acceptable salt.

57. The compound of any of claims 1-56, wherein the compound is in the form of the base compound.

58. The compound of any of claims 1-56, wherein the compound is in the form of solvate or hydrate.

59. The compound of any of claims 1-58, wherein the compound has an improved inhibition of cGAS activation in presence of Mn²⁺ compared to activation in absence of Mn²⁺ (optionally having an IC₅₀ in the presence of Mn²⁺ that is at least 5-fold more than the ICss of the compound in otherwise identical conditions but lacking Mn²⁺).

60. A pharmaceutical composition comprising a compound according to any one of claims 1-59 and a pharmaceutically acceptable carrier, solvent, adjuvant or diluent.

61. A method for treating or preventing inappropriate activation of a type I interferon (IFN) response in a subject in need thereof, the method comprising administering to a subject in need of such treatment an effective amount of one or more compounds according to any one of claims 1-59 or a pharmaceutical composition according to claim 60.

62. A method of treating an autoimmune disorder, the method comprising administering to a subject in need of such treatment an effective amount of one or more compounds according to any one of claims 1-59 or a pharmaceutical composition according to claim 60.

63. The method of claim 62, wherein the autoimmune disorder is Aicardi-Goutieres Syndrome, retinal vasculopathy with cerebral leukodystropy, lupus erythematosus, scleroderma, Sjögren's syndrome, age-related macular degeneration, pancreatitis, ischemia, inflammatory bowel disease, nonalcoholic steatohepatitis, or Parkinson's disease.
